(19) United States
(12) Patent Application Publication
(10) Pub. No.: US 2026/0345596 A1
(43) Pub. Date: Nov. 5, 2026

(54) TREATMENT OF LOCALLY ADVANCED OR METASTATIC UROTHELIAL CARCINOMA WITH ERDAFINITIB

(71) Applicant: Janssen Pharmaceutica NV, Beerse (BE)

(72) Inventors: Spyridon TRIANTOS, Spring House, PA (US); Kris Emanuel D DEPRINCE, Beerse (BE)

(21) Appl. No.: 19/486,441

(22) PCT Filed: May 24, 2024
(86) PCT No.: PCT/IB2024/055103

§ 371 Date: Nov. 21, 2025

Related U.S. Application Data
(60) Provisional application No. 63/504,050, filed on May 24, 2023.
(60) Provisional application No. 63/590,334, filed on Oct. 13, 2023.

(30) Foreign Application Priority Data
Oct. 23, 2023 (CA) 3217575

(51) Int. Cl. A61K 31/498; A61K 9/00; A61K 9/28; A61P 35/04

(52) U.S. Cl. CPC A61K 31/498; A61K 9/0053; A61K 9/28; A61P 35/04

(57) ABSTRACT

The present invention relates to the treatment of locally advanced or metastatic urothelial carcinoma (mUC) with erdafitinib in patients with FGFR altered mUC who progressed on or after 1 or more prior treatments that included a checkpoint inhibitor, in particular an anti-PD-(L)1 agent.

### TECHNICAL FIELD

The present invention relates to, among other things, the treatment of locally advanced or metastatic urothelial carcinoma (mUC) with erdafitinib in patients with FGFR altered mUC who progressed on or after 1 or more prior treatments that included a checkpoint inhibitor, in particular an anti-PD-(L)1 agent.

### BACKGROUND

For patients with metastatic urothelial carcinoma, or mUC, first-line systemic therapy includes platinum-based chemotherapy with or without maintenance avelumab for platinum-eligible population. Platinum-ineligible patients may receive anti-PD-1 or anti-PD-L1 therapy, where approved, or the combination of pembrolizumab and enfortumab vedotin, where approved. Second-line systemic therapy includes checkpoint inhibitors for the checkpoint inhibitor-naïve population. For patients with prior checkpoint inhibitor treatment, second-line systemic therapy includes single-agent chemotherapy (taxanes, vinflunine), or where approved, enfortumab vedotin, sacituzumab govitecan, and erdafitinib.

Hence, checkpoint inhibitors are used in both the first- and second-line settings for the treatment of mUC, but only approximately 30% of patients with mUC respond to immune checkpoint inhibitor treatment. Treatment options after progression on checkpoint inhibitors are limited. In the real-world setting, only ~30% of patients received subsequent anticancer treatment after checkpoint inhibitor discontinuation. There is clear unmet need to extend treatment options for patients post anti-PD-1 or anti-PD-L1 therapy.

WO2011/135376 describes quinoxaline derivative compounds for the treatment of cancer. WO2016/161239 describes FGFR/PD-1 combination therapy for the treatment of cancer. WO2018/141921 describes the treatment of cancer with erdafitinib. WO2021/058798 describes the use of FGFR inhibitors in FGFR-genetically altered cancers to enhance patient response to immune check point inhibitors in sequential treatment settings.

### DISCLOSURE OF THE INVENTION

This application has been drafted in sections to aid readability. However, this does not mean that each section is to be read in isolation. To the contrary, unless otherwise specified, each section is to be read with cross-referencing to the other sections, i.e. taking the entire application as a whole. No artificial separation of embodiments is intended, unless explicitly stated.

Thus, all of the embodiments described herein relating to an aspect of the invention apply equally to, i.e. are also disclosed in relation to/combination with, the other aspects described herein.

### Certain Terminology

The transitional terms “comprising”, “consisting essentially of”, and “consisting” are intended to connote their generally accepted meanings in the patent vernacular; that is, (i) “comprising”, which is synonymous with “including”, “containing”, or “characterized by”, is inclusive or open-ended and does not exclude additional, unrecited elements or method steps; (ii) “consisting of” excludes any element, step, or ingredient not specified in the claim or embodiment; and (iii) “consisting essentially of” limits the scope of a claim or embodiment to the specified materials or steps “and those that do not materially affect the basic and novel characteristic(s)” of the claimed invention or embodiment. More specifically, the basic and novel characteristics relates to the ability of the method to provide at least one of the benefits described herein, including but not limited to the ability to improve the survivability of the human population relative to the survivability of the comparative human population described elsewhere herein. Embodiments described in terms of the phrase “comprising” (or its equivalents), also provide, as embodiments, those which are independently described in terms of “consisting of” and “consisting essentially of”.

When a value is expressed as an approximation by use of the descriptor “about”, it will be understood that the particular value forms another embodiment. If not otherwise specified, the term “about” signifies a variance of 10% of the associated value, but additional embodiments include those where the variance may be ±5%, ±15%, ±20%, ±25%, or ±50%, in particular the term “about” signifies a variance of ±5% or ±10% of the associated value, more in particular ±5%.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used herein, the singular forms “a,” “an,” and “the” include the plural.

The following abbreviations are used throughout the disclosure: FGFR (fibroblast growth factor receptor); FGFR3-TACC3 V1 (fusion between genes encoding FGFR3 and transforming acidic coiled-coil containing protein 3 variant 1); FGFR3-TACC3 V3 (fusion between genes encoding FGFR3 and transforming acidic coiled-coil containing protein 3 variant 3); FGFR3-BAIAP2L1 (fusion between genes encoding FGFR3 and brain-specific angiogenesis inhibitor 1-associated protein 2-like protein 1); FGFR2-BICC1 (fusion between genes encoding FGFR2 and bicaudal C homolog 1); FGFR2-CASP7 (fusion between genes encoding FGFR2 and caspase 7).

As used herein, “patient” is intended to mean any animal, in particular, mammals. Thus, the methods and uses are applicable to human and nonhuman animals, although most preferably with humans. The terms “patient” and “subject” and “human” and “subject in need thereof” may be used interchangeably. By “an adult patient” it is meant a patient aged 18 years and older. In a preferred embodiment, the subject in need or the patient is a human subject in need or a human patient. It is understood by a person skilled in the art that a patient can also mean a population of patients. It is understood by a person skilled in the art that when a median endpoint is mentioned, for example median overall survival or median progression-free survival, that a patient means a population of patients.

The terms “treat” and “treatment” refer to the treatment of a patient afflicted with a pathological condition and refers to an effect that alleviates the condition by killing the cancerous cells, but also to an effect that results in the inhibition of the progress of the condition, and includes a reduction in the rate of progress, a halt in the rate of progress, amelioration of the condition, and cure of the condition. Treatment as a prophylactic measure (i.e., prophylaxis) is also included. In an embodiment, treatment does not include a prophylactic measure.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient.

The term “up-titrate” the dose means to increase the dose.

The term “dosage” refers to the information of the amount of the therapeutic to be taken by the subject and the frequency of the number of times the therapeutic is to be taken by the subject.

The term “dose” refers to the amount or quantity of the therapeutic to be taken each time.

The term “cancer” as used herein refers to an abnormal growth of cells which tend to proliferate in an uncontrolled way and, in some cases, to metastasize (spread).

The term “continuous daily dosing schedule” refers to the administration of a particular therapeutic agent without any drug holidays from the particular therapeutic agent. In some embodiments, a continuous daily dosing schedule of a particular therapeutic agent comprises administration of a particular therapeutic agent every day at roughly the same time each day.

The term “objective response rate” (ORR), is defined as the proportion of subjects who achieve complete response or partial response, as assessed per RECIST v1.1 by the investigator.

The term “confirmed objective response rate by investigator assessment” is defined as greater than or equal to 2 consecutive investigator assessments of ORR.

The term “disease control rate” (DCR) is defined as the percentage of participants who have achieved SD and confirmed and unconfirmed CR and PR.

The term “adverse event” is any untoward medical event that occurs in a participant administered an investigational product, and it does not necessarily indicate only events with clear causal relationship with the relevant investigational product.

The term “complete response” (CR) is defined in the Response Evaluation Criteria in Solid Tumors (RECIST) Version 1.1, i.e., disappearance of all target lesions, with no remnant present and no viable tumor seen on histopathological examination.

The term “partial response” (PR) is defined in the RECIST Version 1.1, i.e. at least a 30% decrease in the sum of diameters of target lesions, taking as reference the baseline sum diameters.

The term “stable disease” (SD) is defined in the RECIST Version 1.1, i.e. neither sufficient shrinkage to qualify for PR nor sufficient increase to qualify for PD, taking as reference the smallest sum diameters while on study.

The term “progressive disease” (PD) is defined in the RECIST Version 1.1, i.e. at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progression.

The term “time to response” (TTR) is defined as the time from the date of randomization to the date of initial documentation of a response (CR or PR).

The term “overall survival” (OS) is defined as the time from the date of randomization to the date of the subject's death. If the subject is alive or the vital status is unknown, the subject will be censored at the date the subject was last known to be alive.

The term “progression-free survival” (PFS) is defined as the duration in days from the date of randomization to the date of disease progression (assessed per RECIST v1.1 by the investigator) or relapse from CR or death, whichever is reported first. For subjects who do not have disease progression and are alive, as well as for subjects with unknown disease progression or unknown survival status as of the clinical cutoff date, PFS will be censored at the date of the last adequate disease assessment. If there is no postbaseline tumor assessment for a subject, PFS will be censored on the date of randomization. Adequate disease assessment is defined as having sufficient evidence to indicate correctly that progression has or has not occurred.

The term “duration of response” (DOR) is defined, for responders, as the duration in days from the date of initial documentation of a response to the date of first documented evidence of progressive disease (or relapse for subjects who experience CR during the study) or death. The censoring is similar to PFS.

The term “safety” refers to the collection of adverse event, clinical laboratory values, electrocardiograms, vital signs, ophthalmologic evaluations, physical examinations.

Change from baseline in patient-reported health status and physical functioning scales of the Functional Assessment of Cancer Therapy-Bladder Cancer (FACT-Bl), Time Until Symptom Deterioration (subset of FACT-Bl items), Patient-Global Impression of Severity (PGIS), and utility and visual analog scale of the European Quality of Life-5 Dimensions-5 Levels Questionnaire (EQ-5D-5L).

The term “randomization” as it refers to a clinical trial refers to the time when the patient is confirmed eligible for the clinical trial and gets assigned to a treatment arm.

The term “NCI CTCAE” refers to National Cancer Institute Common Terminology Criteria for Adverse Events

The terms “kit” and “article of manufacture” are used as synonyms.

The term “biological sample” refers to any sample from a patient in which cancerous cells can be obtained and detection of a FGFR genetic alteration is possible.

Suitable biological samples include, but are not limited to, blood, lymph fluid, bone marrow, a solid tumor sample, or any combination thereof. In some embodiments, the biological sample can be formalin-fixed paraffin-embedded tissue (FFPET).

The term “anti-programmed death-(ligand)1 agent (anti-PD-(L)1) agent) refers to an anti-PD-1 antibody that blocks the interaction between PD-1 and PD-L1 e.g., by specifically binding to PD-1, and suppresses at least one biological activity mediated by PD-1 ligand PD-L1 or PD-L2. The anti-PD-1 antibody is an antagonist when the at least one biological activity mediated by PD-L1 or PD-L2 is suppressed by at least about 20%, 30%, 40%, 45%, 50%, 55%, 60%, 65%, 70%, 75%, 80%, 85%, 90%, 95%, or 100% greater than in the absence of the antagonist (e.g., negative control), or when the suppression is statistically significant when compared to the suppression in the absence of the antagonist. A typical biological activity that is mediated by PD-L1 or PD-L2 binding to PD-1 is inhibition of antigen-specific CD4+ and/or CD8+ T cells. Therefore, antagonistic antibody relieves PD-L1 mediated suppression resulting in enhancement of immune responses.

As used here, “block(s) the interaction” refers to the ability of an anti-PD-1 antibody to inhibit or reduce binding of PD-L1 to PD-1, such that signaling/functioning through PD-1 is abolished or diminished.

The term “specifically binds”, “specific binding” or “binds” refer to antibody binding to an antigen (e.g. PD-1) or an epitope within the antigen with greater affinity than for other antigens. Typically, the antibody binds to the antigen or the epitope within the antigen with an equilibrium dissociation constant (KD) of about 1×10⁻⁸ M or less, for example about 1×10⁻⁹ M or less, about 1×10⁻¹⁰ M or less, about 1×10⁻¹¹ M or less, or about 1×10⁻¹² M or less, typically with a KD that is at least one hundred-fold less than its KD for binding to a non-specific antigen (e.g., BSA, casein). The KD may be measured using standard procedures. Antibodies that specifically bind to the antigen or the epitope within the antigen may, however, have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset).

The term “PD-1” refers to human programmed cell death protein 1, PD-1. PD-1 is also known as CD279 or PDCD1. The amino acid sequence of the mature human PD-1 (without signal sequence) is shown in SEQ ID NO: 1. The extracellular domain spans residues 1-150, the transmembrane domain spans residues 151-171 and the cytoplasmic domain spans residues 172-268 of SEQ ID NO: 1 (Table 1A).

As used herein, “Intent-to-Treat” or “ITT” population refers to all randomized subjects in a given cohort.

TABLE 1A

| | |
|---|---|
| Mature human | PGWFLDSPDRPWNPPTFSPALLVVTEGD |
| PD-1 (without | NATFTCSFSNTSESFVLNWYRMSPSNQT |
| signal sequence) | DKLAAFPEDRSQPGQDCRFRVTQLPNGR |
| (SEQ ID NO: 1) | DFHMSVVRARRNDSGTYLCGAISLAPKA |
| | QIKESLRAELRVTERRAEVPTAHPSPSP |
| | RPAGQFQTLVVGVVGGLLGSLVLLVWVL |
| | AVICSRAARGTIGARRTGQPLKEDPSAV |
| | PVFSVDYGELDFQWREKTPEPPVPCVPE |
| | QTEYATIVFPSGMGTSSPARRGSADGPR |
| | SAQPLRPEDGHCSWPL |

“Antibodies” is meant in a broad sense and includes immunoglobulin molecules belonging to any class, IgA, IgD, IgE, IgG and IgM, or sub-class IgA1, IgA2, IgG1, IgG2, IgG3 and IgG4 and including either kappa (κ) and lambda (λ) light chain. Antibodies include monoclonal antibodies, full length antibodies, antigen binding fragments, bispecific or multispecific antibodies, dimeric, tetrameric or multimeric antibodies, single chain antibodies, domain antibodies and any other modified configuration of the immunoglobulin molecule that comprises an antigen binding fragment of the required specificity. “Full length antibodies” are comprised of two heavy chains (HC) and two light chains (LC) inter-connected by disulfide bonds. Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL may be further subdivided into regions of hypervariability, termed complementarity determining regions (CDR), interspersed with framework regions (FR). Each VH and VL is composed of three CDRs and four FR segments, arranged from amino-terminus to carboxy-terminus in the following order: FR1, CDR1, FR2, CDR2, FR3, CDR3, and FR4. Antibodies include antibodies generated using various technologies, including antibodies generated from immunized mice or rat or identified from phage or mammalian display libraries as described herein.

“Complementarity determining regions (CDR)” are antibody regions that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al. (1970) J Exp Med 132: 211-50) (Kabat et al., Sequences of Proteins of Immunological Interest, 5th Ed. Public Health Service, National Institutes of Health, Bethesda, Md., 1991), Chothia (Chothia et al. (1987) J Mol Biol 196: 901-17), IMGT (Lefranc et al. (2003) Dev Comp Immunol 27: 55-77) and AbM (Martin and Thornton (1996) J Bmol Biol 263: 800-15). The correspondence between the various delineations and variable region numbering are described (see e.g. Lefranc et al. (2003) Dev Comp Immunol 27: 55-77; Honegger and Pluckthun, (2001) J Mol Biol 309:657-70; International ImMunoGeneTics (IMGT) database; Web resources, http://www_imgt_org). Available programs such as abYsis by UCL Business PLC may be used to delineate the CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification.

“Antigen binding fragment” refers to a portion of an immunoglobulin molecule that retains the antigen binding properties of the parental full-length antibody. Exemplary antigen binding fragments are heavy chain complementarity determining regions (HCDR) 1, 2 and/or 3, light chain complementarity determining regions (LCDR) 1, 2 and/or 3, the VH, the VL, the VH and the VL, Fab, F(ab′)2, Fd and Fv fragments as well as domain antibodies (dAb) consisting of either one VH domain or one VL domain. The VH and the VL domains may be linked together via a synthetic linker to form various types of single chain antibody designs in which the VH/VL domains pair intramolecularly, or intermolecularly in those cases when the VH and VL domains are expressed by separate chains, to form a monovalent antigen binding site, such as single chain Fv (scFv) or diabody; described for example in Int. Pat. Publ. No. WO1998/44001, Int. Pat. Publ. No. WO1988/01649; Int. Pat. Publ. No. WO1994/13804; Int. Pat. Publ. No. WO1992/01047.

“Humanized antibody” refers to an antibody in which CDR sequences are derived from non-human species and the frameworks are derived from human immunoglobulin sequences. Humanized antibody may include substitutions in the framework so that the framework may not be an exact copy of expressed human immunoglobulin or human immunoglobulin germline gene sequences. Antibodies in which at least one CDR is derived from non-human species and at least one framework is derived from human immunoglobulin sequences are humanized antibodies. Humanized antibody may include substitutions in the frameworks so that the frameworks may not be exact copies of expressed human immunoglobulin or human immunoglobulin germline gene sequences.

“Human antibody” refers to an antibody that is optimized to have minimal immune response when administered to a human subject. Variable regions of human antibody are derived from human germline immunoglobulin sequences. If the antibody contains a constant region or a portion of the constant region, the constant region is also derived from human germline immunoglobulin sequences.

Human antibody comprises heavy or light chain variable regions that are “derived from” human germline immunoglobulin sequences if the variable regions of the antibody are obtained from a system that uses human germline immunoglobulin genes. Such exemplary systems are human immunoglobulin gene libraries displayed on phage or mammalian cells, and transgenic non-human animals such as mice, rats or chicken carrying human immunoglobulin loci. “Human antibody” typically contains amino acid differences when compared to the immunoglobulins expressed in humans due to differences between the systems used to obtain the antibody and human immunoglobulin loci, introduction of naturally occurring somatic mutations, intentional introduction of substitutions into the framework or the CDRs. “Human antibody” is typically about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin sequences. In some cases, “human antibody” may contain consensus framework sequences derived from human framework sequence analyses, for example as described in (Knappik et al. (2000) J Mol Biol 296: 57-86), or synthetic HCDR3 incorporated into human immunoglobulin gene libraries displayed on phage, for example as described in (Shi et al. (2010) J Mol Biol 397: 385-96), and in Int. Patent Publ. No. WO2009/085462. Antibodies in which CDRs are derived from a non-human species are not included in the definition of “human antibody”.

“Monoclonal antibody” refers to an antibody population with single amino acid composition in each antibody chain except for possible well-known alterations such as removal of C-terminal lysine from the antibody heavy chain or alterations due to post-translational modification(s) of amino acids, such as methionine oxidation or asparagine or glutamine deamidation. Monoclonal antibodies typically specifically bind one antigenic epitope, except that bispecific or multispecific monoclonal antibodies specifically bind two or more distinct antigenic epitopes. Monoclonal antibodies may have heterogeneous glycosylation within the antibody population. Monoclonal antibody may be monospecific or multispecific, or monovalent, bivalent or multivalent. A bispecific antibody is included in the term monoclonal antibody.

“Isolated” refers to a homogenous population of molecules (such as synthetic polynucleotides or a protein such as an antibody) which have been substantially separated and/or purified away from other components of the system the molecules are produced in, such as a recombinant cell, as well as a protein that has been subjected to at least one purification or isolation step. “Isolated antibody” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

As used herein, “FGFR genetic alteration” refers to an alteration in the wild type FGFR gene, including FGFR fusion genes, FGFR mutations, or any combination thereof. The terms “variant” and “alteration” are used interchangeably herein.

In certain embodiments, the FGFR2 or FGFR3 genetic alteration is an FGFR gene fusion. “FGFR fusion” or “FGFR gene fusion” refers to a gene encoding a portion of FGFR (e.g., FGRF2 or FGFR3) and one of the herein disclosed fusion partners, or a portion thereof, created by a translocation between the two genes. The terms “fusion” and “translocation” are used interchangeable herein. The presence of one or more of the following FGFR fusion genes in a biological sample from a patient can be determined using the disclosed methods or uses or by methods known to those of ordinary skill in the art: FGFR3-TACC3, FGFR3-BAIAP2L1, FGFR2-BICC1, FGFR2-CASP7, or any combination thereof. In certain embodiments, FGFR3-TACC3 is FGFR3-TACC3 variant 1 (FGFR3-TACC3 V1) or FGFR3-TACC3 variant 3 (FGFR3-TACC3 V3). Table 2A provides the FGFR fusion genes and the FGFR and fusion partner exons that are fused. The nucleotide sequences of the individual FGFR fusion genes are disclosed in Table 3A. The underlined sequences correspond to either FGFR3 or FGFR2, the sequences in black represent the fusion partners.

TABLE 2A

| Fusion Gene | FGFR Exon | Partner Exon |
|---|---|---|
| FGFR2 | | |
| FGFR2-BICC1 | 19 | 3 |
| FGFR2-CASP7 | 19 | 4 |
| FGFR3 | | |
| FGFR3-BAIAP2L1 | 18 | 2 |
| FGFR3-TACC3 V1 | 18 | 11 |
| FGFR3-TACC3 V3 | 18 | 10 |

TABLE 3A

| | |
|---|---|
| FGFR3-TACC3 | >ATGGGCGCCCCTGCCTGCGCCCTCGCGCTCTGCGTGGCCGTGGCC |
| V1 (2850 base | ATCGTGGCCGGCGCCTCCTCGGAGTCCTTGGGGACGGAGCAGCGC |
| pairs) | GTCGTGGGGCGAGCGGCAGAAGTCCCGGGCCCAGAGCCCGGCCAG |
| (SEQ ID NO: 2) | CAGGAGCAGTTGGTCTTCGGCAGCGGGGATGCTGTGGAGCTGAGC |
| | TGTCCCCCGCCCGGGGGTGGTCCCATGGGGCCCACTGTCTGGGTCA |
| | AGGATGGCACAGGGCTGGTGCCCTCGGAGCGTGTCCTGGTGGGGC |
| | CCCAGCGGCTGCAGGTGCTGAATGCCTCCCACGAGGACTCCGGGG |
| | CCTACAGCTGCCGGCAGCGGCTCACGCAGCGCGTACTGTGCCACTT |
| | CAGTGTGCGGGTGACAGACGCTCCATCCTCGGGAGATGACGAAGA |
| | CGGGGAGGACGAGGCTGAGGACACAGGTGTGGACACAGGGGCCC |
| | CTTACTGGACACGGCCCGAGCGGATGGACAAGAAGCTGCTGGCCG |
| | TGCCGGCCGCCAACACCGTCCGCTTCCGCTGCCCAGCCGCTGGCAA |
| | CCCCACTCCCTCCATCTCCTGGCTGAAGAACGGCAGGGAGTTCCGC |
| | GGCGAGCACCGCATTGGAGGCATCAAGCTGCGGCATCAGCAGTGG |
| | AGCCTGGTCATGGAAAGCGTGGTGCCCTCGGACCGCGGCAACTAC |
| | ACCTGCGTCGTGGAGAACAAGTTTGGCAGCATCCGGCAGACGTAC |
| | ACGCTGGACGTGCTGGAGCGCTCCCCGCACCGGCCCATCCTGCAG |
| | GCGGGGCTGCCGGCCAACCAGACGGCGGTGCTGGGCAGCGACGTG |
| | GAGTTCCACTGCAAGGTGTACAGTGACGCACAGCCCCACATCCAG |
| | TGGCTCAAGCACGTGGAGGTGAATGGCAGCAAGGTGGGCCCGGAC |
| | GGCACACCCTACGTTACCGTGCTCAAGACGGCGGGCGCTAACACC |
| | ACCGACAAGGAGCTAGAGGTTCTCTCCTTGCACAACGTCACCTTTG |
| | AGGACGCCGGGGAGTACACCTGCCTGGCGGGCAATTCTATTGGGT |
| | TTTCTCATCACTCTGCGTGGCTGGTGGTGCTGCCAGCCGAGGAGGA |
| | GCTGGTGGAGGCTGACGAGGCGGGCAGTGTGTATGCAGGCATCCT |
| | CAGCTACGGGGTGGGCTTCTTCCTGTTCATCCTGGTGGTGGCGGCT |
| | GTGACGCTCTGCCGCCTGCGCAGCCCCCCCAAGAAAGGCCTGGGC |
| | TCCCCCACCGTGCACAAGATCTCCCGCTTCCCGCTCAAGCGACAGG |
| | TGTCCCTGGAGTCCAACGCGTCCATGAGCTCCAACACACCACTGGT |
| | GCGCATCGCAAGGCTGTCCTCAGGGGAGGGCCCCACGCTGGCCAA |
| | TGTCTCCGAGCTCGAGCTGCCTGCCGACCCCAAATGGGAGCTGTCT |
| | CGGGCCCGGCTGACCCTGGGCAAGCCCCTTGGGGAGGGCTGCTTC |
| | GGCCAGGTGGTCATGGCGGAGGCCATCGGCATTGACAAGGACCGG |
| | GCCGCCAAGCCTGTCACCGTAGCCGTGAAGATGCTGAAAGACGAT |
| | GCCACTGACAAGGACCTGTCGGACCTGGTGTCTGAGATGGAGATG |
| | ATGAAGATGATCGGGAAACACAAAAACATCATCAACCTGCTGGGC |
| | GCCTGCACGCAGGGCGGGCCCCTGTACGTGCTGGTGGAGTACGCG |
| | GCCAAGGGTAACCTGCGGGAGTTTCTGCGGGCGCGGCGGCCCCCG |
| | GGCCTGGACTACTCCTTCGACACCTGCAAGCCGCCCGAGGAGCAG |
| | CTCACCTTCAAGGACCTGGTGTCCTGTGCCTACCAGGTGGCCCGGG |
| | GCATGGAGTACTTGGCCTCCCAGAAGTGCATCCACAGGGACCTGG |
| | CTGCCCGCAATGTGCTGGTGACCGAGGACAACGTGATGAAGATCG |
| | CAGACTTCGGGCTGGCCCGGGACGTGCACAACCTCGACTACTACA |
| | AGAAGACGACCAACGGCCGGCTGCCCGTGAAGTGGATGGCGCCTG |
| | AGGCCTTGTTTGACCGAGTCTACACTCACCAGAGTGACGTCTGGTC |
| | CTTTGGGGTCCTGCTCTGGGAGATCTTCACGCTGGGGGGCTCCCCG |
| | TACCCCGGCATCCCTGTGGAGGAGCTCTTCAAGCTGCTGAAGGAG |
| | GGCCACCGCATGGACAAGCCCGCCAACTGCACACACGACCTGTAC |
| | ATGATCATGCGGGAGTGCTGGCATGCCGCGCCCTCCCAGAGGCCC |
| | ACCTTCAAGCAGCTGGTGGAGGACCTGGACCGTGTCCTTACCGTG |
| | ACGTCCACCGACGTAAAGGCGACACAGGAGGAGAACCGGGAGCT |
| | GAGGAGCAGGTGTGAGGAGCTCCACGGGAAGAACCTGGAACTGG |
| | GGAAGATCATGGACAGGTTCGAAGAGGTTGTGTACCAGGCCATGG |
| | AGGAAGTTCAGAAGCAGAAGGAACTTTCCAAAGCTGAAATCCAGA |
| | AAGTTCTAAAAGAAAAAGACCAACTTACCACAGATCTGAACTCCA |
| | TGGAGAAGTCCTTCTCCGACCTCTTCAAGCGTTTTGAGAAACAGAA |
| | AGAGGTGATCGAGGGCTACCGCAAGAACGAAGAGTCACTGAAGA |
| | AGTGCGTGGAGGATTACCTGGCAAGGATCACCCAGGAGGGCCAGA |
| | GGTACCAAGCCCTGAAGGCCCACGCGGAGGAGAAGCTGCAGCTGG |
| | CAAACGAGGAGATCGCCCAGGTCCGGAGCAAGGCCCAGGCGGAA |
| | GCGTTGGCCCTCCAGGCCAGCCTGAGGAAGGAGCAGATGCGCATC |
| | CAGTCGCTGGAGAAGACAGTGGAGCAGAAGACTAAAGAGAACGA |
| | GGAGCTGACCAGGATCTGCGACGACCTCATCTCCAAGATGGAGAA |
| | GATCTGA |
| FGFR3-TACC3 | >ATGGGCGCCCCTGCCTGCGCCCTCGCGCTCTGCGTGGCCGTGGCC |
| V3 (2955 base | ATCGTGGCCGGCGCCTCCTCGGAGTCCTTGGGGACGGAGCAGCGC |
| pairs) | GTCGTGGGGCGAGCGGCAGAAGTCCCGGGCCCAGAGCCCGGCCAG |
| (SEQ ID NO: 3) | CAGGAGCAGTTGGTCTTCGGCAGCGGGGATGCTGTGGAGCTGAGC |
| | TGTCCCCCGCCCGGGGGTGGTCCCATGGGGCCCACTGTCTGGGTCA |
| | AGGATGGCACAGGGCTGGTGCCCTCGGAGCGTGTCCTGGTGGGGC |
| | CCCAGCGGCTGCAGGTGCTGAATGCCTCCCACGAGGACTCCGGGG |
| | CCTACAGCTGCCGGCAGCGGCTCACGCAGCGCGTACTGTGCCACTT |
| | CAGTGTGCGGGTGACAGACGCTCCATCCTCGGGAGATGACGAAGA |
| | CGGGGAGGACGAGGCTGAGGACACAGGTGTGGACACAGGGGCCC |
| | CTTACTGGACACGGCCCGAGCGGATGGACAAGAAGCTGCTGGCCG |
| | TGCCGGCCGCCAACACCGTCCGCTTCCGCTGCCCAGCCGCTGGCAA |
| | CCCCACTCCCTCCATCTCCTGGCTGAAGAACGGCAGGGAGTTCCGC |
| | GGCGAGCACCGCATTGGAGGCATCAAGCTGCGGCATCAGCAGTGG |
| | AGCCTGGTCATGGAAAGCGTGGTGCCCTCGGACCGCGGCAACTAC |
| | ACCTGCGTCGTGGAGAACAAGTTTGGCAGCATCCGGCAGACGTAC |
| | ACGCTGGACGTGCTGGAGCGCTCCCCGCACCGGCCCATCCTGCAG |
| | GCGGGGCTGCCGGCCAACCAGACGGCGGTGCTGGGCAGCGACGTG |
| | GAGTTCCACTGCAAGGTGTACAGTGACGCACAGCCCCACATCCAG |
| | TGGCTCAAGCACGTGGAGGTGAATGGCAGCAAGGTGGGCCCGGAC |
| | GGCACACCCTACGTTACCGTGCTCAAGACGGCGGGCGCTAACACC |
| | ACCGACAAGGAGCTAGAGGTTCTCTCCTTGCACAACGTCACCTTTG |
| | AGGACGCCGGGGAGTACACCTGCCTGGCGGGCAATTCTATTGGGT |
| | TTTCTCATCACTCTGCGTGGCTGGTGGTGCTGCCAGCCGAGGAGGA |
| | GCTGGTGGAGGCTGACGAGGCGGGCAGTGTGTATGCAGGCATCCT |
| | CAGCTACGGGGTGGGCTTCTTCCTGTTCATCCTGGTGGTGGCGGCT |
| | GTGACGCTCTGCCGCCTGCGCAGCCCCCCCAAGAAAGGCCTGGGC |
| | TCCCCCACCGTGCACAAGATCTCCCGCTTCCCGCTCAAGCGACAGG |
| | TGTCCCTGGAGTCCAACGCGTCCATGAGCTCCAACACACCACTGGT |
| | GCGCATCGCAAGGCTGTCCTCAGGGGAGGGCCCCACGCTGGCCAA |
| | TGTCTCCGAGCTCGAGCTGCCTGCCGACCCCAAATGGGAGCTGTCT |
| | CGGGCCCGGCTGACCCTGGGCAAGCCCCTTGGGGAGGGCTGCTTC |
| | GGCCAGGTGGTCATGGCGGAGGCCATCGGCATTGACAAGGACCGG |
| | GCCGCCAAGCCTGTCACCGTAGCCGTGAAGATGCTGAAAGACGAT |
| | GCCACTGACAAGGACCTGTCGGACCTGGTGTCTGAGATGGAGATG |
| | ATGAAGATGATCGGGAAACACAAAAACATCATCAACCTGCTGGGC |
| | GCCTGCACGCAGGGCGGGCCCCTGTACGTGCTGGTGGAGTACGCG |
| | GCCAAGGGTAACCTGCGGGAGTTTCTGCGGGCGCGGCGGCCCCCG |
| | GGCCTGGACTACTCCTTCGACACCTGCAAGCCGCCCGAGGAGCAG |
| | CTCACCTTCAAGGACCTGGTGTCCTGTGCCTACCAGGTGGCCCGGG |
| | GCATGGAGTACTTGGCCTCCCAGAAGTGCATCCACAGGGACCTGG |
| | CTGCCCGCAATGTGCTGGTGACCGAGGACAACGTGATGAAGATCG |
| | CAGACTTCGGGCTGGCCCGGGACGTGCACAACCTCGACTACTACA |
| | AGAAGACGACCAACGGCCGGCTGCCCGTGAAGTGGATGGCGCCTG |
| | AGGCCTTGTTTGACCGAGTCTACACTCACCAGAGTGACGTCTGGTC |
| | CTTTGGGGTCCTGCTCTGGGAGATCTTCACGCTGGGGGGCTCCCCG |
| | TACCCCGGCATCCCTGTGGAGGAGCTCTTCAAGCTGCTGAAGGAG |
| | GGCCACCGCATGGACAAGCCCGCCAACTGCACACACGACCTGTAC |
| | ATGATCATGCGGGAGTGCTGGCATGCCGCGCCCTCCCAGAGGCCC |
| | ACCTTCAAGCAGCTGGTGGAGGACCTGGACCGTGTCCTTACCGTG |
| | ACGTCCACCGACGTGCCAGGCCCACCCCCAGGTGTTCCCGCGCCTG |
| | GGGGCCCACCCCTGTCCACCGGACCTATAGTGGACCTGCTCCAGTA |
| | CAGCCAGAAGGACCTGGATGCAGTGGTAAAGGCGACACAGGAGG |
| | AGAACCGGGAGCTGAGGAGCAGGTGTGAGGAGCTCCACGGGAAG |
| | AACCTGGAACTGGGGAAGATCATGGACAGGTTCGAAGAGGTTGTG |
| | TACCAGGCCATGGAGGAAGTTCAGAAGCAGAAGGAACTTTCCAAA |
| | GCTGAAATCCAGAAAGTTCTAAAAGAAAAAGACCAACTTACCACA |
| | GATCTGAACTCCATGGAGAAGTCCTTCTCCGACCTCTTCAAGCGTT |
| | TTGAGAAACAGAAAGAGGTGATCGAGGGCTACCGCAAGAACGAA |
| | GAGTCACTGAAGAAGTGCGTGGAGGATTACCTGGCAAGGATCACC |
| | CAGGAGGGCCAGAGGTACCAAGCCCTGAAGGCCCACGCGGAGGA |
| | GAAGCTGCAGCTGGCAAACGAGGAGATCGCCCAGGTCCGGAGCA |
| | AGGCCCAGGCGGAAGCGTTGGCCCTCCAGGCCAGCCTGAGGAAGG |
| | AGCAGATGCGCATCCAGTCGCTGGAGAAGACAGTGGAGCAGAAG |
| | ACTAAAGAGAACGAGGAGCTGACCAGGATCTGCGACGACCTCATC |
| | TCCAAGATGGAGAAGATCTGA |
| FGFR3- | >ATGGGCGCCCCTGCCTGCGCCCTCGCGCTCTGCGTGGCCGTGGCC |
| BAIAP2L1 | ATCGTGGCCGGCGCCTCCTCGGAGTCCTTGGGGACGGAGCAGCGC |
| (3765 base | GTCGTGGGGCGAGCGGCAGAAGTCCCGGGCCCAGAGCCCGGCCAG |
| pairs) | CAGGAGCAGTTGGTCTTCGGCAGCGGGGATGCTGTGGAGCTGAGC |
| (SEQ ID NO: 4) | TGTCCCCCGCCCGGGGGTGGTCCCATGGGGCCCACTGTCTGGGTCA |
| | AGGATGGCACAGGGCTGGTGCCCTCGGAGCGTGTCCTGGTGGGGC |
| | CCCAGCGGCTGCAGGTGCTGAATGCCTCCCACGAGGACTCCGGGG |
| | CCTACAGCTGCCGGCAGCGGCTCACGCAGCGCGTACTGTGCCACTT |
| | CAGTGTGCGGGTGACAGACGCTCCATCCTCGGGAGATGACGAAGA |
| | CGGGGAGGACGAGGCTGAGGACACAGGTGTGGACACAGGGGCCC |
| | CTTACTGGACACGGCCCGAGCGGATGGACAAGAAGCTGCTGGCCG |
| | TGCCGGCCGCCAACACCGTCCGCTTCCGCTGCCCAGCCGCTGGCAA |
| | CCCCACTCCCTCCATCTCCTGGCTGAAGAACGGCAGGGAGTTCCGC |
| | GGCGAGCACCGCATTGGAGGCATCAAGCTGCGGCATCAGCAGTGG |
| | AGCCTGGTCATGGAAAGCGTGGTGCCCTCGGACCGCGGCAACTAC |
| | ACCTGCGTCGTGGAGAACAAGTTTGGCAGCATCCGGCAGACGTAC |
| | ACGCTGGACGTGCTGGAGCGCTCCCCGCACCGGCCCATCCTGCAG |
| | GCGGGGCTGCCGGCCAACCAGACGGCGGTGCTGGGCAGCGACGTG |
| | GAGTTCCACTGCAAGGTGTACAGTGACGCACAGCCCCACATCCAG |
| | TGGCTCAAGCACGTGGAGGTGAATGGCAGCAAGGTGGGCCCGGAC |
| | GGCACACCCTACGTTACCGTGCTCAAGTCCTGGATCAGTGAGAGT |
| | GTGGAGGCCGACGTGCGCCTCCGCCTGGCCAATGTGTCGGAGCGG |
| | GACGGGGGCGAGTACCTCTGTCGAGCCACCAATTTCATAGGCGTG |
| | GCCGAGAAGGCCTTTTGGCTGAGCGTTCACGGGCCCCGAGCAGCC |
| | GAGGAGGAGCTGGTGGAGGCTGACGAGGCGGGCAGTGTGTATGC |
| | AGGCATCCTCAGCTACGGGGGGGCTTCTTCCTGTTCATCCTGGTG |
| | GTGGCGGCTGTGACGCTCTGCCGCCTGCGCAGCCCCCCCAAGAAA |
| | GGCCTGGGCTCCCCCACCGTGCACAAGATCTCCCGCTTCCCGCTCA |
| | AGCGACAGGTGTCCCTGGAGTCCAACGCGTCCATGAGCTCCAACA |
| | CACCACTGGTGCGCATCGCAAGGCTGTCCTCAGGGGAGGGCCCCA |
| | CGCTGGCCAATGTCTCCGAGCTCGAGCTGCCTGCCGACCCCAAATG |
| | GGAGCTGTCTCGGGCCCGGCTGACCCTGGGCAAGCCCCTTGGGGA |
| | GGGCTGCTTCGGCCAGGTGGTCATGGCGGAGGCCATCGGCATTGA |
| | CAAGGACCGGGCCGCCAAGCCTGTCACCGTAGCCGTGAAGATGCT |
| | GAAAGACGATGCCACTGACAAGGACCTGTCGGACCTGGTGTCTGA |
| | GATGGAGATGATGAAGATGATCGGGAAACACAAAAACATCATCA |
| | ACCTGCTGGGCGCCTGCACGCAGGGCGGGCCCCTGTACGTGCTGG |
| | TGGAGTACGCGGCCAAGGGTAACCTGCGGGAGTTTCTGCGGGCGC |
| | GGCGGCCCCCGGGCCTGGACTACTCCTTCGACACCTGCAAGCCGC |
| | CCGAGGAGCAGCTCACCTTCAAGGACCTGGTGTCCTGTGCCTACCA |
| | GGTGGCCCGGGGCATGGAGTACTTGGCCTCCCAGAAGTGCATCCA |
| | CAGGGACCTGGCTGCCCGCAATGTGCTGGTGACCGAGGACAACGT |
| | GATGAAGATCGCAGACTTCGGGCTGGCCCGGGACGTGCACAACCT |
| | CGACTACTACAAGAAGACGACCAACGGCCGGCTGCCCGTGAAGTG |
| | GATGGCGCCTGAGGCCTTGTTTGACCGAGTCTACACTCACCAGAGT |
| | GACGTCTGGTCCTTTGGGGTCCTGCTCTGGGAGATCTTCACGCTGG |
| | GGGGCTCCCCGTACCCCGGCATCCCTGTGGAGGAGCTCTTCAAGCT |
| | GCTGAAGGAGGGCCACCGCATGGACAAGCCCGCCAACTGCACACA |
| | CGACCTGTACATGATCATGCGGGAGTGCTGGCATGCCGCGCCCTCC |
| | CAGAGGCCCACCTTCAAGCAGCTGGTGGAGGACCTGGACCGTGTC |
| | CTTACCGTGACGTCCACCGACAATGTTATGGAACAGTTCAATCCTG |
| | GGCTGCGAAATTTAATAAACCTGGGGAAAAATTATGAGAAAGCTG |
| | TAAACGCTATGATCCTGGCAGGAAAAGCCTACTACGATGGAGTGG |
| | CCAAGATCGGTGAGATTGCCACTGGGTCCCCCGTGTCAACTGAACT |
| | GGGACATGTCCTCATAGAGATTTCAAGTACCCACAAGAAACTCAA |
| | CGAGAGTCTTGATGAAAATTTTAAAAAATTCCACAAAGAGATTAT |
| | CCATGAGCTGGAGAAGAAGATAGAACTTGACGTGAAATATATGAA |
| | CGCAACTCTAAAAAGATACCAAACAGAACACAAGAATAAATTAGA |
| | GTCTTTGGAGAAATCCCAAGCTGAGTTGAAGAAGATCAGAAGGAA |
| | AAGCCAAGGAAGCCGAAACGCACTCAAATATGAACACAAAGAAA |
| | TTGAGTATGTGGAGACCGTTACTTCTCGTCAGAGTGAAATCCAGAA |
| | ATTCATTGCAGATGGTTGCAAAGAGGCTCTGCTTGAAGAGAAGAG |
| | GCGCTTCTGCTTTCTGGTTGATAAGCACTGTGGCTTTGCAAACCAC |
| | ATACATTATTATCACTTACAGTCTGCAGAACTACTGAATTCCAAGC |
| | TGCCTCGGTGGCAGGAGACCTGTGTTGATGCCATCAAAGTGCCAG |
| | AGAAAATCATGAATATGATCGAAGAAATAAAGACCCCAGCCTCTA |
| | CCCCCGTGTCTGGAACTCCTCAGGCTTCACCCATGATCGAGAGAAG |
| | CAATGTGGTTAGGAAAGATTACGACACCCTTTCTAAATGCTCACCA |
| | AAGATGCCCCCCGCTCCTTCAGGCAGAGCATATACCAGTCCCTTGA |
| | TCGATATGTTTAATAACCCAGCCACGGCTGCCCCGAATTCACAAAG |
| | GGTAAATAATTCAACAGGTACTTCCGAAGATCCCAGTTTACAGCG |
| | ATCAGTTTCGGTTGCAACGGGACTGAACATGATGAAGAAGCAGAA |
| | AGTGAAGACCATCTTCCCGCACACTGCGGGCTCCAACAAGACCTT |
| | ACTCAGCTTTGCACAGGGAGATGTCATCACGCTGCTCATCCCCGAG |
| | GAGAAGGATGGCTGGCTCTATGGAGAACACGACGTGTCCAAGGCG |
| | AGGGGTTGGTTCCCGTCGTCGTACACGAAGTTGCTGGAAGAAAAT |
| | GAGACAGAAGCAGTGACCGTGCCCACGCCAAGCCCCACACCAGTG |
| | AGAAGCATCAGCACCGTGAACTTGTCTGAGAATAGCAGTGTTGTC |
| | ATCCCCCCACCCGACTACTTGGAATGCTTGTCCATGGGGGCAGCTG |
| | CCGACAGGAGAGCAGATTCGGCCAGGACGACATCCACCTTTAAGG |
| | CCCCAGCGTCCAAGCCCGAGACCGCGGCTCCTAACGATGCCAACG |
| | GGACTGCAAAGCCGCCTTTTCTCAGCGGAGAAAACCCCTTTGCCAC |
| | TGTGAAACTCCGCCCGACTGTGACGAATGATCGCTCGGCACCCATC |
| | ATTCGATGA |
| FGFR2-BICC1 | >ATGGTCAGCTGGGGTCGTTTCATCTGCCTGGTCGTGGTCACCATG |
| (4989 base | GCAACCTTGTCCCTGGCCCGGCCCTCCTTCAGTTTAGTTGAGGATA |
| pairs) | CCACATTAGAGCCAGAAGAGCCACCAACCAAATACCAAATCTCTC |
| (SEQ ID NO: 5) | AACCAGAAGTGTACGTGGCTGCGCCAGGGGAGTCGCTAGAGGTGC |
| | GCTGCCTGTTGAAAGATGCCGCCGTGATCAGTTGGACTAAGGATG |
| | GGGTGCACTTGGGGCCCAACAATAGGACAGTGCTTATTGGGGAGT |
| | ACTTGCAGATAAAGGGCGCCACGCCTAGAGACTCCGGCCTCTATG |
| | CTTGTACTGCCAGTAGGACTGTAGACAGTGAAACTTGGTACTTCAT |
| | GGTGAATGTCACAGATGCCATCTCATCCGGAGATGATGAGGATGA |
| | CACCGATGGTGCGGAAGATTTTGTCAGTGAGAACAGTAACAACAA |
| | GAGAGCACCATACTGGACCAACACAGAAAAGATGGAAAAGCGGC |
| | TCCATGCTGTGCCTGCGGCCAACACTGTCAAGTTTCGCTGCCCAGC |
| | CGGGGGGAACCCAATGCCAACCATGCGGTGGCTGAAAAACGGGA |
| | AGGAGTTTAAGCAGGAGCATCGCATTGGAGGCTACAAGGTACGAA |
| | ACCAGCACTGGAGCCTCATTATGGAAAGTGTGGTCCCATCTGACA |
| | AGGGAAATTATACCTGTGTAGTGGAGAATGAATACGGGTCCATCA |
| | ATCACACGTACCACCTGGATGTTGTGGAGCGATCGCCTCACCGGCC |
| | CATCCTCCAAGCCGGACTGCCGGCAAATGCCTCCACAGTGGTCGG |
| | AGGAGACGTAGAGTTTGTCTGCAAGGTTTACAGTGATGCCCAGCC |
| | CCACATCCAGTGGATCAAGCACGTGGAAAAGAACGGCAGTAAATA |
| | CGGGCCCGACGGGCTGCCCTACCTCAAGGTTCTCAAGGCCGCCGG |
| | TGTTAACACCACGGACAAAGAGATTGAGGTTCTCTATATTCGGAAT |
| | GTAACTTTTGAGGACGCTGGGGAATATACGTGCTTGGCGGGTAATT |
| | CTATTGGGATATCCTTTCACTCTGCATGGTTGACAGTTCTGCCAGC |
| | GCCTGGAAGAGAAAAGGAGATTACAGCTTCCCCAGACTACCTGGA |
| | GATAGCCATTTACTGCATAGGGGTCTTCTTAATCGCCTGTATGGTG |
| | GTAACAGTCATCCTGTGCCGAATGAAGAACACGACCAAGAAGCCA |
| | GACTTCAGCAGCCAGCCGGCTGTGCACAAGCTGACCAAACGTATC |
| | CCCCTGCGGAGACAGGTAACAGTTTCGGCTGAGTCCAGCTCCTCCA |
| | TGAACTCCAACACCCCGCTGGTGAGGATAACAACACGCCTCTCTTC |
| | AACGGCAGACACCCCCATGCTGGCAGGGGTCTCCGAGTATGAACT |
| | TCCAGAGGACCCAAAATGGGAGTTTCCAAGAGATAAGCTGACACT |
| | GGGCAAGCCCCTGGGAGAAGGTTGCTTTGGGCAAGTGGTCATGGC |
| | GGAAGCAGTGGGAATTGACAAAGACAAGCCCAAGGAGGCGGTCA |
| | CCGTGGCCGTGAAGATGTTGAAAGATGATGCCACAGAGAAAGACC |
| | TTTCTGATCTGGTGTCAGAGATGGAGATGATGAAGATGATTGGGA |
| | AACACAAGAATATCATAAATCTTCTTGGAGCCTGCACACAGGATG |
| | GGCCTCTCTATGTCATAGTTGAGTATGCCTCTAAAGGCAACCTCCG |
| | AGAATACCTCCGAGCCCGGAGGCCACCCGGGATGGAGTACTCCTA |
| | TGACATTAACCGTGTTCCTGAGGAGCAGATGACCTTCAAGGACTTG |
| | GTGTCATGCACCTACCAGCTGGCCAGAGGCATGGAGTACTTGGCTT |
| | CCCAAAAATGTATTCATCGAGATTTAGCAGCCAGAAATGTTTTGGT |
| | AACAGAAAACAATGTGATGAAAATAGCAGACTTTGGACTCGCCAG |
| | AGATATCAACAATATAGACTATTACAAAAAGACCACCAATGGGCG |
| | GCTTCCAGTCAAGTGGATGGCTCCAGAAGCCCTGTTTGATAGAGTA |
| | TACACTCATCAGAGTGATGTCTGGTCCTTCGGGGTGTTAATGTGGG |
| | AGATCTTCACTTTAGGGGGCTCGCCCTACCCAGGGATTCCCGTGGA |
| | GGAACTTTTTAAGCTGCTGAAGGAAGGACACAGAATGGATAAGCC |
| | AGCCAACTGCACCAACGAACTGTACATGATGATGAGGGACTGTTG |
| | GCATGCAGTGCCCTCCCAGAGACCAACGTTCAAGCAGTTGGTAGA |
| | AGACTTGGATCGAATTCTCACTCTCACAACCAATGAGATCATGGA |
| | GGAAACAAATACGCAGATTGCTTGGCCATCAAAACTGAAGATCGG |
| | AGCCAAATCCAAGAAAGATCCCCATATTAAGGTTTCTGGAAAGAA |
| | AGAAGATGTTAAAGAAGCCAAGGAAATGATCATGTCTGTCTTAGA |
| | CACAAAAAGCAATCGAGTCACACTGAAGATGGATGTTTCACATAC |
| | AGAACATTCACATGTAATCGGCAAAGGTGGCAACAATATTAAAAA |
| | AGTGATGGAAGAAACCGGATGCCATATCCACTTTCCAGATTCCAA |
| | CAGGAATAACCAAGCAGAAAAAAGCAACCAGGTATCTATAGCGG |
| | GACAACCAGCAGGAGTAGAATCTGCCCGAGTTAGAATTCGGGAGC |
| | TGCTTCCTTTGGTGCTGATGTTTGAGCTACCAATTGCTGGAATTCTT |
| | CAACCGGTTCCTGATCCTAATTCCCCCTCTATTCAGCATATATCAC |
| | AAACGTACAATATTTCAGTATCATTTAAACAGCGTTCCCGAATGTA |
| | TGGTGCTACTGTCATAGTACGAGGGTCTCAGAATAACACTAGTGCT |
| | GTGAAGGAAGGAACTGCCATGCTGTTAGAACATCTTGCTGGGAGC |
| | TTAGCATCAGCTATTCCTGTGAGCACACAACTAGATATTGCAGCTC |
| | AACATCATCTCTTTATGATGGGTCGAAATGGGAGCAACATCAAAC |
| | ATATCATGCAGAGAACAGGTGCTCAGATCCACTTTCCTGATCCCAG |
| | TAATCCACAAAAGAAATCTACCGTCTACCTCCAGGGCACCATTGA |
| | GTCTGTCTGTCTTGCAAGGCAATATCTCATGGGTTGTCTTCCTCTTG |
| | TGTTGATGTTTGATATGAAGGAAGAAATTGAAGTAGATCCACAAT |
| | TCATTGCGCAGTTGATGGAACAGCTTGATGTCTTCATCAGTATTAA |
| | ACCAAAGCCCAAACAGCCAAGCAAGTCTGTGATTGTGAAAAGTGT |
| | TGAGCGAAATGCCTTAAATATGTATGAAGCAAGGAAATGTCTCCT |
| | CGGACTTGAAAGCAGTGGGGTTACCATAGCAACCAGTCCATCCCC |
| | AGCATCCTGCCCTGCCGGCCTGGCATGTCCCAGCCTGGATATCTTA |
| | GCTTCAGCAGGCCTTGGACTCACTGGACTAGGTCTTTTGGGACCCA |
| | CCACCTTATCTCTGAACACTTCAACAACCCCAAACTCACTCTTGAA |
| | TGCTCTTAATAGCTCAGTCAGTCCTTTGCAAAGTCCAAGTTCTGGT |
| | ACACCCAGCCCCACATTATGGGCACCCCCACTTGCTAATACTTCAA |
| | GTGCCACAGGTTTTTCTGCTATACCACACCTTATGATTCCATCTACT |
| | GCCCAAGCCACATTAACTAATATTTTGTTGTCTGGAGTGCCCACCT |
| | ATGGGCACACAGCTCCATCTCCCCCTCCTGGCTTGACTCCTGTTGA |
| | TGTCCATATCAACAGTATGCAGACCGAAGGCAAAAAAATCTCTGC |
| | TGCTTTAAATGGACATGCACAGTCTCCAGATATAAAATATGGTGCA |
| | ATATCCACTTCATCACTTGGAGAAAAAGTGCTGAGTGCAAATCAC |
| | GGGGATCCGTCCATCCAGACAAGTGGGTCTGAGCAGACATCTCCC |
| | AAATCAAGCCCCACTGAAGGTTGTAATGATGCTTTTGTTGAAGTAG |
| | GCATGCCTCGAAGTCCTTCCCATTCTGGGAATGCTGGTGACTTGAA |
| | ACAGATGATGTGTCCCTCCAAGGTTTCCTGTGCCAAAAGGCAGAC |
| | AGTGGAACTATTGCAAGGCACGAAAAACTCACACTTACACAGCAC |
| | TGACAGGTTGCTCTCAGACCCTGAACTGAGTGCTACCGAAAGCCCT |
| | TTGGCTGACAAGAAGGCTCCAGGGAGTGAGCGCGCTGCAGAGAGG |
| | GCAGCAGCTGCCCAGCAAAACTCCGAAAGGGCCCACCTTGCTCCA |
| | CGGTCATCATATGTCAACATGCAGGCATTTGACTATGAACAGAAG |
| | AAGCTATTAGCCACCAAAGCTATGTTAAAGAAACCAGTGGTGACG |
| | GAGGTCAGAACGCCCACAAATACCTGGAGTGGCCTGGGTTTTTCT |
| | AAATCCATGCCAGCTGAAACTATCAAGGAGTTGAGAAGGGCCAAT |
| | CATGTGTCCTATAAGCCCACAATGACAACCACTTATGAGGGCTCAT |
| | CCATGTCCCTTTCACGGTCCAACAGTCGTGAGCACTTGGGAGGTGG |
| | AAGCGAATCTGATAACTGGAGAGACCGAAATGGAATTGGACCTGG |
| | AAGTCATAGTGAATTTGCAGCTTCTATTGGCAGCCCTAAGCGTAAA |
| | CAAAACAAATCAACGGAACACTATCTCAGCAGTAGCAATTACATG |
| | GACTGCATTTCCTCGCTGACAGGAAGCAATGGCTGTAACTTAAATA |
| | GCTCTTTCAAAGGTTCTGACCTCCCTGAGCTCTTCAGCAAACTGGG |
| | CCTGGGCAAATACACAGATGTTTTCCAGCAACAAGAGATCGATCT |
| | TCAGACATTCCTCACTCTCACAGATCAGGATCTGAAGGAGCTGGG |
| | AATAACTACTTTTGGTGCCAGGAGGAAAATGCTGCTTGCAATTTCA |
| | GAACTAAATAAAAACCGAAGAAAGCTTTTTGAATCGCCAAATGCA |
| | CGCACCTCTTTCCTGGAAGGTGGAGCGAGTGGAAGGCTACCCCGT |
| | CAGTATCACTCAGACATTGCTAGTGTCAGTGGCCGCTGGTAG |
| FGFR2-CASP7 | >ATGGTCAGCTGGGGTCGTTTCATCTGCCTGGTCGTGGTCACCATG |
| (3213 base | GCAACCTTGTCCCTGGCCCGGCCCTCCTTCAGTTTAGTTGAGGATA |
| pairs) | CCACATTAGAGCCAGAAGAGCCACCAACCAAATACCAAATCTCTC |
| (SEQ ID NO: 6) | AACCAGAAGTGTACGTGGCTGCGCCAGGGGAGTCGCTAGAGGTGC |
| | GCTGCCTGTTGAAAGATGCCGCCGTGATCAGTTGGACTAAGGATG |
| | GGGTGCACTTGGGGCCCAACAATAGGACAGTGCTTATTGGGGAGT |
| | ACTTGCAGATAAAGGGCGCCACGCCTAGAGACTCCGGCCTCTATG |
| | CTTGTACTGCCAGTAGGACTGTAGACAGTGAAACTTGGTACTTCAT |
| | GGTGAATGTCACAGATGCCATCTCATCCGGAGATGATGAGGATGA |
| | CACCGATGGTGCGGAAGATTTTGTCAGTGAGAACAGTAACAACAA |
| | GAGAGCACCATACTGGACCAACACAGAAAAGATGGAAAAGCGGC |
| | TCCATGCTGTGCCTGCGGCCAACACTGTCAAGTTTCGCTGCCCAGC |
| | CGGGGGGAACCCAATGCCAACCATGCGGTGGCTGAAAAACGGGA |
| | AGGAGTTTAAGCAGGAGCATCGCATTGGAGGCTACAAGGTACGAA |
| | ACCAGCACTGGAGCCTCATTATGGAAAGTGTGGTCCCATCTGACA |
| | AGGGAAATTATACCTGTGTAGTGGAGAATGAATACGGGTCCATCA |
| | ATCACACGTACCACCTGGATGTTGTGGAGCGATCGCCTCACCGGCC |
| | CATCCTCCAAGCCGGACTGCCGGCAAATGCCTCCACAGTGGTCGG |
| | AGGAGACGTAGAGTTTGTCTGCAAGGTTTACAGTGATGCCCAGCC |
| | CCACATCCAGTGGATCAAGCACGTGGAAAAGAACGGCAGTAAATA |
| | CGGGCCCGACGGGCTGCCCTACCTCAAGGTTCTCAAGGCCGCCGG |
| | TGTTAACACCACGGACAAAGAGATTGAGGTTCTCTATATTCGGAAT |
| | GTAACTTTTGAGGACGCTGGGGAATATACGTGCTTGGCGGGTAATT |
| | CTATTGGGATATCCTTTCACTCTGCATGGTTGACAGTTCTGCCAGC |
| | GCCTGGAAGAGAAAAGGAGATTACAGCTTCCCCAGACTACCTGGA |
| | GATAGCCATTTACTGCATAGGGGTCTTCTTAATCGCCTGTATGGTG |
| | GTAACAGTCATCCTGTGCCGAATGAAGAACACGACCAAGAAGCCA |
| | GACTTCAGCAGCCAGCCGGCTGTGCACAAGCTGACCAAACGTATC |
| | CCCCTGCGGAGACAGGTAACAGTTTCGGCTGAGTCCAGCTCCTCCA |
| | TGAACTCCAACACCCCGCTGGTGAGGATAACAACACGCCTCTCTTC |
| | AACGGCAGACACCCCCATGCTGGCAGGGGTCTCCGAGTATGAACT |
| | TCCAGAGGACCCAAAATGGGAGTTTCCAAGAGATAAGCTGACACT |
| | GGGCAAGCCCCTGGGAGAAGGTTGCTTTGGGCAAGTGGTCATGGC |
| | GGAAGCAGTGGGAATTGACAAAGACAAGCCCAAGGAGGCGGTCA |
| | CCGTGGCCGTGAAGATGTTGAAAGATGATGCCACAGAGAAAGACC |
| | TTTCTGATCTGGTGTCAGAGATGGAGATGATGAAGATGATTGGGA |
| | AACACAAGAATATCATAAATCTTCTTGGAGCCTGCACACAGGATG |
| | GGCCTCTCTATGTCATAGTTGAGTATGCCTCTAAAGGCAACCTCCG |
| | AGAATACCTCCGAGCCCGGAGGCCACCCGGGATGGAGTACTCCTA |
| | TGACATTAACCGTGTTCCTGAGGAGCAGATGACCTTCAAGGACTTG |
| | GTGTCATGCACCTACCAGCTGGCCAGAGGCATGGAGTACTTGGCTT |
| | CCCAAAAATGTATTCATCGAGATTTAGCAGCCAGAAATGTTTTGGT |
| | AACAGAAAACAATGTGATGAAAATAGCAGACTTTGGACTCGCCAG |
| | AGATATCAACAATATAGACTATTACAAAAAGACCACCAATGGGCG |
| | GCTTCCAGTCAAGTGGATGGCTCCAGAAGCCCTGTTTGATAGAGTA |
| | TACACTCATCAGAGTGATGTCTGGTCCTTCGGGGTGTTAATGTGGG |
| | AGATCTTCACTTTAGGGGGCTCGCCCTACCCAGGGATTCCCGTGGA |
| | GGAACTTTTTAAGCTGCTGAAGGAAGGACACAGAATGGATAAGCC |
| | AGCCAACTGCACCAACGAACTGTACATGATGATGAGGGACTGTTG |
| | GCATGCAGTGCCCTCCCAGAGACCAACGTTCAAGCAGTTGGTAGA |
| | AGACTTGGATCGAATTCTCACTCTCACAACCAATGAGATGGCAGA |
| | TGATCAGGGCTGTATTGAAGAGCAGGGGGTTGAGGATTCAGCAAA |
| | TGAAGATTCAGTGGATGCTAAGCCAGACCGGTCCTCGTTTGTACCG |
| | TCCCTCTTCAGTAAGAAGAAGAAAAATGTCACCATGCGATCCATC |
| | AAGACCACCCGGGACCGAGTGCCTACATATCAGTACAACATGAAT |
| | TTTGAAAAGCTGGGCAAATGCATCATAATAAACAACAAGAACTTT |
| | GATAAAGTGACAGGTATGGGCGTTCGAAACGGAACAGACAAAGA |
| | TGCCGAGGCGCTCTTCAAGTGCTTCCGAAGCCTGGGTTTTGACGTG |
| | ATTGTCTATAATGACTGCTCTTGTGCCAAGATGCAAGATCTGCTTA |
| | AAAAAGCTTCTGAAGAGGACCATACAAATGCCGCCTGCTTCGCCT |
| | GCATCCTCTTAAGCCATGGAGAAGAAAATGTAATTTATGGGAAAG |
| | ATGGTGTCACACCAATAAAGGATTTGACAGCCCACTTTAGGGGGG |
| | ATAGATGCAAAACCCTTTTAGAGAAACCCAAACTCTTCTTCATTCA |
| | GGCTTGCCGAGGGACCGAGCTTGATGATGGCATCCAGGCCGACTC |
| | GGGGCCCATCAATGACACAGATGCTAATCCTCGATACAAGATCCC |
| | AGTGGAAGCTGACTTCCTCTTCGCCTATTCCACGGTTCCAGGCTAT |
| | TACTCGTGGAGGAGCCCAGGAAGAGGCTCCTGGTTTGTGCAAGCC |
| | CTCTGCTCCATCCTGGAGGAGCACGGAAAAGACCTGGAAATCATG |
| | CAGATCCTCACCAGGGTGAATGACAGAGTTGCCAGGCACTTTGAG |
| | TCTCAGTCTGATGACCCACACTTCCATGAGAAGAAGCAGATCCCCT |
| | GTGTGGTCTCCATGCTCACCAAGGAACTCTACTTCAGTCAATAG |

FGFR genetic alterations include FGFR single nucleotide polymorphism (SNP). “FGFR single nucleotide polymorphism” (SNP) refers to a FGFR2 or FGFR3 gene in which a single nucleotide differs among individuals. In certain embodiments, the FGFR2 or FGFR3 genetic alteration is an FGFR3 gene mutation. In particular, FGFR single nucleotide polymorphism” (SNP) refers to a FGFR3 gene in which a single nucleotide differs among individuals. The presence of one or more of the following FGFR SNPs in a biological sample from a patient can be determined by methods known to those of ordinary skill in the art or methods disclosed in WO 2016/048833: FGFR3 R248C, FGFR3 S249C, FGFR3 G370C, FGFR3 Y373C, or any combination thereof. The sequences of the FGFR SNPs are provided in Table 4A.

TABLE 4A

| FGFR3 mutant | Sequence |
|---|---|
| FGFR3 R248C | TCGGACCGCGGCAACTACACCTGCGTCGTGGAGAACAAGTTTGGC |
| | AGCATCCGGCAGACGTACACGCTGGACGTGCTGGAG(T)GCTCCCC |
| | GCACCGGCCCATCCTGCAGGCGGGGCTGCCGGCCAACCAGACGGC |
| | GGTGCTGGGCAGCGACGTGGAGTTCCACTGCAAGGTGTACAGTGA |
| | CGCACAGCCCCACATCCAGTGGCTCAAGCACGTGGAGGTGAATGG |
| | CAGCAAGGTGGGCCCGGACGGCACACCCTACGTTACCGTGCTCA |
| | (SEQ ID NO: 7) |
| FGFR3 S249C | GACCGCGGCAACTACACCTGCGTCGTGGAGAACAAGTTTGGCAGC |
| | ATCCGGCAGACGTACACGCTGGACGTGCTGGGTGAGGGCCCTGGG |
| | GCGGCGCGGGGGTGGGGGCGGCAGTGGCGGTGGTGGTGAGGGAG |
| | GGGGTGGCCCCTGAGCGTCATCTGCCCCCACAGAGCGCT(G)CCCG |
| | CACCGGCCCATCCTGCAGGCGGGGCTGCCGGCCAACCAGACGGCG |
| | GTGCTGGGCAGCGACGTGGAGTTCCACTGCAAGGTGTACAGTGAC |
| | GCACAGCCCCACATCCAGTGGCTCAAGCACGTGGAGGTGAATGGC |
| | AGCAAGGTGGGCCCGGACGGCACACCCTACGTTACCGTGCTCAAG |
| | GTGGGCCACCGTGTGCACGT |
| | (SEQ ID NO: 8) |
| FGFR3 G370C | GCGGGCAATTCTATTGGGTTTTCTCATCACTCTGCGTGGCTGGTGG |
| | TGCTGCCAGCCGAGGAGGAGCTGGTGGAGGCTGACGAGGCG(T)GC |
| | AGTGTGTATGCAGGCATCCTCAGCTACGGGGTGGGCTTCTTCCTGT |
| | TCATCCTGGTGGTGGCGGCTGTGACGCTCTGCCGCCTGCGCAGCCC |
| | CCCCAAGAAAGGCCTGGGCTCCCCCACCGTGCACAAGATCTCCCG |
| | CTTCCCG |
| | (SEQ ID NO: 9) |
| FGFR3 Y373C* | CTAGAGGTTCTCTCCTTGCACAACGTCACCTTTGAGGACGCCGGGG |
| | AGTACACCTGCCTGGCGGGCAATTCTATTGGGTTTTCTCATCACTC |
| | TGCGTGGCTGGTGGTGCTGCCAGCCGAGGAGGAGCTGGTGGAGGC |
| | TGACGAGGCGGGCAGTGTGT(G)TGCAGGCATCCTCAGCTACGGGG |
| | TGGGCTTCTTCCTGTTCATCCTGGTGGTGGCGGCTGTGACGCTCTG |
| | CCGCCTGCGCAGCCCCCCCAAGAAAGGCCTGGGCTCCCCCACCGT |
| | GCACAAGATCTCCCGCTTCCCGCTCAAGC |
| | (SEQ ID NO: 10) |

Sequences correspond to nucleotides 920-1510 of FGFR3 (Genebank ID # NM_000142.4).
Nucleotides in bold underline represent the SNP.
*Sometimes mistakenly referred to as Y375C in the literature.

As used herein, “FGFR genetic alteration gene panel” includes one or more of the above listed FGFR genetic alterations.

The FGFR genetic alteration gene panel that is used in the evaluating step of the disclosed methods for patients with urothelial carcinoma, in particular locally advanced or metastatic UC, comprises FGFR3-TACC3 V1, FGFR3-TACC3 V3, FGFR3-BAIAP2L1, FGFR2-BICC1, FGFR2-CASP7, FGFR3 R248C, FGFR3 S249C, FGFR3 G370C, or FGFR3 Y373C, or any combination thereof.

In an embodiment, the one or more FGFR genetic alterations as described herein comprise FGFR3-TACC3 V1, FGFR3-TACC3 V3, FGFR3-BAIAP2L1, FGFR2-BICC1, FGFR2-CASP7, FGFR3 R248C, FGFR3 S249C, FGFR3 G370C, or FGFR3 Y373C, or any combination thereof. In an embodiment, these one or more FGFR genetic alterations are considered susceptible FGFR genetic alterations.

In an embodiment, the one or more FGFR genetic alterations as described herein comprise a FGFR3 genetic alteration, in particular the one or more FGFR genetic alterations comprise FGFR3 FGFR3-TACC3 V1, FGFR3-TACC3 V3, FGFR3-BAIAP2L1, FGFR3 R248C, FGFR3 S249C, FGFR3 G370C, or FGFR3 Y373C, or any combination thereof. In an embodiment, these one or more FGFR genetic alterations are considered susceptible FGFR genetic alterations.

In an embodiment, the one or more FGFR genetic alterations as described herein comprise FGFR3-TACC3 V1, FGFR3-TACC3 V3, or any combination thereof. In an embodiment, these one or more FGFR genetic alterations are considered susceptible FGFR genetic alterations.

In an embodiment, the one or more FGFR genetic alterations as described herein comprise FGFR3 R248C, FGFR3 S249C, FGFR3 G370C, or FGFR3 Y373C, or any combination thereof. In an embodiment, these one or more FGFR genetic alterations are considered susceptible FGFR genetic alterations.

Provided herein are methods of treating subjects with advanced urothelial cancer harboring one or more FGFR alterations who have progressed or whose urothelial cancer harboring one or more FGFR alterations has progressed after 1 or 2 prior treatments, at least 1 of which includes an anti-programmed death-[ligand]1(PD-[L]1) agent comprising administering to the subjects a therapeutically effective amount of erdafitinib, said method providing improved treatment for the subjects versus chemotherapy.

### SUMMARY OF THE INVENTION

In an aspect there is provided a method of treating locally advanced or metastatic urothelial carcinoma (mUC) harboring one or more fibroblast growth factor receptor (FGFR) genetic alterations, comprising administering to a subject in need thereof, in particular a patient with locally advanced or metastatic urothelial carcinoma, more in particular an adult patient with locally advanced or metastatic urothelial carcinoma, a therapeutically effective amount of erdafitinib, wherein the subject, in particular the patient, more in particular the adult patient, received at least one line of systemic treatment containing an anti-PD-(L)1 agent in the locally advanced or metastatic treatment. In certain embodiments, the locally advanced or metastatic UC (urothelial carcinoma) is upper tract UC.

In an aspect, there is provided a method of treating locally advanced or metastatic urothelial carcinoma (mUC), harboring one or more fibroblast growth factor receptor (FGFR) genetic alterations, comprising administering to a subject in need thereof, in particular a patient with locally advanced or metastatic urothelial carcinoma, more in particular an adult patient with locally advanced or metastatic urothelial carcinoma, a therapeutically effective amount of erdafitinib, wherein the subject, in particular the patient, more in particular the adult patient, progressed on or after ≥1 prior treatment that included an anti-PD-(L)1 agent. In certain embodiments, the locally advanced or metastatic UC is upper tract UC.

In an aspect, there is provided a method of treating locally advanced or metastatic urothelial carcinoma (mUC) harboring one or more FGFR genetic alterations, comprising administering to a subject in need thereof, in particular a patient with locally advanced or metastatic urothelial carcinoma, more in particular an adult patient with locally advanced or metastatic urothelial carcinoma, a therapeutically effective amount of erdafitinib, wherein the subject, in particular the patient, more in particular the adult patient, progressed during or following at least one line of prior treatment with an anti-PD-(L)1 agent including within 12 months of neoadjuvant or adjuvant therapy. In certain embodiments, the locally advanced or metastatic UC is upper tract UC.

In an aspect, there is provided a method of treating locally advanced or metastatic urothelial carcinoma (mUC) harboring one or more FGFR genetic alterations, comprising administering to a subject in need thereof, in particular a patient with locally advanced or metastatic urothelial carcinoma, more in particular an adult patient with locally advanced or metastatic urothelial carcinoma, a therapeutically effective amount of erdafitinib, wherein the locally advanced or metastatic urothelial carcinoma or the subject, in particular the patient, more in particular the adult patient, progressed during or following at least one line of prior treatment with an anti-PD-(L)1 agent in a neoadjuvant or adjuvant setting. In certain embodiments, the locally advanced or metastatic UC is upper tract UC.

In an aspect, there is provided a method for the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who has received at least one line of systemic treatment containing an anti-PD-(L)1 agent in the locally advanced or metastatic treatment setting, comprising administering to the subject, in particular the patient, more in particular the adult patient, a therapeutically effective amount of erdafitinib. In certain embodiments, the locally advanced or metastatic UC is upper tract UC.

In an aspect, there is provided a method for the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who progressed on or after ≥1 prior treatment that included an anti-PD-(L)1 agent, comprising administering to the subject, in particular the patient, more in particular the adult patient, a therapeutically effective amount of erdafitinib. In certain embodiments, the locally advanced or metastatic UC is upper tract UC.

In an aspect, there is provided a method for the treatment of a subject in need thereof, in particular a patient with locally advanced or metastatic urothelial carcinoma, more in particular an adult patient with locally advanced or metastatic urothelial carcinoma, that has
one or more FGFR genetic alterations and
progressed during or following at least one line of prior treatment with an anti-PD-(L)1 agent including within 12 months of neoadjuvant or adjuvant therapy, comprising administering to the subject, in particular the patient, more in particular the adult patient, a therapeutically effective amount of erdafitinib. In certain embodiments, the locally advanced or metastatic UC is upper tract UC.

In an aspect, there is provided a method of treating locally advanced or metastatic urothelial carcinoma (mUC) comprising
(a) evaluating a biological sample from a subject in need of the treatment, in particular a patient with locally advanced or metastatic urothelial carcinoma, more in particular an adult patient with locally advanced or metastatic urothelial carcinoma, and wherein the subject, in particular the patient, more in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample. In certain embodiments, the locally advanced or metastatic UC is upper tract UC.

In an aspect, there is provided a method for the treatment of a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the subject, in particular the patient, more in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample. In certain embodiments, the locally advanced or metastatic UC is upper tract UC.

In an aspect, there is provided use of erdafitinib for the manufacture of a medicament for the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, received at least one line of systemic treatment containing an anti-PD-(L)1 agent in the locally advanced or metastatic treatment. In certain embodiments, the locally advanced or metastatic UC is upper tract UC.

In an aspect, there is provided use of erdafitinib for the manufacture of a medicament for the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, progressed on or after ≥1 prior treatment that included an anti-PD-(L)1 agent. In certain embodiments, the locally advanced or metastatic UC is upper tract UC. In an aspect, there is provided use of erdafitinib for the manufacture of a medicament for the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, progressed during or following at least one line of prior treatment with an anti-PD-(L)1 agent including within 12 months of neoadjuvant or adjuvant therapy. In certain embodiments, the locally advanced or metastatic UC is upper tract UC.

In an aspect, there is provided use of erdafitinib for the manufacture of a medicament for the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the locally advanced or metastatic urothelial carcinoma or the subject, in particular the patient, more in particular the adult patient, progressed during or following at least one line of prior treatment with an anti-PD-(L)1 agent in a neoadjuvant or adjuvant setting. In certain embodiments, the locally advanced or metastatic UC is upper tract UC.

In an aspect, there is provided use of erdafitinib for the manufacture of a medicament for the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who has received at least one line of systemic treatment containing an anti-PD-(L)1 agent in the locally advanced or metastatic treatment setting, wherein erdafitinib is administered or is to be administered in a therapeutically effective amount. In certain embodiments, the locally advanced or metastatic UC is upper tract UC.

In an aspect, there is provided use of erdafitinib for the manufacture of a medicament for the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who progressed on or after ≥1 prior treatment that included an anti-PD-(L)1 agent, wherein erdafitinib is administered or is to be administered in a therapeutically effective amount. In certain embodiments, the locally advanced or metastatic UC is upper tract UC.

In an aspect, there is provided use of erdafitinib for the manufacture of a medicament for the treatment of a subject in need thereof, in particular a patient with locally advanced or metastatic urothelial carcinoma, more in particular an adult patient with locally advanced or metastatic urothelial carcinoma, that has
one or more FGFR genetic alterations and
progressed during or following at least one line of prior treatment with an anti-PD-(L)1 agent including within 12 months of neoadjuvant or adjuvant therapy, wherein erdafitinib is administered or is to be administered in a therapeutically effective amount. In certain embodiments, the locally advanced or metastatic UC is upper tract UC.

In an aspect, there is provided use of erdafitinib for the manufacture of a medicament for the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the subject, in particular the patient, more in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample. In certain embodiments, the locally advanced or metastatic UC is upper tract UC.

In an aspect, there is provided use of erdafitinib for the manufacture of a medicament for the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the subject, in particular the patient, more in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample. In certain embodiments, the locally advanced or metastatic UC is upper tract UC.

In an aspect, there is provided erdafitinib for use in the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, received at least one line of systemic treatment containing an anti-PD-(L)1 agent in the locally advanced or metastatic treatment. In certain embodiments, the locally advanced or metastatic UC is upper tract UC.

In an aspect, there is provided erdafitinib for use in the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, progressed on or after ≥1 prior treatment that included an anti-PD-(L)1 agent. In certain embodiments, the locally advanced or metastatic UC is upper tract UC.

In an aspect, there is provided erdafitinib for use in the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, progressed during or following at least one line of prior treatment with an anti-PD-(L)1 agent including within 12 months of neoadjuvant or adjuvant therapy. In certain embodiments, the locally advanced or metastatic UC is upper tract UC.

In an aspect, there is provided erdafitinib for use in the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the locally advanced or metastatic urothelial carcinoma or the subject, in particular the patient, more in particular the adult patient, progressed during or following at least one line of prior treatment with an anti-PD-(L)1 agent in a neoadjuvant or adjuvant setting. In certain embodiments, the locally advanced or metastatic UC is upper tract UC.

In an aspect, there is provided erdafitinib for use in the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who received at least one line of systemic treatment containing an anti-PD-(L)1 agent in the locally advanced or metastatic treatment setting, wherein erdafitinib is administered or is to be administered in a therapeutically effective amount. In certain embodiments, the locally advanced or metastatic UC is upper tract UC.

In an aspect, there is provided erdafitinib for use in the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who progressed on or after ≥1 prior treatment that included an anti-PD-(L)1 agent, wherein erdafitinib is administered or is to be administered in a therapeutically effective amount. In certain embodiments, the locally advanced or metastatic UC is upper tract UC.

In an aspect, there is provided erdafitinib for use in the treatment of a subject in need thereof, in particular a patient with locally advanced or metastatic urothelial carcinoma, more in particular an adult patient with locally advanced or metastatic urothelial carcinoma that has
one or more FGFR genetic alterations and
progressed during or following at least one line of prior treatment with an anti-PD-(L)1 agent including within 12 months of neoadjuvant or adjuvant therapy, wherein erdafitinib is administered or is to be administered in a therapeutically effective amount. In certain embodiments, the locally advanced or metastatic UC is upper tract UC.

In an aspect, there is provided erdafitinib for use in the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the subject, in particular the patient, more in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample. In certain embodiments, the locally advanced or metastatic UC is upper tract UC.

In an aspect, there is provided erdafitinib for use in the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the subject, in particular the patient, more in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample. In certain embodiments, the locally advanced or metastatic UC is upper tract UC.

In an aspect there is provided a method of improving overall survival, in particular median overall survival, in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib.

In an aspect, there is provided a method of improving overall survival, in particular median overall survival, in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient a therapeutically effective amount of erdafitinib.

In an aspect, there is provided a method of improving overall survival, in particular median overall survival, in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient a therapeutically effective amount of erdafitinib.

In an aspect, there is provided a method of improving overall survival, in particular median overall survival, in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided a method of improving overall survival, in particular median overall survival, in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided use of erdafitinib for the manufacture of a medicament for improving overall survival, in particular median overall survival, in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.

In an aspect, there is provided use of erdafitinib for the manufacture of a medicament for improving overall survival, in particular median overall survival, in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.

In an aspect, there is provided use of erdafitinib for the manufacture of a medicament for improving overall survival, in particular median overall survival, in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.

In an aspect, there is provided use of erdafitinib for the manufacture of a medicament for improving overall survival, in particular median overall survival, in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided use of erdafitinib for the manufacture of a medicament for improving overall survival, in particular median overall survival, in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided erdafitinib for use in improving overall survival, in particular median overall survival, in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.

In an aspect, there is provided erdafitinib for use in improving overall survival, in particular median overall survival, in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.

In an aspect, there is provided erdafitinib for use in improving overall survival, in particular median overall survival, in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.

In an aspect, there is provided erdafitinib for use in improving overall survival, in particular median overall survival, in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided erdafitinib for use in improving overall survival, in particular median overall survival, in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect there is provided a method of reducing risk of death in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib.

In an aspect, there is provided a method of reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient a therapeutically effective amount of erdafitinib.

In an aspect, there is provided a method of reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient a therapeutically effective amount of erdafitinib.

In an aspect, there is provided a method of reducing risk of death in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided a method of reducing risk of death in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided use of erdafitinib for the manufacture of a medicament for reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.

In an aspect, there is provided use of erdafitinib for the manufacture of a medicament for reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.

In an aspect, there is provided use of erdafitinib for the manufacture of a medicament for reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.

In an aspect, there is provided use of erdafitinib for the manufacture of a medicament for reducing risk of death in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided use of erdafitinib for the manufacture of a medicament for reducing risk of death in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided erdafitinib for use in reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.

In an aspect, there is provided erdafitinib for use in reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.

In an aspect, there is provided erdafitinib for use in reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.

In an aspect, there is provided erdafitinib for use in reducing risk of death in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided erdafitinib for use in reducing risk of death in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect there is provided a method of improving median PFS in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib.

In an aspect, there is provided a method of improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient a therapeutically effective amount of erdafitinib.

In an aspect, there is provided a method of improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient a therapeutically effective amount of erdafitinib.

In an aspect, there is provided a method of improving median PFS in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided a method of improving median PFS in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided use of erdafitinib for the manufacture of a medicament for improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.

In an aspect, there is provided use of erdafitinib for the manufacture of a medicament for improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.

In an aspect, there is provided use of erdafitinib for the manufacture of a medicament for improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.

In an aspect, there is provided use of erdafitinib for the manufacture of a medicament for improving median PFS in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided use of erdafitinib for the manufacture of a medicament for improving median PFS in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided erdafitinib for use in improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.

In an aspect, there is provided erdafitinib for use in improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.

In an aspect, there is provided erdafitinib for use in improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.

In an aspect, there is provided erdafitinib for use in improving median PFS in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided erdafitinib for use in improving median PFS in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect there is provided a method of reducing risk of progression or death in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib.

In an aspect, there is provided a method of reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient a therapeutically effective amount of erdafitinib.

In an aspect, there is provided a method of reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient a therapeutically effective amount of erdafitinib.

In an aspect, there is provided a method of reducing risk of progression or death in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided a method of reducing risk of progression or death in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided use of erdafitinib for the manufacture of a medicament for reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.

In an aspect, there is provided use of erdafitinib for the manufacture of a medicament for reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.

In an aspect, there is provided use of erdafitinib for the manufacture of a medicament for reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.

In an aspect, there is provided use of erdafitinib for the manufacture of a medicament for reducing risk of progression or death in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided use of erdafitinib for the manufacture of a medicament for reducing risk of progression or death in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided erdafitinib for use in reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.

In an aspect, there is provided erdafitinib for use in reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.

In an aspect, there is provided erdafitinib for use in reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.

In an aspect, there is provided erdafitinib for use in reducing risk of progression or death in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided erdafitinib for use in reducing risk of progression or death in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect there is provided a method of improving ORR in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib. In certain embodiments, ORR is confirmed ORR by investigator assessment.

In an aspect, there is provided a method of improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient a therapeutically effective amount of erdafitinib. In certain embodiments, ORR is confirmed ORR by investigator assessment.

In an aspect, there is provided a method of improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient a therapeutically effective amount of erdafitinib. In certain embodiments, ORR is confirmed ORR by investigator assessment.

In an aspect, there is provided a method of improving ORR in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample. In certain embodiments, ORR is confirmed ORR by investigator assessment.

In an aspect, there is provided a method of improving ORR in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample. In certain embodiments, ORR is confirmed ORR by investigator assessment.

In an aspect, there is provided use of erdafitinib for the manufacture of a medicament for improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount. In certain embodiments, ORR is confirmed ORR by investigator assessment.

In an aspect, there is provided use of erdafitinib for the manufacture of a medicament for improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount. In certain embodiments, ORR is confirmed ORR by investigator assessment.

In an aspect, there is provided use of erdafitinib for the manufacture of a medicament for improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount. In certain embodiments, ORR is confirmed ORR by investigator assessment.

In an aspect, there is provided use of erdafitinib for the manufacture of a medicament for improving ORR in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample. In certain embodiments, ORR is confirmed ORR by investigator assessment.

In an aspect, there is provided use of erdafitinib for the manufacture of a medicament for improving ORR in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample. In certain embodiments, ORR is confirmed ORR by investigator assessment.

In an aspect, there is provided erdafitinib for use in improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount. In certain embodiments, ORR is confirmed ORR by investigator assessment.

In an aspect, there is provided erdafitinib for use in improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount. In certain embodiments, ORR is confirmed ORR by investigator assessment.

In an aspect, there is provided erdafitinib for use in improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount. In certain embodiments, ORR is confirmed ORR by investigator assessment.

In an aspect, there is provided erdafitinib for use in improving ORR in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample. In certain embodiments, ORR is confirmed ORR by investigator assessment.

In an aspect, there is provided erdafitinib for use in improving ORR in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample. In certain embodiments, ORR is confirmed ORR by investigator assessment.

In an aspect there is provided a method of improving disease control rate in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib.

In an aspect, there is provided a method of improving disease control rate in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient a therapeutically effective amount of erdafitinib.

In an aspect, there is provided a method of improving disease control rate in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient a therapeutically effective amount of erdafitinib.

In an aspect, there is provided a method of improving disease control rate in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided a method of improving disease control rate in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided use of erdafitinib for the manufacture of a medicament for improving disease control rate in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.

In an aspect, there is provided use of erdafitinib for the manufacture of a medicament for improving disease control rate in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.

In an aspect, there is provided use of erdafitinib for the manufacture of a medicament for improving disease control rate in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.

In an aspect, there is provided use of erdafitinib for the manufacture of a medicament for improving disease control rate in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided use of erdafitinib for the manufacture of a medicament for improving disease control rate in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided erdafitinib for use in improving disease control rate in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.

In an aspect, there is provided erdafitinib for use in improving disease control rate in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.

In an aspect, there is provided erdafitinib for use in improving disease control rate in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.

In an aspect, there is provided erdafitinib for use in improving disease control rate in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In an aspect, there is provided erdafitinib for use in improving disease control rate in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.

In any of the foregoing embodiments, the improvement may be relative to a reference population of patients with locally advanced or metastatic urothelial carcinoma, in particular a reference population of adult patients with locally advanced or metastatic urothelial carcinoma, said reference population having been administered chemotherapy. In certain embodiments, the chemotherapy is docetaxel administered at a dose of 75 mg per square meter once every 3 weeks, administered intravenously over 1 hour. In certain embodiments, the chemotherapy is vinflunine administered at a dose of 320 mg per square meter once every 3 weeks, administered intravenously over 20 minutes.

In an aspect there is provided a method of achieving a favorable tolerability profile in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib, wherein the favorable tolerability profile is relative to the tolerability profile of chemotherapy, in particular single-agent chemotherapy.

In an aspect, there is provided a method of achieving a favorable tolerability profile in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient a therapeutically effective amount of erdafitinib, wherein the favorable tolerability profile is relative to the tolerability profile of chemotherapy, in particular single-agent chemotherapy.

In an aspect, there is provided a method of achieving a favorable tolerability profile in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient a therapeutically effective amount of erdafitinib, wherein the favorable tolerability profile is relative to the tolerability profile of chemotherapy, in particular single-agent chemotherapy.

In an aspect, there is provided a method of achieving a favorable tolerability profile in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample, wherein the favorable tolerability profile is relative to the tolerability profile of chemotherapy, in particular single-agent chemotherapy.

In an aspect, there is provided a method of achieving a favorable tolerability profile in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample, wherein the favorable tolerability profile is relative to the tolerability profile of chemotherapy, in particular single-agent chemotherapy.

In an aspect, there is provided use of erdafitinib for the manufacture of a medicament for achieving a favorable tolerability profile in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount, wherein the favorable tolerability profile is relative to the tolerability profile of chemotherapy, in particular single-agent chemotherapy.

In an aspect, there is provided use of erdafitinib for the manufacture of a medicament for achieving a favorable tolerability profile in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount, wherein the favorable tolerability profile is relative to the tolerability profile of chemotherapy, in particular single-agent chemotherapy.

In an aspect, there is provided use of erdafitinib for the manufacture of a medicament for achieving a favorable tolerability profile in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount, wherein the favorable tolerability profile is relative to the tolerability profile of chemotherapy, in particular single-agent chemotherapy.

In an aspect, there is provided use of erdafitinib for the manufacture of a medicament for achieving a favorable tolerability profile in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample, wherein the favorable tolerability profile is relative to the tolerability profile of chemotherapy, in particular single-agent chemotherapy.

In an aspect, there is provided use of erdafitinib for the manufacture of a medicament for achieving a favorable tolerability profile in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample, wherein the favorable tolerability profile is relative to the tolerability profile of chemotherapy, in particular single-agent chemotherapy.

In an aspect, there is provided erdafitinib for use in achieving a favorable tolerability profile in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount, wherein the favorable tolerability profile is relative to the tolerability profile of chemotherapy, in particular single-agent chemotherapy.

In an aspect, there is provided erdafitinib for use in achieving a favorable tolerability profile in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount, wherein the favorable tolerability profile is relative to the tolerability profile of chemotherapy, in particular single-agent chemotherapy.

In an aspect, there is provided erdafitinib for use in achieving a favorable tolerability profile in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount, wherein the favorable tolerability profile is relative to the tolerability profile of chemotherapy, in particular single-agent chemotherapy.

In an aspect, there is provided erdafitinib for use in achieving a favorable tolerability profile in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample, wherein the favorable tolerability profile is relative to the tolerability profile of chemotherapy, in particular single-agent chemotherapy.

In an aspect, there is provided erdafitinib for use in achieving a favorable tolerability profile in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample, wherein the favorable tolerability profile is relative to the tolerability profile of chemotherapy, in particular single-agent chemotherapy.

In any of the embodiments disclosed herein in which an improvement or reduction is mentioned, the improvement or reduction may be relative to a reference patient or a reference population of patients with locally advanced or metastatic urothelial carcinoma, in particular a reference adult patient or population of adult patients with locally advanced or metastatic urothelial carcinoma, said reference patient or population having been administered chemotherapy instead of erdafitinib. In certain embodiments, the chemotherapy is docetaxel or vinflunine.

“Reference patient” or “reference population of patients” refers to a subject or population of subjects having locally advanced or metastatic urothelial carcinoma, who have been administered chemotherapy such as for example docetaxel or vinflunine, instead of erdafitinib. The reference subject or reference population of subjects are substantially the same as the subject or population of subjects prior to treatment with chemotherapy or erdafitinib, respectively. Exemplary reference subjects or reference population of subjects are those described in Example 1 or Example 2 herein. In some embodiments, the population of subjects and reference population of subjects contain at least two subjects. In some embodiments, the population of subjects and reference population of subjects contain a number of subjects that allow for a statistically significant analysis of the improvement in safety and/or efficacy.

In any of the embodiments disclosed herein in which progression is mentioned, it is to be understood that reference to progression of a patient or an adult patient is interchangeable with progression of the cancer of the patient or adult patient. For example, the phrase “the patient, in particular the adult patient, progressed during or following at least one line of prior treatment with an anti-PD-(L)1 agent” is interchangeable with the phrase “whose diseaseprogressed during or following at least one line of prior treatment with an anti-PD-(L)1 agent”, in particular “whose urothelial carcinoma progressed during or following at least one line of prior treatment with an anti-PD-(L)1 agent”, more in particular “whose locally advanced or metastatic urothelial carcinoma progressed during or following at least one line of prior treatment with an anti-PD-(L)1 agent”.

In any of the foregoing embodiments, wherein it is stated that the locally advanced or metastatic urothelial carcinoma or the patient or the population of patients, in particular the adult patient or the population of adult patients, progressed during or following at least one line of prior treatment with an anti-PD-(L)1 agent including in a neoadjuvant or adjuvant setting, it is to be understood that the disease state prior to progression includes, for example non-metastatic disease such as non-metastatic urothelial carcinoma.

### BRIEF DESCRIPTION OF THE FIGURES

The invention will be described, by way of example only, with reference to the accompanying figures.

FIG. 1: study design of phase 3 study NCT03390504 (Study 42756493-BLC3001). Cohort 1 had a planned enrollment of 280 subjects.

FIG. 2: Kaplan-Meier Plot of Overall Survival; Cohort 1 ITT Analysis Set (Study 42756493-BLC3001)

FIG. 3: Kaplan-Meier Plot of Progression-free Survival; Cohort 1 ITT Analysis Set (Study 42756493-BLC3001)

FIG. 4: Objective Response Rate for erdafitinib treatment and chemotherapy (Study 42756493-BLC3001).

FIG. 5A: is a CONSORT diagram showing THOR (Study 42756493-BLC3001) screening patient flow. FIG. 5B shows THOR Study Design. A red box is drawn around THOR cohort 1. FIG. 5C is a CONSORT diagram showing THOR Cohort 1 patient flow. Three patients who did not receive a prior anti-PD-(L)1 agent were incorrectly assigned to Cohort 1 (erdafitinib, n=1; chemotherapy, n=2). Due to global shortage of vinflunine during the study, from June to December 2022, new patients assigned to the chemotherapy group could only receive docetaxel. Patients who received treatment with vinflunine in the study continued to receive vinflunine. Paclitaxel was not included in the investigator choice of chemotherapy options, as at the time this study was designed, docetaxel and vinflunine were the most commonly prescribed chemotherapy agents in participating countries. FIGS. 5D and 5E are graphs showing baseline FGFR alterations. Footnotes for FIGS. 5D and 5E: *Intent-to-treat population (two patients were false positive for FGFR alterations [erdafitinib group, n=1; chemotherapy group, n=1] ^{†}Patients with FGFR mutations only; ^{‡}Patients with FGFR fusions only. ^{§} FGFR mutations and fusions (erdafitinib group, n=2; chemotherapy group, n=3) are delineated in Table 17.

FIGS. 6A and 6B show overall survival. FIG. 6A is a Kaplan-Meier estimate of overall survival by treatment group. FIG. 6B is a diagram showing overall survival according to key subgroups. The vertical dotted line represents the hazard ration (HR) for the overall population for comparison purposes. CI, confidence interval; CPS, combined positive score; ECOG PS, Eastern Cooperative Oncology Group performance status; PD-1, programmed cell death protein 1; PD-L1, programmed death-ligand 1.

FIGS. 7A and 7B are graphs showing key secondary end points. FIG. 7A is a Kaplan-Meier estimate of progression-free survival by treatment group. FIG. 7B is a graph showing Progression-Free Survival. FIG. 7C is a graph showing objective response rate according to key subgroups. CI, confidence interval; CPS, combined positive score; CR, complete response; ECOG PS, Eastern Cooperative Oncology Group performance status; PD-1, programmed cell death protein 1; PD-L1, programmed death-ligand 1; PR, partial response.

FIG. 8 shows a Forest Plot of Overall Survival—Subgroup Analysis; Cohort 1 ITT Analysis Set (Study 42756493-BLC3001)

FIG. 9 shows Forest Plot of Overall Survival—Subgroup Analysis; Cohort 1 ITT Analysis Set (Study 42756493-BLC3001)

FIG. 10 shows a Kaplan-Meier Plot of Overall Survival (Study BLC3001 Cohort 1)

FIG. 11 shows a Kaplan-Meier Plot of Progression-free Survival (Study BLC3001 Cohort 1)

### DETAILED DESCRIPTION OF THE INVENTION

Erdafitinib or N-(3,5-dimethoxyphenyl)-N′-(1-methylethyl)-N-[3-(1-methyl-1H-pyrazol-4-yl)quinoxalin-6-yl]ethane-1,2-diamine is a pan-fibroblast growth factor receptor (FGFR 1,2,3,4) tyrosine kinase inhibitor.

The chemical structure of erdafitinib is

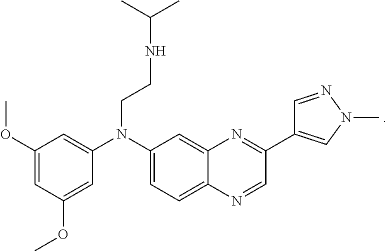

In an embodiment, the cancer as described herein is locally advanced or metastatic urothelial carcinoma. In an embodiment, the cancer as described herein is upper tract urothelial carcinoma.

In an embodiment the locally advanced or metastatic urothelial carcinoma mentioned throughout the specification is locally advanced unresectable or metastatic urothelial carcinoma (mUC).

In an embodiment, in the aspects of the invention, the one or more FGFR genetic alterations are FGFR fusions and/or mutations, in particular FGFR fusions and/or mutations sensitive to erdafitinib. In an embodiment, the FGFR fusions and/or mutations are FGFR3 mutations and/or FGFR2 and 3 fusions. In an embodiment, the one or more FGFR genetic alterations are selected from FGFR3-TACC3, in particular FGFR3-TACC3 v1 and FGFR3-TACC3 v3; FGFR3-BAIAP2L1; FGFR2-BICC1; FGFR2-CASP7; FGFR3 R248C; FGFR3 S249C; FGFR3 G370C; and FGFR3 Y373C. In an embodiment, the one or more FGFR genetic alterations are FGFR3 mutations and/or FGFR3 fusions. In an embodiment, the one or more FGFR genetic alterations are selected from FGFR3-TACC3, in particular FGFR3-TACC3 v1 and FGFR3-TACC3 v3; FGFR3-BAIAP2L1; FGFR3 R248C; FGFR3 S249C; FGFR3 G370C; and FGFR3 Y373C.

In an embodiment, the one or more FGFR genetic alterations is FGFR3 S249C.

In an embodiment, the one or more FGFR genetic alterations is FGFR3 Y373C.

In an embodiment, the one or more FGFR genetic alterations is FGFR3 G370C.

In an embodiment, the one or more FGFR genetic alterations is FGFR3 R248C.

In an embodiment, the one or more FGFR genetic alterations is FGFR3-TACC3 v1.

In an embodiment, the one or more FGFR genetic alterations is FGFR3-TACC3 v3.

In an embodiment, the one or more FGFR genetic alterations is FGFR3-BAIAP2L1.

In an embodiment, the one or more FGFR genetic alterations is FGFR2-BICC1.

In an embodiment, the one or more FGFR genetic alterations is FGFR2-CASP7.

In an embodiment, the one or more FGFR genetic alterations are FGFR3 R248C and FGFR3 Y373C.

In an embodiment, the one or more FGFR genetic alterations are FGFR3 S249C and FGFR3 Y373C.

In an embodiment, the one or more FGFR genetic alterations are FGFR3 G370C and FGFR3 Y373C.

In an embodiment, the one or more FGFR genetic alterations are FGFR3 R248C and FGFR3 S249C.

In an embodiment, the one or more FGFR genetic alterations are FGFR3 G370C and FGFR3 S249C and FGFR3 Y373C.

In an embodiment, the one or more FGFR genetic alterations are FGFR3 R248C and FGFR3 S249C and FGFR3 Y373C.

In an embodiment, the one or more FGFR genetic alterations are FGFR3-BAIAP2L1 and FGFR3-TACC3_V1.

In an embodiment, the one or more FGFR genetic alterations are FGFR3-TACC3_V1 and FGFR3-TACC3_V3.

In an embodiment, the one or more FGFR genetic alterations are FGFR3 S249C and FGFR3-TACC3_V1.

In an embodiment, the one or more FGFR genetic alterations are FGFR3 S249C and FGFR3-TACC3 V3.

In an embodiment, the one or more FGFR genetic alterations are FGFR3 Y373C and FGFR3-TACC3_V1.

In an embodiment, the one or more FGFR genetic alterations are FGFR3 R248C and FGFR3-TACC3_V1.

In an embodiment, the cancer as described herein is locally advanced or metastatic urothelial carcinoma harboring FGFR3 S249C.

In an embodiment, the cancer as described herein is locally advanced or metastatic urothelial carcinoma harboring FGFR3 Y373C.

In an embodiment, the cancer as described herein is locally advanced or metastatic urothelial carcinoma harboring FGFR3 G370C.

In an embodiment, the cancer as described herein is locally advanced or metastatic urothelial carcinoma harboring FGFR3 R248C.

In an embodiment, the cancer as described herein is locally advanced or metastatic urothelial carcinoma harboring FGFR3-TACC3 v1.

In an embodiment, the cancer as described herein is locally advanced or metastatic urothelial carcinoma harboring FGFR3-TACC3 v3.

In an embodiment, the cancer as described herein is locally advanced or metastatic urothelial carcinoma harboring FGFR3-BAIAP2L1.

In an embodiment, the cancer as described herein is locally advanced or metastatic urothelial carcinoma harboring FGFR2-BICC1.

In an embodiment, the cancer as described herein is locally advanced or metastatic urothelial carcinoma harboring FGFR2-CASP7.

In an embodiment, the cancer as described herein is locally advanced or metastatic urothelial carcinoma harboring FGFR3 R248C and FGFR3 Y373C.

In an embodiment, the cancer as described herein is locally advanced or metastatic urothelial carcinoma harboring FGFR3 S249C and FGFR3 Y373C.

In an embodiment, the cancer as described herein is locally advanced or metastatic urothelial carcinoma harboring FGFR3 G370C and FGFR3 Y373C.

In an embodiment, the cancer as described herein is locally advanced or metastatic urothelial carcinoma harboring FGFR3 R248C and FGFR3 S249C.

In an embodiment, the cancer as described herein is locally advanced or metastatic urothelial carcinoma harboring FGFR3 G370C and FGFR3 S249C and FGFR3 Y373C.

In an embodiment, the cancer as described herein is locally advanced or metastatic urothelial carcinoma harboring FGFR3 R248C and FGFR3 S249C and FGFR3 Y373C.

In an embodiment, the cancer as described herein is locally advanced or metastatic urothelial carcinoma harboring FGFR3-BAIAP2L1 and FGFR3-TACC3_V1.

In an embodiment, the cancer as described herein is locally advanced or metastatic urothelial carcinoma harboring FGFR3-TACC3_V1 and FGFR3-TACC3_V3.

In an embodiment, the cancer as described herein is locally advanced or metastatic urothelial carcinoma harboring FGFR3 S249C and FGFR3-TACC3_V1.

In an embodiment, the cancer as described herein is locally advanced or metastatic urothelial carcinoma harboring FGFR3 S249C and FGFR3-TACC3_V3.

In an embodiment, the cancer as described herein is locally advanced or metastatic urothelial carcinoma harboring FGFR3 Y373C and FGFR3-TACC3_V1.

In an embodiment, the cancer as described herein is locally advanced or metastatic urothelial carcinoma harboring FGFR3 R248C and FGFR3-TACC3_V1.

In an embodiment, in the aspects of the invention, the patient with locally advanced or metastatic urothelial carcinoma (mUC) is positive for PD-(L)1 expression. In an embodiment, the patient with locally advanced or metastatic urothelial carcinoma (mUC) is harboring one or more fibroblast growth factor receptor (FGFR) genetic alterations, in particular one or more FGFR3 genetic alterations, and is positive for PD-(L)1 expression.

In certain embodiments, the PD-(L)1 expression is low, in particular the PD-(L)1 expression has a combined positive score of <10 on a PD-L1 immunohistochemical 22C3 assay.

In an embodiment, in the aspects of the invention, the patient with locally advanced or metastatic urothelial carcinoma (mUC) is negative for PD-(L)1 expression. In an embodiment, the patient with locally advanced or metastatic urothelial carcinoma (mUC) is harboring one or more fibroblast growth factor receptor (FGFR) genetic alterations, in particular one or more FGFR3 genetic alterations, and is negative for PD-(L)1 expression.

In an embodiment, in the aspects of the invention, the method, use or for use as described herein, provides for an improvement of OS, in particular median OS, more in particular OS is significantly prolonged versus chemotherapy. In an embodiment, the method, use or for use as described herein provides for a median OS of about 12.1 months. In an embodiment, the method, use or for use as described herein provides for a median OS of about 12.1 months versus a median OS of about 7.8 months for chemotherapy (HR=0.64; 95% CI. 0.47, 0.88; p-value=0.0050). In an embodiment, the chemotherapy is docetaxel or vinflunine, in particular docetaxel 75 mg/m2 once every 3 weeks or vinflunine 320 mg/m2 once every 3 weeks.

In an embodiment, in the aspects of the invention, the method, use or for use as described herein, provides for a reduced risk of death, in particular a reduced risk of death versus chemotherapy. In an embodiment, the method, use or for use as described herein reduced the risk of death by about 36% versus chemotherapy. In an embodiment, the chemotherapy is docetaxel or vinflunine, in particular docetaxel 75 mg/m2 once every 3 weeks or vinflunine 320 mg/m2 once every 3 weeks.

In an embodiment, in the aspects of the invention, the method, use or for use as described herein, provides for an improvement of median PFS, in particular PFS is significantly prolonged versus chemotherapy. In an embodiment, the method, use or for use as described herein provides for a median PFS of about 5.6 months. In an embodiment, the method, use or for use as described herein provides for a median PFS of about 5.6 months versus a median PFS of about 2.7 months for chemotherapy (HR, 0.58 (95% CI, 0.44-0.78; p=0.0002). In an embodiment, the chemotherapy is docetaxel or vinflunine, in particular docetaxel 75 mg/m2 once every 3 weeks or vinflunine 320 mg/m2 once every 3 weeks.

In an embodiment, in the aspects of the invention, the method, use or for use as described herein, provides for a reduced risk of progression or death, in particular a reduced risk of progression or death versus chemotherapy. In an embodiment, the method, use or for use as described herein reduced the risk of progression or death by about 42% versus chemotherapy. In an embodiment, the chemotherapy is docetaxel or vinflunine, in particular docetaxel 75 mg/m2 once every 3 weeks or vinflunine 320 mg/m2 once every 3 weeks.

In an embodiment, in the aspects of the invention, the method, use or for use as described herein, provides for an improvement of ORR, in particular ORR is significantly higher versus chemotherapy. In an embodiment, the method, use or for use as described herein provides for an ORR (CR+PR) of about 45.6% (CR 6.6%; PR 39.0%), in particular as determined by investigator assessment. In an embodiment, the method, use or for use as described herein provides for an ORR (CR+PR) of about 45.6% versus an ORR (CR+PR) of about 11.5% (CR 0.8%; PR 10.8%) for chemotherapy, in particular as determined by investigator assessment (Relative Risk, 3.94 (95% CI, 2.37-6.57), p<0.001). In an embodiment, the method, use or for use as described herein provides for a confirmed ORR (CR+PR; ≥2 consecutive assessments) of about 35.3% (CR 5.1%; PR 30.1%), in particular as determined by investigator assessment. In an embodiment, the method, use or for use as described herein provides for a confirmed ORR (CR+PR; ≥2 consecutive assessments) of about 35.3% versus a confirmed ORR (CR+PR) of about 8.5% (CR 0.8%; PR 7.7%) for chemotherapy, in particular as determined by investigator assessment (Relative Risk, 3.94 (95% CI, 2.37-6.57), p<0.001). In an embodiment, the chemotherapy is docetaxel or vinflunine, in particular docetaxel 75 mg/m2 once every 3 weeks or vinflunine 320 mg/m2 once every 3 weeks. In an embodiment the ORR is by investigator assessment. In an embodiment the ORR is confirmed by investigator assessment. In an embodiment, the method, use or for use as described herein provides for a confirmed objective response rate by investigator assessment of about 35.3% versus of about 8.5% for chemotherapy.

In an embodiment, in the aspects of the invention, the method, use or for use as described herein, provides for an improved disease control rate, in particular an improved disease control rate versus chemotherapy. In an embodiment, the method, use or for use as described herein improves disease control rate by about 39% versus chemotherapy. In an embodiment, the method, use or for use as described herein provides for a disease control rate of about 82.4%. Chemotherapy provides for a disease control rate of about 43.1%. In an embodiment, the chemotherapy is docetaxel or vinflunine, in particular docetaxel 75 mg/m2 once every 3 weeks or vinflunine 320 mg/m2 once every 3 weeks.

In an embodiment, in the aspects of the invention, the method, use or for use as described herein, provides for a favorable tolerability profile, in particular a favorable tolerability profile rate versus chemotherapy, in particular single-line chemotherapy. In an embodiment, the chemotherapy is docetaxel or vinflunine, in particular docetaxel 75 mg/m2 once every 3 weeks or vinflunine 320 mg/m2 once every 3 weeks.

In an embodiment, the method, use or for use as described herein provides for a median DOR of about 4.9 months. Chemotherapy provides for a median DOR of about 5.6 months.

In an embodiment, in the aspects of the invention, the method, use or for use as described herein, provides for a lower discontinuation due to treatment-related adverse events, in particular a lower discontinuation due to treatment-related adverse events versus chemotherapy. In an embodiment, the method, use or for use as described herein lowers discontinuation due to treatment-related adverse events by about 5.3% versus chemotherapy. In an embodiment, the method, use or for use as described herein provides for a discontinuation due to treatment-related adverse events of about 8.1%. Chemotherapy provides for a discontinuation due to treatment-related adverse events of about 13.4%. In an embodiment, the chemotherapy is docetaxel or vinflunine, in particular docetaxel 75 mg/m2 once every 3 weeks or vinflunine 320 mg/m2 once every 3 weeks.

In an embodiment, in the aspects of the invention, the therapeutically effective amount of erdafitinib is 8 mg daily, in particular 8 mg once daily.

In an embodiment, in the aspects of the invention, the therapeutically effective amount of erdafitinib is 9 mg daily, in particular 9 mg once daily.

In an embodiment, in the aspects of the invention, the starting dose of erdafitinib is 8 mg daily, in particular once daily, with potential up-titration to 9 mg daily, in particular once daily, based on observed serum phosphate level, in particular serum phosphate level assessed on any of day 14 to 21 after initiating erdafitinib treatment. In an embodiment, to determine whether or not to up-titrate the dose of erdafitinib from 8 mg once daily to 9 mg once daily, serum phosphate level is assessed on day 14 of erdafitinib treatment. In an embodiment, to determine whether or not to up-titrate the dose of erdafitinib from 8 mg once daily to 9 mg once daily, serum phosphate level is assessed on day 15 of erdafitinib treatment. In an embodiment, to determine whether or not to up-titrate the dose of erdafitinib from 8 mg once daily to 9 mg once daily, serum phosphate level is assessed on day 16 of erdafitinib treatment. In an embodiment, to determine whether or not to up-titrate the dose of erdafitinib from 8 mg once daily to 9 mg once daily, serum phosphate level is assessed on day 17 of erdafitinib treatment. In an embodiment, to determine whether or not to up-titrate the dose of erdafitinib from 8 mg once daily to 9 mg once daily, serum phosphate level is assessed on day 18 of erdafitinib treatment. In an embodiment, to determine whether or not to up-titrate the dose of erdafitinib from 8 mg once daily to 9 mg once daily, serum phosphate level is assessed on day 19 of erdafitinib treatment. In an embodiment, to determine whether or not to up-titrate the dose of erdafitinib from 8 mg once daily to 9 mg once daily, serum phosphate level is assessed on day 20 of erdafitinib treatment. In an embodiment, to determine whether or not to up-titrate the dose of erdafitinib from 8 mg once daily to 9 mg once daily, serum phosphate level is assessed on day 21 of erdafitinib treatment. In an embodiment, to determine whether or not to up-titrate the dose of erdafitinib from 8 mg once daily to 9 mg once daily, serum phosphate level is assessed between 14 and 21 days after initiating erdafitinib treatment. In an embodiment, to determine whether or not to up-titrate the dose of erdafitinib from 8 mg once daily to 9 mg once daily, serum phosphate level is assessed on any one of days 14 to and including 21 after initiating erdafitinib treatment. In an embodiment, erdafitinib dose is uptitrated to 9 mg daily, in particular once daily, if serum phosphate level is <5.5 mg/dL. In an embodiment, erdafitinib dose is uptitrated to 9 mg daily, in particular once daily, if serum phosphate level is <7 mg/dL. In an embodiment, erdafitinib dose is uptitrated to 9 mg daily, in particular once daily, if serum phosphate level is <9 mg/dL. In an embodiment, erdafitinib dose is uptitrated to 9 mg daily, in particular once daily, if serum phosphate level is ranging from and including 7.00 to 8.99 mg/dL while concurrently initiating treatment with a phosphate binder such as for example sevelamer.

In an embodiment, in the aspects of the invention, the starting dose of erdafitinib is 8 mg daily, in particular once daily, with up-titration to 9 mg daily, in particular once daily, if the serum phosphate level assessed on any one of days 14 to and including 21 of initiating erdafitinib treatment is <5.5 mg/dL.

In an embodiment, in the aspects of the invention, the starting dose of erdafitinib is 8 mg daily, in particular once daily, with up-titration to 9 mg daily, in particular once daily, if the serum phosphate level assessed on any one of days 14 to and including 21 of initiating erdafitinib treatment is <5.5 mg/dL and there is no drug-related toxicity.

In an embodiment, in the aspects of the invention, the starting dose of erdafitinib is 8 mg daily, in particular once daily, with up-titration to 9 mg daily, in particular once daily, if the serum phosphate level assessed on any one of days 14 to and including 21 of initiating erdafitinib treatment is <5.5 mg/dL and there are no ocular disorders or Grade 2 or greater adverse reactions.

In an embodiment, in the aspects of the invention, the starting dose of erdafitinib is 8 mg daily, in particular once daily, with up-titration to 9 mg daily, in particular once daily, if the serum phosphate level assessed on any one of days 14 to and including 21 of initiating erdafitinib treatment is <7.0 mg/dL.

In an embodiment, in the aspects of the invention, the starting dose of erdafitinib is 8 mg daily, in particular once daily, with up-titration to 9 mg daily, in particular once daily, if the serum phosphate level assessed on any one of days 14 to and including 21 of initiating erdafitinib treatment is <7.0 mg/dL and there is no drug-related toxicity.

In an embodiment, in the aspects of the invention, the starting dose of erdafitinib is 8 mg daily, in particular once daily, with up-titration to 9 mg daily, in particular once daily, if the serum phosphate level assessed on any one of days 14 to and including 21 of initiating erdafitinib treatment is <7.0 mg/dL and there are no ocular disorders or Grade 2 or greater adverse reactions.

In an embodiment, in the aspects of the invention, the starting dose of erdafitinib is 8 mg daily, in particular once daily, with up-titration to 9 mg daily, in particular once daily, if the serum phosphate level assessed on any one of days 14 to and including 21 of initiating erdafitinib treatment is <9.0 mg/dL. In an embodiment, the starting dose of erdafitinib is 8 mg daily, in particular once daily, with up-titration to 9 mg daily, in particular once daily, if the serum phosphate level assessed on any one of days 14 to and including 21 of initiating erdafitinib treatment is ranging from and including 7.00 to 8.99 mg/dL while concurrently initiating treatment with a phosphate binder such as for example sevelamer.

In an embodiment, in the aspects of the invention, the starting dose of erdafitinib is 8 mg daily, in particular once daily, with up-titration to 9 mg daily, in particular once daily, if the serum phosphate level assessed on any one of days 14 to and including 21 of initiating erdafitinib treatment is <9.0 mg/dL and there is no drug-related toxicity. In an embodiment, the starting dose of erdafitinib is 8 mg daily, in particular once daily, with up-titration to 9 mg daily, in particular once daily, if the serum phosphate level assessed on any one of days 14 to and including 21 of initiating erdafitinib treatment is ranging from and including 7.00 to 8.99 mg/dL while concurrently initiating treatment with a phosphate binder such as for example sevelamer and there is no drug-related toxicity.

In an embodiment, in the aspects of the invention, the starting dose of erdafitinib is 8 mg daily, in particular once daily, with up-titration to 9 mg daily, in particular once daily, if the serum phosphate level assessed on any one of days 14 to and including 21 of initiating erdafitinib treatment is <9.0 mg/dL and there are no ocular disorders or Grade 2 or greater adverse reactions. In an embodiment, the starting dose of erdafitinib is 8 mg daily, in particular once daily, with up-titration to 9 mg daily, in particular once daily, if the serum phosphate level assessed on any one of days 14 to and including 21 of initiating erdafitinib treatment is ranging from and including 7.00 to 8.99 mg/dL while concurrently initiating treatment with a phosphate binder such as for example sevelamer and there are no ocular disorders or Grade 2 or greater adverse reactions.

In an embodiment, the 8 mg dose of erdafitinib is administered or is to be administered as 2 formulations, in particular 2 tablets, each comprising 4 mg of erdafitinib. In an embodiment, the 8 mg dose of erdafitinib is administered or is to be administered as 2 formulations, in particular 2 tablets, one comprising 3 mg of erdafitinib and one comprising 5 mg of erdafitinib. In a preferred embodiment the 8 mg dose of erdafitinib is administered or is to be administered as 2 formulations, in particular 2 tablets, each comprising 4 mg of erdafitinib.

In an embodiment, the 9 mg dose of erdafitinib can be administered as 3 formulations, in particular 3 tablets, each comprising 3 mg of erdafitinib. In an embodiment, the 9 mg dose of erdafitinib is administered or is to be administered as 2 formulations, in particular 2 tablets, one comprising 4 mg of erdafitinib and one comprising 5 mg of erdafitinib. In a preferred embodiment the 9 mg dose of erdafitinib is administered or is to be administered as 3 formulations, in particular 3 tablets, each comprising 3 mg of erdafitinib.

It is to be understood that when the serum phosphate level is measured as a pharmacodynamic marker for determining whether or not to up-titrate the 8 mg starting dose of erdafitinib, in particular measured on any one of day 14 to 21 of erdafitinib administration, in particular on any one of day 14±2 days, of erdafitinib administration, phosphate levels may be further monitored during erdafitinib treatment. In an embodiment, clinical management of serum phosphate levels is done as represented in Table 5A.

TABLE 5A
Guidelines for Management of Serum Phosphate Elevation

| Serum Phosphate | | |
|---|---|---|
| Level | Study Drug Management | Symptom Management |
| <5.50 mg/dL | Continue erdafitinib treatment. | None. |
| (<1.75 mmol/L) | | |
| (Grade 0) | | |
| 5.50-6.99 mg/dL | Continue erdafitinib treatment. | Restriction of phosphate |
| (1.75-2.24 | | intake to 600-800 |
| mmol/L) (Grade | | mg/day. |
| 1) | | |
| 7.00-8.99 mg/dL | Continue erdafitinib treatment. | Restriction of phosphate |
| (2.25-2.90 | A dose reduction will be implemented | intake |
| mmol/L) (Grade | for persistent^{a} hyperphosphatemia | to 600-800 mg/day. |
| 2) | (defined as serum phosphate ≥7.00 | Phosphate binders (eg, |
| | mg/dL for a period of 2 months) or if | sevelamer 800 to 1,600 mg |
| | clinically necessary (eg, in the | TID with food) until |
| | presence of additional adverse events | phosphate level is <7.00 |
| | linked to | mg/dL. (See information |
| | hyperphosphatemia or electrolyte | regarding phosphate |
| | disturbances) | binders other than |
| | | sevelamer in the table |
| | | footnote.) |
| 9.00-10.00 | Withhold^{b} erdafitinib treatment until | Restriction of phosphate |
| mg/dL (2.91-3.20 | serum phosphate level returns to <7.00 | intake to 600-800 |
| mmol/L) (Grade | mg/dL (weekly testing recommended). | mg/day. |
| 3) | Re-start treatment at the same dose | Phosphate binders (eg, |
| | level. | sevelamer up to 1,600 mg |
| | A dose reduction will be implemented | TID with food) until |
| | for persistent^{a} hyperphosphatemia | serum phosphate level is |
| | (defined as serum phosphate ≥9.00 | <7.00 mg/dL. (See |
| | mg/dL for a period of 1 month) or if | information regarding |
| | clinically necessary (eg, in the | phosphate binders other |
| | presence of additional adverse events | than sevelamer in the table |
| | linked to hyperphosphatemia or | footnote.) |
| | electrolyte disturbances) | |
| >10.00 mg/dL | Withhold^{b} erdafitinib treatment until | Medical management as |
| (>3.20 mmol/L) | serum phosphate level returns to 7.00 | clinically appropriate. |
| (Grade 4) | mg/dL (weekly testing recommended). | |
| | Re-start treatment at the first reduced | |
| | dose level. If persistent^{a} | |
| | hyperphosphatemia (≥10.00 mg/dL) | |
| | for >2 weeks, | |
| | erdafitinib should be discontinued | |
| | permanently | |
| Significant | Erdafitinib should be discontinued | Medical management as |
| alteration in | permanently. (In situations where the | clinically appropriate. |
| baseline renal | subject is having clinical benefit and | |
| function or Grade | the investigator and the sponsor's | |
| 3 hypocalcemia | medical monitor agree that | |
| | continuation of treatment is in the best | |
| | interest of the subject, the drug may be | |
| | re-started at 2 dose levels lower if | |
| | appropriate. | |

TID = 3 times a day.
Note:
These are general guidelines that are based on emerging data and consensus experience of participating investigators or the experts in the field. The treating physicians must use clinical judgment and local standard of care to decide the best way to manage phosphate elevation. If sevelamer hydrochloride (Renagel ®) is not available, use of other phosphate binders (non- calcium containing) based on the local standard is recommended, including sevelamer carbonate (Renvela) or lanthanum carbonate (Fosrenol ®). These guidelines will be updated based on emerging data.
^{a}Persistent hyperphosphatemia is considered to be more than 1 sequential phosphate value above the cut-off.
^{b}Study drug interruptions for hyperphosphatemia suggested to be 7 days in duration.

The recommended dose modifications for adverse reactions including hyperphosphatemia are listed in Table 6A.

TABLE 6A
Erdafitinib dose reduction schedule

| | | 1^{st} | 2^{nd} | 3^{rd} | 4^{th} | 5^{th} |
|---|---|---|---|---|---|---|
| | | dose | dose | dose | dose | dose |
| Dose | | reduction | reduction | reduction | reduction | reduction |
| 9 mg | [symbol] | 8 mg | 6 mg | 5 mg | 4 mg | Stop |
| (three 3 | | (two 4 mg | (two 3 mg | (one 5 mg | (one 4 mg | |
| mg | | tablets) | tablets) | tablet) | tablet) | |
| tablets) | | | | | | |
| 8 mg | [symbol] | 6 mg | 5 mg | 4 mg | Stop | |
| (two 4 | | (two 3 mg | (one 5 mg | (one 4 mg | | |
| mg | | tablets) | tablet) | tablet) | | |
| tablets) | | | | | | |

TABLE 6B
Erdafitinib Dose Modification Rules
Based on Erdafitinib-related Toxicity

| | | Dose modification after |
|---|---|---|
| Toxicity Grade | Action | resolution of AE |
| 1 | None | Continue same dose |
| 2 | None, or consider | If interrupted, restart at |
| | interruption if the toxicity | same dose if toxicity is |
| | is considered clinically | completely resolved to |
| | significant | baseline or consider |
| | | restarting at 1 dose lower if |
| | | not completely resolved to |
| | | baseline (but resolved to |
| | | Grade 1) |
| 3 | Interrupt drug | Restart at 1 dose lower if |
| | | recovered to baseline |
| | | (to ≤Grade 1 or back to |
| | | baseline for non- |
| | | hematologic toxicity) |
| | | within 28 days; restart at 2 |
| | | doses lower if not |
| | | completely resolved to |
| | | baseline (but resolved to |
| | | Grade 1) within 28 days. |
| | | Discontinue drug if |
| | | unresolved for >28 days |
| 4 | Interrupt drug | Discontinue |

In an embodiment, the uses of, uses for or the methods of treating locally advanced or metastatic urothelial carcinoma in a subject in need thereof, in particular a cancer patient, as mentioned herein, is the use for or the treatment of a subject or a patient with luminal cluster I subtype urothelial cancer.

In an embodiment erdafitinib is administered as a pharmaceutically acceptable salt. In an embodiment, erdafitinib is administered as a pharmaceutically acceptable salt in an amount corresponding to 8 mg base equivalent or corresponding to 9 mg base equivalent.

In a preferred embodiment erdafitinib (in its base form) is administered.

In an embodiment erdafitinib is administered as a pharmaceutically acceptable salt in an amount corresponding to 8 mg base equivalent or corresponding to 9 mg base equivalent.

In an embodiment erdafitinib is administered in an amount of 8 mg of erdafitinib as such (erdafitinib base) or in an amount of 9 mg of erdafitinib as such (erdafitinib base).

The salts can be prepared by for instance reacting erdafitinib with an appropriate acid in an appropriate solvent.

Acid addition salts may be formed with acids, both inorganic and organic. Examples of acid addition salts include salts formed with an acid selected from the group consisting of acetic, hydrochloric, hydriodic, phosphoric, nitric, sulphuric, citric, lactic, succinic, maleic, malic, isethionic, fumaric, benzenesulphonic, toluenesulphonic, methanesulphonic (mesylate), ethanesulphonic, naphthalenesulphonic, valeric, acetic, propanoic, butanoic, malonic, glucuronic and lactobionic acids. Another group of acid addition salts includes salts formed from acetic, adipic, ascorbic, aspartic, citric, DL-Lactic, fumaric, gluconic, glucuronic, hippuric, hydrochloric, glutamic, DL-malic, methanesulphonic, sebacic, stearic, succinic and tartaric acids.

In an embodiment, erdafitinib is administered in the form of a solvate. As used herein, the term “solvate” means a physical association of erdafitinib with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. The term “solvate” is intended to encompass both solution-phase and isolatable solvates. Non-limiting examples of solvents that may form solvates include water, isopropanol, ethanol, methanol, DMSO, ethyl acetate, acetic acid or ethanolamine and the like.

Solvates are well known in pharmaceutical chemistry. They can be important to the processes for the preparation of a substance (e.g. in relation to their purification, the storage of the substance (e.g. its stability) and the ease of handling of the substance and are often formed as part of the isolation or purification stages of a chemical synthesis. A person skilled in the art can determine by means of standard and long used techniques whether a hydrate or other solvate has formed by the isolation conditions or purification conditions used to prepare a given compound. Examples of such techniques include thermogravimetric analysis (TGA), differential scanning calorimetry (DSC), X-ray crystallography (e.g. single crystal X-ray crystallography or X-ray powder diffraction) and Solid State NMR (SS-NMR, also known as Magic Angle Spinning NMR or MAS-NMR). Such techniques are as much a part of the standard analytical toolkit of the skilled chemist as NMR, IR, HPLC and MS. Alternatively the skilled person can deliberately form a solvate using crystallisation conditions that include an amount of the solvent required for the particular solvate. Thereafter the standard methods described above, can be used to establish whether solvates had formed. Also encompassed are any complexes (e.g. inclusion complexes or clathrates with compounds such as cyclodextrins, or complexes with metals).

All documents cited herein are incorporated by reference in their entirety.

### EXAMPLES

### Example 1: Phase 3, Multi Center, Open-Label Study (NCT03390504)

NCT03390504 is a randomized, open-label, multicenter, global phase 3 study of erdafitinib versus standard of care, consisting of chemotherapy (docetaxel or vinflunine), or anti-PD-(L)1 agent pembrolizumab, in subjects with advanced urothelial cancer and selected FGFR aberrations who have progressed on or after 1 or 2 prior treatments (cohort 1) or 1 prior treatment (cohort 2).

Subjects are assigned to Cohort 1 or Cohort 2 based upon prior treatment with an anti-PD-(L)1 agent. In Cohort 1, subjects who have received prior anti-PD-(L)1 are randomized to erdafitinib versus chemotherapy (planned enrollment approximately 280 subjects). In Cohort 2, subjects who have not received prior anti-PD-(L)1 are randomized to erdafitinib versus pembrolizumab. Cohort 1 and Cohort 2 are assessed independently.

An Independent Data Monitoring Committee (IDMC) is commissioned for this study. For each cohort, a review of the safety data is performed by an IDMC after at least 60 subjects have been enrolled in that cohort and every 6 months afterwards. A review of one predefined interim analysis for each cohort is also performed by the IDMC on both safety and efficacy data.

The Screening Phase starts with molecular screening, which is performed by a central laboratory or by review of local historical test results submitted to the sponsor for molecular eligibility assessment. Full study screening occurs after the completion of prior treatment and documentation of disease progression for subjects who meet the molecular screening criteria. The Treatment Phase extends from randomization until disease progression, intolerable toxicity, withdrawal of consent or decision by the investigator to discontinue treatment. The post-treatment Follow-up Phase extends from the End-of-Treatment Visit until the subject has died, withdraws consent, is lost to follow-up, or study completion (ie, the end of data collection timepoint has been achieved for the respective cohort), whichever comes first.

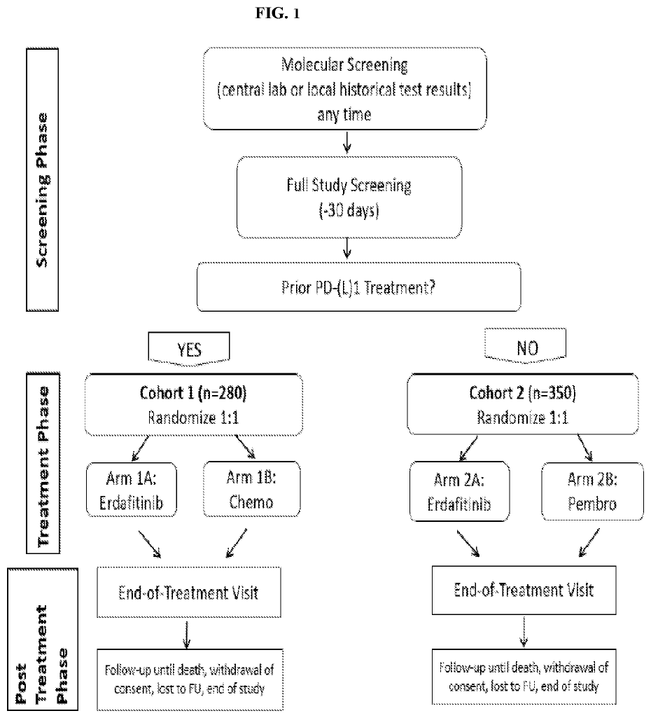

A diagram of the study design is provided in FIG. 1.

The below description relates to Cohort 1.

### Dosing

Erdafitinib is provided as a tablet for oral administration and subjects are instructed to take 8 mg orally once daily for 21 days on a 21-day cycle, until disease progression, intolerable toxicity, withdrawal of consent, or decision by the investigator to discontinue treatment.

Treatment is up-titrated to 9 mg, maintained at 8 mg, or withheld, based on phosphate level measured on Cycle 1 Day 14, and taking into account observed toxicity to that day.

Subject Population Screening for molecular eligibility may be performed at any time prior to randomization.

Full study screening for molecular eligible subjects is performed within 30 days before administration of the study drug.

### Inclusion Criteria

Each potential subject must satisfy all of the following criteria to be enrolled in the study:
1. ≥18 years of age (or the legal age of consent in the jurisdiction in which the study is taking place)
2. Histologic demonstration of transitional cell carcinoma of the urothelium. Minor components (<50% overall) of variant histology such as glandular or squamous differentiation, or evolution to more aggressive phenotypes such as sarcomatoid or micropapillary change are acceptable
3. Metastatic or surgically unresectable urothelial cancer
4. Documented progression of disease, defined as any progression that requires a change in treatment, prior to randomization
5. Prior treatment with an anti-PD-(L)1 agent as monotherapy or as combination therapy; no more than 2 prior lines of systemic treatment. Prior treatment with an anti-PD-(L)1 agent could have been given as neo-adjuvant, adjuvant, or in metastatic line of treatment as frontline or maintenance therapy, as follows:
   Together with chemotherapy or as maintenance therapy
   Together with chemotherapy in metastatic setting
   For superficial cancer (early disease/non-muscle invasive bladder cancer), or in neo-adjuvant or adjuvant setting. If these subjects did not relapse within a year of their last dose of anti-PD-(L)1, this will not be counted as a prior line of systemic treatment. These subjects will however still be eligible only for Cohort 1.

Note: Subjects who received neoadjuvant or adjuvant chemotherapy or immunotherapy and showed disease progression within 12 months of the last dose are considered to have received systemic therapy in the metastatic setting.
6. Subjects must meet appropriate molecular eligibility criteria, as determined by central laboratory screening or by local historical test results (from tissue or blood) performed at a Clinical Laboratory Improvement Amendments (CLIA)-certified or regional equivalent laboratory using the following methods: local next-generation sequencing (NGS), direct digital counting methods, or the Qiagen Therascreen FGFR Rotor-Gene Q (RGQ) reverse transcription polymerase chain reaction (RT-PCR) test. Local tests in the THOR study were all NGS-based (either tumor or blood). Tumors must have at least 1 of the following translocations: FGFR2-BICC1, FGFR2-CASP7, FGFR3-TACC3, FGFR3-BAIAP2L1, in particular FGFR2-BICC1, FGFR2-CASP7, FGFR3-TACC3_V1, FGFR-TACC3_V2, FGFR3-BAIAP2L1; or 1 of the following FGFR3 gene mutations: R248C, S249C, G370C, Y373C.
7. ECOG performance status Grade 0, 1, or 2
8. Adequate bone marrow, liver, and renal function:
a. Bone marrow function (without the support of cytokines or erythropoiesis-stimulating agent in preceding 2 weeks):
   Absolute neutrophil count (ANC) >1,500/mm3
   Platelet count >75,000/mm3 (≥100,000/mm3 for Cohort 1 subjects at sites choosing vinflunine chemotherapy)
   Hemoglobin >8.0 g/dL (without transfusion or demonstrate stability, ie; no significant decline in hemoglobin, for 2 weeks after transfusion)
b. Liver function:
   Total bilirubin ≤1.5×institutional upper limit of normal (ULN) or direct bilirubin ≤ULN for subjects with total bilirubin levels >1.5×ULN [≤1×ULN for Cohort 1 subjects at sites choosing docetaxel chemotherapy]
   Alanine aminotransferase (ALT) and aspartate aminotransferase (AST) ≤2.5× institutional ULN or ≤5× institutional ULN for subjects with liver metastases (For subjects in Cohort 1 at sites choosing docetaxel chemotherapy, both the ALT and AST values must be ≤1.5×ULN concomitant with alkaline phosphatase of ≤2.5×ULN)
c. Renal function: Creatinine clearance (CrCl) >30 mL/min either directly measured via 24-hour urine collection or calculated using the Cockcroft-Gault formula.
d. Criterion deleted per protocol amendment.
e. Phosphate: <ULN within 14 days of treatment and prior to Cycle 1 Day 1 (medical management allowed)
9. Must sign an informed consent form (ICF) (or their legally acceptable representative must sign) indicating that he or she understands the nature, significance, purpose of, procedures for, and consequences of the study and is willing to participate in the study.
10. A woman of childbearing potential who is sexually active must have a negative pregnancy test (β-human chorionic gonadotropin [β hCG]) at Screening (urine or serum).
11. Contraceptive use by men or women should be consistent with local regulations regarding the use of contraceptive methods for subject participating in clinical studies. For women of childbearing potential (defined as: fertile, following menarche and until becoming post-menopausal unless permanently sterile. Permanent sterilization methods include hysterectomy, bilateral salpingectomy and bilateral oophorectomy):
   practicing a highly effective method of contraception (failure rate of <1% per year when used consistently and correctly)
   agrees to remain on a highly effective method of contraception during the study and for at least 6 months after the last dose of study drug
   agrees to not donate eggs (ova, oocytes) for the purposes of assisted reproduction during the study and for at least 6 months after the last dose of study drug
   not breastfeeding and not planning to become pregnant during the study and for at least 6 months after the last dose of study drug For men who are sexually active with women of childbearing potential:
   agrees to use a condom with spermicidal foam/gel/film/cream/suppository
   agrees to not donate sperm during the study and for at least 6 months after the last dose of study drug
   not planning to father a child during the study or within 6 months after the last dose of study drug

### Exclusion Criteria for All Subjects

Any potential subject who meets any of the following criteria are excluded from participating in the study:
1. Treatment with any other investigational agent or participation in another clinical study with therapeutic intent within 30 days prior to randomization.
2. Active malignancies (ie, requiring treatment change in the last 24 months). The only allowed exceptions are:
   urothelial cancer.
   skin cancer treated within the last 24 months that is considered completely cured.
   localized prostate cancer with a Gleason score of 6 (treated within the last 24 months or untreated and under surveillance).
   localized prostate cancer with a Gleason score of 3+4 that has been treated more than 6 months prior to full study screening and considered to have a very low risk of recurrence.
3. Symptomatic central nervous system metastases.
4. Received prior FGFR inhibitor treatment.
5. Known allergies, hypersensitivity, or intolerance to erdafitinib or its excipients 6. Current central serous retinopathy (CSR) or retinal pigment epithelial detachment of any grade.
7. History of uncontrolled cardiovascular disease including:
a. unstable angina, myocardial infarction, ventricular fibrillation, Torsades de Pointes, cardiac arrest, or known congestive heart failure Class III-V within the preceding 3 months; cerebrovascular accident or transient ischemic attack within the preceding 3 months.
b. QTc prolongation as confirmed by triplicate assessment at screening (Fridericia; QTc >480 milliseconds).
c. Pulmonary embolism or other venous thromboembolism (VTE) within the preceding 2 months.
8. Known active AIDS (human immunodeficiency virus (HIV) infection), unless the subject has been on a stable anti-retroviral therapy regimen for the last 6 months or more, has had no opportunistic infections in the last 6 months, and has CD4 count >350.
9. Known active hepatitis B or C infection (unless polymerase chain reaction [PCR]-negative [according to local laboratory range] on all available tests for the past 6 months).
10. Not recovered from reversible toxicity of prior anticancer therapy (except toxicities which are not clinically significant such as alopecia, skin discoloration, neuropathy, hearing loss).
11. Impaired wound healing capacity defined as skin/decubitus ulcers, chronic leg ulcers, known gastric ulcers, or unhealed incisions.
12. Major surgery within 4 weeks before randomization.
13. Any condition for which, in the opinion of the investigator, participation would not be in the best interest of the subject (eg, compromise the well-being) or that could prevent, limit, or confound the protocol-specified assessments. Examples include ongoing active infection requiring systemic therapy and uncontrolled ongoing medical conditions.

In addition to the exclusion criteria listed above, any potential subject in Cohort 1 who meets the following criterion is excluded from participating in the study:
14. For those participating at sites using docetaxel: has a history of severe hypersensitivity reaction (eg, generalized rash/erythema, hypotension, bronchospasm, angioedema or anaphylaxis) to either docetaxel or to other drugs formulated with polysorbate and paclitaxel. At sites using docetaxel, subjects with evidence of interstitial lung disease or active non-infectious pneumonitis are excluded.

### Treatment Allocation

Central randomization is implemented in this study. Subjects are assigned to Cohort 1 or Cohort 2 based upon prior treatment with anti-PD-(L)1 agent. Within each cohort, subjects are randomly assigned to 1 of 2 treatment groups based on a computer-generated randomization schedule. The randomization is balanced by using randomly permuted blocks and is stratified by region (North America vs EU vs rest of world), Eastern Cooperative Oncology Group (ECOG) performance status (0 or 1 vs 2), and disease distribution (presence vs. absence of visceral metastases: lung, liver, or bone).

### Dosage and Administration

### 1. Erdafitinib

Erdafitinib is provided as tablets for oral administration. Subjects randomized to Arm 1A (see FIG. 1) are instructed to take erdafitinib orally, at a starting dose of 8 mg, once daily for 21 days in a 21-day cycle until disease progression, intolerable toxicity, withdrawal of consent or decision by the investigator to discontinue treatment. Each dose should be taken at approximately the same time each day, with or without food. The study drug is to be taken with approximately 240 mL (8 ounces) of water. The tablets should be swallowed intact and subjects should not attempt to dissolve them in water, tablets must not be broken or chewed. Subjects should avoid consuming grapefruit or Seville oranges due to CYP450 3A4/5 inhibition.

### Up-Titration Guidelines

All subjects start erdafitinib 8 mg once daily from Day 1 to Day 14 of Cycle 1. On Day 14 of Cycle 1, a blood sample is drawn to determine serum phosphate concentration.
Subjects with serum phosphate levels higher than 9.00 mg/dL (≥2.91 mmol/L) will withhold erdafitinib treatment, with at least weekly assessment of serum phosphate until it returns to less than 7.00 mg/dL (<2.25 mmol/L) while initiating treatment with a phosphate binder such as sevelamer.
Subjects with serum phosphate levels between 7.00 to 8.99 mg/dL (2.25 mmol/L to 2.90 mmol/L) should increase the erdafitinib dose to 9 mg once daily, while concurrently initiating treatment with a phosphate binder such as sevelamer.
Subjects with serum phosphate level less than 7.00 mg/dL (<2.25 mmol/L) increase the erdafitinib dose to 9 mg once daily. No concomitant treatment is required for these subjects.

If a dose is missed, then it can be taken up to 6 hours after the scheduled time; the subject may return to the normal schedule the following day. If it has been more than 6 hours since the missed dose, then that dose should be skipped and the subject should continue treatment at the scheduled time the next day. If vomiting occurred with drug administration, no replacement dose is taken and any such event that occurs up to 4 hours following dose administration must be recorded on the electronic case report form (eCRF).

TABLE 5B
Recommended dose modifications based on serum phosphate concentrations
with the use of erdafitinib after up-titration.

| Serum Phosphate | |
|---|---|
| Concentration | Erdafitinib Management^{a} |
| <6.99 mg/dL | Continue erdafitinib at current dose. |
| (1.75-2.24 mmol/L) | |
| 7.00-8.99 mg/dL | Continue erdafitinib treatment. |
| (2.25-2.90 mmol/L) | A dose reduction will be implemented for a sustained serum |
| | phosphate level of ≥7.00 mg/dL for a period of 2 months or |
| | if clinically necessary |
| | Start phosphate binder with food until phosphate |
| | level is <7.00 mg/dL. |
| 9.00-10.00 mg/dL | Withhold erdafitinib treatment until serum phosphate level |
| (>2.91-3.20 mmol/L) | returns to 7.00 mg/dL (weekly testing recommended). |
| | Re-start treatment at the same dose level. |
| | Start phosphate binder with food until serum phosphate |
| | level returns to <7.00 mg/dL. |
| | A dose reduction will be implemented for sustained serum |
| | phosphate level of ≥9.00 mg/dL for a period of 1 month or |
| | if clinically necessary |
| >10.00 mg/dL | Withhold erdafitinib treatment until serum phosphate level |
| (>3.20 mmol/L) | returns to 7.00 mg/dL (weekly testing recommended). |
| | Re-start treatment at the first reduced dose level. |
| | If serum phosphate level of ≥10.00 mg/dL is sustained |
| | for >2 weeks, erdafitinib should be discontinued permanently. |
| | Medical management as clinically appropriate. |
| Significant alteration | Erdafitinib should be discontinued permanently. |
| from baseline renal | |
| function or Grade 3 | |
| hypocalcemia due to | |
| hyperphosphatemia. | |

^{a}For phosphate concentrations >5.5 mg/dL, restrict phosphate intake to 600-800 mg/day.

The recommended dose modifications for adverse reactions including hyperphosphatemia are listed in Table 6C.

TABLE 6C
Erdafitinib dose reduction schedule

| | | 1^{st} | 2^{nd} | 3^{rd} | 4^{th} | 5^{th} |
|---|---|---|---|---|---|---|
| | | dose | dose | dose | dose | dose |
| Dose | | reduction | reduction | reduction | reduction | reduction |
| 9 mg | [symbol] | 8 mg | 6 mg | 5 mg | 4 mg | Stop |
| (three | | (two 4 mg | (two 3 mg | (one 5 mg | (one 4 mg | |
| 3 mg | | tablets) | tablets) | tablet) | tablet) | |
| tablets) | | | | | | |
| 8 mg | [symbol] | 6 mg | 5 mg | 4 mg | Stop | |
| (two | | (two 3 mg | (one 5 mg | (one 4 mg | | |
| 4 mg | | tablets) | tablet) | tablet) | | |
| tablets) | | | | | | |

### 2. Vinflunine or Docetaxel

Subjects randomized to chemotherapy in Arm 1B (see FIG. 1) receive vinflunine 320 mg/m2 as a 20-minute intravenous infusion once every 3 weeks or docetaxel 75 mg/m2 as a 1-hour intravenous infusion every 3 weeks. Treatment with either agent will be administered until disease progression, intolerable toxicity, withdrawal of consent or decision by the investigator to discontinue treatment.

The choice of which chemotherapy regimen to use at each site will be determined by the investigator.

In cases where toxicity does not resolve to Grade 0-1 within 2 weeks after the last infusion of vinflunine or 4 weeks after the last infusion of docetaxel, study treatment should be discontinued after consultation with the sponsor. With investigator and sponsor agreement, subjects with a laboratory adverse event still at Grade 2 after 4 weeks may continue in the study only if asymptomatic and stable.

### 2.1. Vinflunine

Vinflunine, 320 mg/m2, should be administered after all procedures and assessments for that day have been completed. The body surface area (BSA) in m2 should be calculated per local institutional practice. Consult the vinflunine summary of product characteristics for instructions on preparation and administration of the vinflunine infusion fluid and recommendations for co-medication (ie, laxatives and dietary measures including oral hydration are recommended from Day 1 to Day 7 after each vinflunine administration).

### 2.1.1. Vinflunine Dose Modifications

In case of WHO/ECOG performance status (PS) of ≥1 or PS of 0 and prior pelvic irradiation, vinflunine treatment should be started at the dose of 280 mg/m2. In the absence of any hematological toxicity during the first cycle causing treatment delay or dose reduction, the dose will be increased to 320 mg/m2 every 3 weeks for the subsequent cycles.

In subjects with moderate renal impairment (40 ml/min≤CrCl≤60 ml/min), the recommended dose is 280 mg/m2 given once every 3 weeks. In subjects with severe renal impairment (20 mL/min≤CrCl≤40 mL/min), the recommended dose is 250 mg/m2 given once every 3 weeks.

The recommended dose of vinflunine is 250 mg/m2 given once every 3 weeks in subjects with mild liver impairment (Child-Pugh Grade A) or in subjects with a prothrombin time ≥60% normal value and 1.5×ULN<Bilirubin≤3×ULN and presenting at least one of the following criteria:
transaminases >ULN or gamma-glutamyltransferase >5×ULN.

The recommended dose of vinflunine is 200 mg/m2 given once every 3 weeks in patients with moderate liver impairment (Child-Pugh grade B) or in patients with a prothrombin time ≥50% normal value and bilirubin >3×ULN and transaminases >ULN and gamma-glutamyltransferase >ULN.

The doses recommended in subjects ≥75 years old are as follows:
in subjects at least 75 years old but less than 80 years, the dose of vinflunine to be given is 280 mg/m2 every 3 weeks.
in subjects 80 years old and beyond, the dose of vinflunine to be given is 250 mg/m2 every 3 weeks.

In subjects who initiate vinflunine at 280 mg/m2 and who experience an AE requiring dose modification, the dose should be reduced to 250 mg/m2 following the first occurrence and resolution, and discontinued following a second occurrence. In subjects who initiate vinflunine at 250 mg/m2 and who experience an AE requiring dose modification, the dose should be reduced to 225 mg/m2 following the first occurrence and resolution, and discontinued following a second occurrence.

Cases of posterior reversible encephalopathy syndrome (PRES) have been observed after administration of vinflunine. The typical clinical symptoms are, as follows: neurological (headache, confusion, seizure, visual disorders), systemic (hypertension), and gastrointestinal (nausea, vomiting). Radiological signs are white matter abnormalities in the posterior regions of the brain. Vinflunine should be discontinued in subjects who develop neurological signs of PRES.

For all grades of neutropenia lasting ≤7 days, hold vinflunine administration until neutrophils recover to >1500 cells/mm3. For thrombocytopenia Grades 1, 2, 3, hold vinflunine until platelets recover to >100,000 cells/mm3. For anemia Grades 1, 2, 3, hold vinflunine until resolution to Grade 1 or baseline. Specific dose modifications for subjects receiving vinflunine are recommended in the below Table 7A. Dose modifications for vinflunine should also be considered according to local product labels.

TABLE 7A
Vinflunine Dose Adjustments for Drug-related Adverse Events

| | Dose Adjustment | | | | |
|---|---|---|---|---|---|
| | Initial Dose: | | | Initial Dose: | |
| | Vinflunine 320 mg/m² | | | Vinflunine 280 mg/m² | |
| | First | Second | Third | First | Second |
| Toxicity | Occurrence | Occurrence | Occurrence | Occurrence | Occurrence |
| Neutropenia Grade 4 lasting >7 days | Vinflunine | Vinflunine | Discontinue | Vinflunine | Discontinue |
| (hold treatment until neutrophils | 280 mg/m² | 250 mg/m² | treatment | 250 mg/m² | treatment |
| >1500 cells/mm²) | | | | | |
| Thrombocytopenia Grade 4 (hold | | | | | |
| treatment until platelets >100,000 | | | | | |
| cells/mm²) | | | | | |
| Anemia Grade 4 (hold treatment | | | | | |
| until Grade 1 or baseline) | | | | | |
| All other Grade 3 or 4 toxicities | | | | | |
| (hold treatment until Grade 1 or | | | | | |
| baseline) | | | | | |
| Neutropenic fever ( Temperature | | | | | |
| ≥100.5 °F and ANC ≤1000/L) | | | | | |
| Mucositis or constipation Grade 2 | | | | | |
| ≥25 days or Grade ≥3 any duration ^{a} | | | | | |
| Cardiac ischemia in subjects with | Discontinue | N/A | N/A | Discontinue | N/A |
| prior history of myocardial infarction | treatment | | | treatment | |
| or angina pectoris | | | | | |

^{a} NCI CTC Grade 2 constipation is defined as requiring laxatives, Grade 3 as an obstipation requiring manual evacuation or enema, Grade 4 as an obstruction or toxic megacolon. Mucositis Grade 2 is defined as “moderate”, Grade 3 as “severe” and Grade 4 as “life-threatening”

### 2.2. Docetaxel

Docetaxel, 75 mg/m2, should be administered after all procedures and assessments for that day have been completed. The body surface area (BSA) in m2 should be calculated per local institutional practice. All subjects should be premedicated with oral corticosteroids, such as dexamethasone 16 mg per day (eg, 8 mg twice daily) for 3 days starting 1 day prior to docetaxel administration in order to reduce the incidence and severity of fluid retention as well as the severity of hypersensitivity reactions. The appropriate premedication regimen may be determined by the investigator.

Cystoid macular edema has been reported in subjects treated with docetaxel. Subjects with impaired vision should undergo a prompt and complete ophthalmologic examination. In case cystoid macular edema is diagnosed, docetaxel treatment should be discontinued and appropriate treatment initiated.

Consult the docetaxel prescribing information for instructions on preparation and administration of the docetaxel infusion fluid, and recommendations for co-medication.

### 2.2.1. Docetaxel Dose Modifications

Docetaxel should not be given to subjects with bilirubin >1×ULN, or to subjects with AST or ALT >1.5×ULN with concomitant alkaline phosphatase >2.5×ULN. Subjects with elevations of bilirubin or abnormalities of transaminase concurrent with alkaline phosphatase are at increased risk for the development of Grade 4 neutropenia, febrile neutropenia, infections, severe thrombocytopenia, severe stomatitis, severe skin toxicity, and toxic death. Docetaxel should also not be given to subjects with a neutrophil count of <1500 cells/mm3. Severe fluid retention has been reported following docetaxel therapy. Subjects should be premedicated with oral corticosteroids prior to each docetaxel administration to reduce the incidence and severity of fluid retention. Subjects with preexisting effusions should be monitored closely from the first dose for the possible exacerbation of the effusions. Subjects developing peripheral edema may be treated with standard measures, eg, salt restriction, oral diuretic(s).

For all grades of neutropenia lasting ≤7 days, hold docetaxel until neutrophils recover to >1500 cells/mm3. For thrombocytopenia Grades 1, 2, 3, hold docetaxel until platelets recover to >100,000 cells/mm3. For anemia Grades 1, 2, 3, hold docetaxel until resolution to Grade 1 or baseline. Dose modifications for subjects receiving docetaxel are recommended in the below Table 8A.

Dose modifications for docetaxel should also be considered according to local product labels.

TABLE 8A
Docetaxel Dose Modifications for Drug-related Adverse Events

| | | | | |
|---|---|---|---|---|
| | | | | Treatment |
| Grade | Occurrence | Hold Treatment | Dose Modification | Discontinuation |

Peripheral Neuropathy

| | | | |
|---|---|---|---|
| Grade 1, 2 | No | 60 mg/m² | N/A |
| Grade 3, 4 | Yes | N/A | Discontinue upon |
| | | | onset |

Neutopenic Fever (defined as T ≥100.5 °F and ANC ≤1000/L)
Neutropenia Grade 4 lasting more than 7 days

| | | | |
|---|---|---|---|
| First | Hold treatment until | Docetaxel: 60 mg/m² | |
| occurrence | neutrophils recover to | | |
| | ≥1500/L | | |
| Second | Hold treatment until | Docetaxel: 50 mg/m² | Consider treatment |
| occurrence | neutrophils recover to | | discontinuation |
| | ≥1500/L | | |
| Third | Yes | N/A | Yes |
| occurrence | | | |

Thrombocytopenia

| | | | | |
|---|---|---|---|---|
| Grade 4 | First | Hold treatment until | Docetaxel: 60 mg/m² | Consider treatment |
| | occurrence | platelets recover to | | discontinuation |
| | | >100,000 cells/mm² | | |
| | Second | Hold treatment until | Docetaxel: 50 mg/m² | Consider treatment |
| | occurrence | platelets recover to | | discontinuation |
| | | >100,000 cells/mm² | | |
| | Third | Yes | N/A | Yes |
| | occurrence | | | |

Anemia

| | | | | |
|---|---|---|---|---|
| Grade 4 | First | Hold treatment until | Docetaxel: 60 mg/m² | Consider treatment |
| | occurrence | resolution to Grade 1 or | | discontinuation |
| | | baseline | | |
| | Second | Hold treatment until | Docetaxel: 50 mg/m² | Consider treatment |
| | occurrence | resolution to Grade 1 or | | discontinuation |
| | | baseline | | |
| | Third | Yes | N/A | Yes |
| | occurrence | | | |

Non-hematologic or other hematologic toxicity not described above

| | | | | |
|---|---|---|---|---|
| Grade 3, 4 | First | Hold treatment until | Docetaxel: 60 mg/m² | Consider treatment |
| | occurrence | resolution to Grade 0-1 or | | discontinuation |
| | | baseline | | |
| | Second | Hold treatment until | Docetaxel: 50 mg/m² | Consider treatment |
| | occurrence | resolution to Grade 0-1 or | | discontinuation |
| | | baseline | | |
| | Third | Yes | N/A | Yes |
| | occurrence | | | |

### Efficacy Analyses

### Primary Endpoint

The primary endpoint is OS. The primary efficacy analysis is based on the Intent-to-Treat (ITT) population that includes all randomized subjects in each cohort. The Kaplan-Meier method is used to estimate the distribution of OS for each treatment group within each cohort. The stratified log-rank test is used to compare survival curves of OS between the 2 treatment arms. The stratification factors to be used in the analysis are as follows: region (North America vs EU vs rest of world), ECOG performance status (0 or 1 vs 2), and disease distribution (presence vs absence of visceral metastases: lung, liver, or bone). Pre-specified strata combining will be implemented when some strata have too few OS events. Additionally, the hazard ratio for erdafitinib relative to the control and its associated 95% CI is calculated based on the Cox proportional hazards model.

### Secondary Efficacy Endpoints

The secondary efficacy endpoints are PFS, ORR (achievement of objective response), and Safety. Secondary end points also included DOR, change from baseline in patient reported outcomes (Functional Assessment of Cancer Therapy—Bladder Cancer, Patient-Global Impression of Severity, and the European Quality of Life—5 Dimensions—5 Levels). PFS is analyzed the same as OS. Kaplan-Meier method is used to estimate the distribution of DOR. ORR is analyzed by Cochran-Mantel-Haenszel chi-square test for randomized subjects with measurable disease at baseline. The analyses of the secondary efficacy endpoints include all randomized subjects (ITT subjects) unless otherwise specified. The family-wise type 1 error is strongly controlled at 5% (2-sided) for the secondary efficacy endpoints.

### Subgroup Analyses

The primary efficacy and secondary efficacy endpoints are analyzed by subgroups.

### Assessments (Cohort 1)

Assessment of responses for solid tumors were investigator-assessed by RECIST v1.1 and performed every 6 weeks for the first 6 months and then every 12 weeks for the next 6 months and beyond. Adverse events were graded according to the National Cancer Institute Common Terminology Criteria for Adverse Events version 4.03. Ophthalmologic examination at baseline included an Amsler grid test, optical coherence tomography scan (OCT), and ophthalmologic evaluation. An Amsler grid test was conducted at every cycle. Repeat OCT was done as clinically indicated based on the Amsler grid test or clinical assessment.

### Safety Analyses

### Adverse Events

The verbatim terms used in the case report form (CRF) by investigators to identify adverse events are coded using the Medical Dictionary for Regulatory Activities (MedDRA). Treatment-emergent adverse events are adverse events with onset during the Treatment Phase or that are a consequence of a preexisting condition that has worsened since baseline. All reported adverse events are included in the analysis. For each adverse event, the percentage of subjects who experience at least 1 occurrence of the given event is summarized by treatment group. In addition, comparisons between treatment groups will be provided if appropriate.

Adverse event information will be graded using the National Cancer Institute Common Terminology Criteria for Adverse Events (NCI-CTCAE) Version 4.03. Adverse event severity is a clinical determination of the intensity of an adverse event. The severity assessment for an adverse event or serious adverse event should be completed using the NCICTCAE, Version 4.03. Any adverse event or serious adverse event not listed in the NCI-CTCAE, Version 4.03 will be graded according to the investigator clinical judgment by using the standard grades as follows:
Grade 1: Mild; asymptomatic or mild symptoms; clinical or diagnostic observations only; intervention not indicated.
Grade 2: Moderate; minimal, local or noninvasive intervention indicated; limiting age-appropriate instrumental activities of daily living.
Grade 3: Severe or medically significant but not immediately life-threatening; hospitalization or prolongation of hospitalization indicated; disabling; limiting self-care activities of daily living.
Grade 4: Life-threatening consequences; urgent intervention indicated.
Grade 5: Death related to adverse event.

The investigator should use clinical judgment in assessing the severity of events not directly experienced by the subject (eg, laboratory abnormalities).

### Statistical Analysis (Cohort 1)

The study was designed to have at least 85% power to detect a hazard ratio of 0.65, corresponding to a 53% increase in median overall survival for the erdafitinib group versus the chemotherapy group, with a two-sided Type I error level of 0.05; one interim analysis of both efficacy and futility was planned at approximately 65% information fraction (about 136 out of a total of 208 deaths). The enrollment of approximately 280 patients was determined to be sufficient to provide the required statistical power. O'Brien-Fleming boundaries were applied, implemented by the Lan-DeMets spending function for a total Type I error of 0.05. Early stopping for efficacy would be warranted if the two-sided p-value at the interim analysis was less than 0.019 based on the observed 75% information fraction (ie, 155 deaths) at the clinical cut-off date. Stopping for futility was possible if the hazard ratio at the interim analysis exceeded 1.0, considering the totality of the data.

Key secondary endpoints were part of a hierarchical testing strategy to strongly control the overall family-wise Type I error rate at 0.05 (two-sided). Descriptive subgroup analyses were conducted, but with no adjustment for multiplicity. 95% confidence intervals are presented but should not be used in place of a hypothesis test. Efficacy analyses used the intention-to-treat population, comprising all patients randomized. Safety analyses used the safety population, comprising all patients who received at least one dose of study treatment. The distribution of overall survival and progression-free survival for each treatment arm was summarized using the Kaplan-Meier method and compared with a log-rank test. The estimated hazard ratio with 95% confidence interval summarizing the magnitude of the benefit of erdafitinib relative to chemotherapy was derived from a Cox proportional hazards model with treatment as the sole independent variable. The Cochran-Mantel-Haenszel method was used to compare the distribution of objective response between treatment groups, including an estimate of the relative risk with 95% confidence interval.

### Results Part 1

TABLE 9A
Summary of Demographics and Disease Characteristics

| | Erdafitinib | Chemotherapy |
|---|---|---|
| Characteristic | (n = 136) | (n = 130) |
| Age, median (range), years | 66 (32-85) | 69 (35-86) |
| Men, n (%) | 96 (70.6) | 94 (72.3) |
| Race, n (%) | | |
| White | 81 (59.6) | 63 (48.5) |
| Asian | 32 (27.2) | 40 (30.8) |
| Black or African American | 0 | 1 (0.8) |
| Multiple | 0 | 1 (0.8) |
| Not reported | 18 (13.2) | 25 (19.2) |
| Presence of visceral | 101 (74.3) | 97 (74.6) |
| metastases, n (%) | | |
| Liver | 31 (22.8) | 38 (29.2) |
| ECOG PS 0-1, n (%) | 124 (91.2) | 117 (90) |
| Primary tumor upper tract, n (%) | 41 (30.1) | 48 (36.9) |
| PD-L1 low (CPS < 10), n (%) | 89 (92.7)^{b} | 68 (86.1)^{b} |
| FGFRalt, n (%)^{c} | (n = 135) | (n = 129) |
| Mutations | 108 (79.4) | 107 (82.3) |
| Fusions | 25 (18.4) | 19 (14.6) |
| Mutations and fusions | 2 (1.5) | 3 (2.3) |
| Prior lines of systemic therapy | | |
| 1 line | 45 (33.1) | 33 (25.4) |
| 2 lines | 90 (66.2) | 97 (74.6) |

^{a}One patient in the erdafitinib group had 3 prior line of systemic therapy.
^{b}For PD-L1 status, percentage is based on patients with available data (n = 96 for erdafitinib and n = 79 for chemotherapy).
^{c}All patients enrolled had FGFR3 alterations. Two patients were subsequently identified as false positives; they were included in the ITT population.
CPS, combined positive score.

Patient baseline characteristics were generally balanced between treatment arms. Most patients had PD-L1 low expression status. 33% of patients in the erdafitinib group and 25% in the chemotherapy group had 1 prior-line of systemic therapy. 66% and 75% had 2 lines of prior therapy, respectively.

TABLE 10A
Summary of baseline Characteristics

| Patients receiving prior | Erdafitinib | Chemotherapy |
|---|---|---|
| therapy, n (%) | (n = 136) | (n = 130) |
| 1 line of prior systemic therapy | 45 (33.1) | 33 (25.4) |
| Chemotherapy + anti-PD-(L)1^{a} | 33 (24.3) | 15 (11.5) |
| Anti-PD-(L)1^{b} | 11 (8.1) | 16 (12.3) |
| Chemotherapy | 1 (0.7) | 2 (1.5) |
| 2 lines of prior systemic therapy | 90 (66.2) | 97 (74.6) |
| First line of therapy | | |
| Chemotherapy | 77 (56.6) | 76 (58.5) |
| Chemotherapy + anti-PD-(L)1 | 6 (4.4) | 10 (7.7) |
| Other | 7 (5.1) | 11 (8.5) |
| Second line of therapy | | |
| Anti-PD-(L)1 | 76 (55.9) | 76 (58.5) |
| Chemotherapy | 10 (7.4) | 14 (10.8) |
| Other | 4 (2.9) | 7 (5.4) |

^{a}Includes patients who received other therapy in addition to chemotherapy + anti-PD-(L)1.
^{b}Includes patients who received other therapy in addition to anti-PD-(L)1.

### 1 Line of Therapy:

Erda: 33 patients (24.3%) received both chemotherapy and anti-PD-(L)1
Chemo: 16 patients (12.3%) received anti-PD-(L)1 without chemotherapy; patients (11.5%) received both chemotherapy and anti-PD-(L)1

### 2 Lines of Therapy:

First-line:
   Erda: 77 patients (56.6%) received chemotherapy without anti-PD-(L)1
   Chemo: 76 patients (58.5%) received chemotherapy without anti-PD-(L)1
Second-line:
   Erda: 76 patients (55.9%) received anti-PD-(L)1 without chemotherapy
   Chemo: 78 patients (60.0%) received anti-PD-(L)1 without chemotherapy

For patients who received 1 prior line of therapy:
There was a mix of patients who received a checkpoint inhibitor (anti-PD-(L)1) as maintenance or in combination with chemotherapy vs those who received a checkpoint inhibitor (anti-PD-(L)1) alone (without chemotherapy)

For patients who received 2 prior lines of therapy:
Most patients received chemotherapy in the first-line setting and checkpoint inhibitor (anti-PD-(L)1) in the second-line setting

TABLE 11A
Summary of FGFR Genetic Alterations; Cohort 1 ITT
Analysis Set (Study 42756493-BLC3001)

| | Erdafitinib | Chemotherapy | Total |
|---|---|---|---|
| Analysis Set: ITT | 136 | 130 | 266 |
| Subjects with any | 135 (99.3%) | 129 (99.2%) | 264 (99.2%) |
| FGFR alterations | | | |
| Mutations (excluding | 108 (79.4%) | 107 (82.3%) | 215 (80.8%) |
| fusions) | | | |
| FGFR3 R248C and | 0 | 1 (0.8%) | 1 (0.4%) |
| FGFR3-TACC3_V1 | | | |
| FGFR3 S249C | 65 (47.8%) | 59 (45.4%) | 124 (46.6%) |
| FGFR3 Y373C | 18 (13.2%) | 27 (20.8%) | 45 (16.9%) |
| FGFR3 R248C | 17 (12.5%) | 8 (6.2%) | 25 (9.4%) |
| FGFR3 G370C | 5 (3.7%) | 8 (6.2%) | 13 (4.9%) |
| FGFR3 R248C and | 2 (1.5%) | 0 | 2 (0.8%) |
| FGFR3 Y373C | | | |
| FGFR3 S249C and | 1 (0.7%) | 1 (0.8%) | 2 (0.8%) |
| FGFR3 Y373C | | | |
| FGFR3 G370C and | 0 | 1 (0.8%) | 1 (0.4%) |
| FGFR3 Y373C | | | |
| FGFR3 R248C and | 0 | 1 (0.8%) | 1 (0.4%) |
| FGFR3 S249C | | | |
| FGFR3 G370C and | 0 | 1 (0.8%) | 1 (0.4%) |
| FGFR3 S249C and FGFR3 | | | |
| Y373C | | | |
| FGFR3 R248C and | 0 | 1 (0.8%) | 1 (0.4%) |
| FGFR3 S249C and FGFR3 | | | |
| Y373C | | | |
| Fusions (excluding | 25 (18.4%) | 19 (14.6%) | 44 (16.5%) |
| mutations) | | | |
| FGFR3-TACC3_V1 | 16 (11.8%) | 10 (7.7%) | 26 (9.8%) |
| FGFR3-TACC3 | 5 (3.7%) | 6 (4.6%) | 11 (4.1%) |
| FGFR3-TACC3_V3 | 3 (2.2%) | 1 (0.8%) | 4 (1.5%) |
| FGFR3-BAIAP2L1 | 0 | 1 (0.8%) | 1 (0.4%) |
| FGFR3-BAIAP2L1 and | 0 | 1 (0.8%) | 1 (0.4%) |
| FGFR3-TACC3_V1 | | | |
| FGFR3-TACC3_V1 | 1 (0.7%) | 0 | 1 (0.4%) |
| and FGFR3-TACC3_V3 | | | |
| Mutations and fusions | 2 (1.5%) | 3 (2.3%) | 5 (1.9%) |
| FGFR3 S249C and | 2 (1.5%) | 0 | 2 (0.8%) |
| FGFR3-TACC3_V1 | | | |
| FGFR3 S249C and | 0 | 1 (0.8%) | 1 (0.4%) |
| FGFR3-TACC3_V3 | | | |
| FGFR3 Y373C and | 0 | 1 (0.8%) | 1 (0.4%) |
| FGFR3-TACC3_V1 | | | |

Percentages are based on the number of subjects in analysis set of the corresponding treatment group.

FGFR genetic alterations from local laboratory are used if no central laboratory data available.

### Primary Efficacy Endpoint:

Overall survival (OS), defined as the time from randomization to the date of death due to any cause is the primary efficacy endpoint.
In Cohort 1, an IA was pre-planned to assess both efficacy and futility after approximately 65% information fraction had been achieved. The IA occurred at approximately 75% information fraction and the significance level was determined using the O'Brien-Fleming (OBF) alpha-spending function to control the Type I error at the 0.05 significance level (2-sided). The efficacy boundary p-value was determined to be 0.019 (2-sided). Based on these interim results demonstrating superiority of erdafitinib over chemotherapy, the Independent Data Monitoring Committee (IDMC) recommended to stop the trial, unblind the study and allow crossover to erdafitinib for patients randomized to the chemotherapy arm.

At the clinical cut-off (CCO) date, a total of 266 subjects (Erdafitinib: 136; chemotherapy: 130) had been randomized. Among the randomized subjects, 247 subjects (Erdafitinib: 135; chemotherapy: 112) had received at least one dose of study drug. Subjects randomized but never treated discontinued the study most commonly due to withdrawal by subject (10 subjects) and death (9 subjects). As of the data cut, 208 (Erdafitinib: 106; chemotherapy: 102) of the 247 treated subjects had discontinued treatment. The median follow-up was 15.9 months.

The analyses were unstratified for OS and PFS. ORR analysis was stratified by ECOG performance status (PS) per the pooling algorithm.

TABLE 12
Summary of OS, PFS, ORR

| | Erdafitinib | Chemotherapy |
|---|---|---|
| | (N = 136) | (N = 130) |
| Overall Survival (OS) | | |
| Number of events (%) | 77 (56.6%) | 78 (60.0%) |
| Median, months (95% CI) | 12.06 (10.28, 16.36) | 7.79 (6.54, 11.07) |
| HR (95% CI) | 0.64 (0.47, 0.88) | |
| P-value | 0.0050 | |
| Progression-free survival (PFS) | | |
| Number of events (%) | 101 (74.3%) | 90 (69.2%) |
| Median, months (95% CI) | 5.55 (4.40, 5.65) | 2.73 (1.81, 3.68) |
| HR (95% CI) | 0.58 (0.44, 0.78) | |
| P-value | 0.0002 | |
| Objective response rate (ORR) | | |
| Complete response, CR (%) | 9 (6.6%) | 1 (0.8%) |
| Partial response, PR (%) | 53 (39.0%) | 14 (10.8%) |
| ORR (CR + PR) | 62 (45.6%) | 15 (11.5%) |

| | |
|---|---|
| Relative Risk (RR), (95% CI) | 3.94 (2.37, 6.57) |
| P-value | <0.001 |

Note:
All p-values reported are 2-sided;
CI = confidence interval

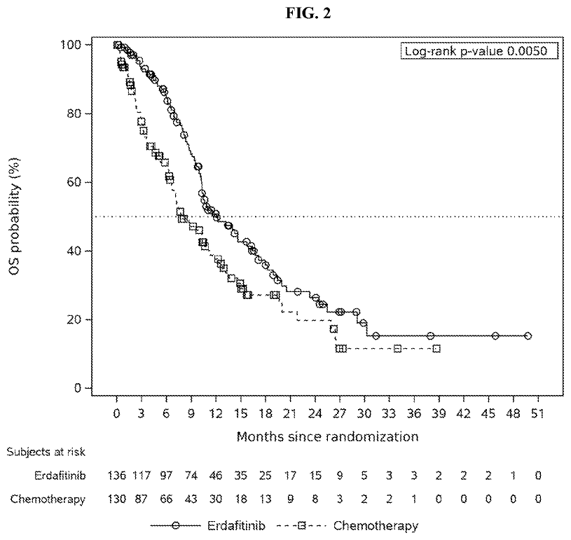

A total of 155 OS events (Erdafitinib: 77; chemotherapy: 78) had occurred in the ITT population at the data cut, corresponding to approximately 75% information fraction at the Interim analysis (IA). The Kaplan-Meier (K-M) estimate of the median OS was 12.1 (95% CI: 10.3, 16.4) months for Erdafitinib and 7.8 (95% CI: 6.5, 11.1) months for chemotherapy. A hazard ratio (HR) of 0.64 (95% CI: 0.47, 0.88) was observed based on a (non-stratified) Cox proportional hazards model, representing a 36.0% reduction in the risk of death for the Erdafitinib arm (2-sided log-rank test p-value=0.0050). The superiority of Erdafitinib relative to chemotherapy, in terms of OS, was established since the pre-determined 2-sided efficacy boundary p-value of 0.019, based on OBF alpha-spending function, was crossed. FIG. 2 shows the K-M OS curves for the two treatment arms. The results are generally consistent across clinically meaningful subgroups including age group, FGFR alteration type, baseline ECOG PS, presence/absence of visceral metastases and PD-L1 status.

### Key Secondary Efficacy Endpoints

### Progression-Free Survival

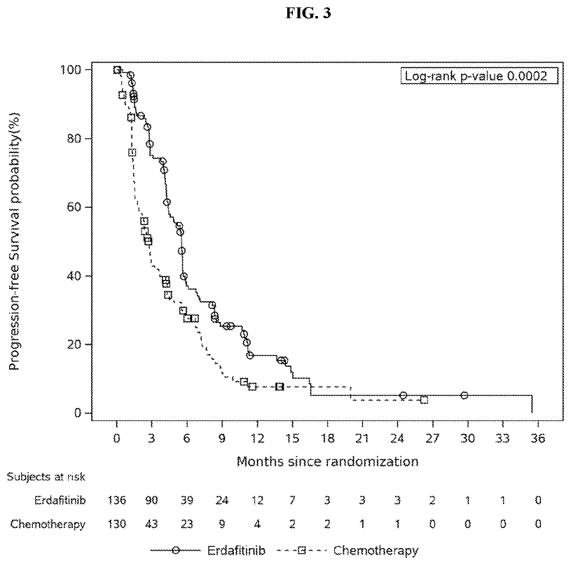

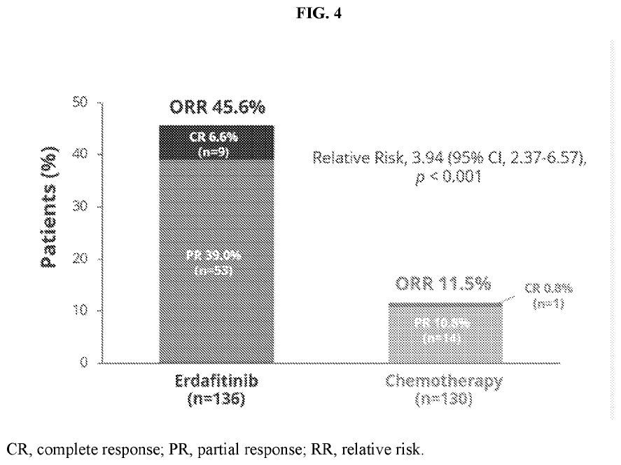

A total of 191 progression-free survival (PFS) events (Erdafitinib: 101; chemotherapy: 90) accrued as of the data cut. The K-M estimate of the median PFS was 5.6 (95% CI: 4.4, 5.7) months for Erdafitinib and 2.7 (95% CI: 1.8, 3.7) months for chemotherapy. A HR of 0.58 (95% CI: 0.44, 0.78) was observed based on a (non-stratified) Cox proportional hazards model, representing a 42.0% reduction in the risk of progression or death for the Erdafitinib arm (2-sided log-rank test p-value=0.0002). The superiority of Erdafitinib relative to chemotherapy, in terms of PFS, was established since the pre-determined 2-sided efficacy boundary p-value of 0.019 was crossed. FIG. 3 shows the K-M PFS curves for the two treatment arms.

### Objective Response Rate

In the ITT population, ORR, defined as the proportion of subjects with a best response of partial or complete response, was 45.6% (62/136) with 95% CI: 37.0%, 54.3% and 11.5% (15/130) with 95% CI: 6.6%, 18.3%, respectively, for Erdafitinib and chemotherapy, with an observed relative risk of 3.94 (95% CI: 2.37, 6.57) based on Cochran-Mantel-Haenszel (CMH) test, representing approximately a four-fold higher probability of achieving an objective response for the Erdafitinib arm (2-sided p-value<0.001) compared to the chemotherapy arm (Table 13. The superiority of Erdafitinib relative to chemotherapy, in terms of ORR, was established since the pre-determined 2-sided efficacy boundary p-value of 0.019, based on OBF alpha spending function, was crossed. Median duration of response (DOR) was 4.9 (95% CI: 3.8, 7.5) vs. 5.6 (95% CI: 2.1, 6.0) months for Erdafitinib and chemotherapy, respectively.

TABLE 13
Summary of Best Overall Response-Stratified Analysis (Interactive Web
Response Systems (IWRS)-defined stratification factors); Cohort 1 ITT
Analysis Set (Study 42756493-BLC3001)

| | Erdafitinib | Chemotherapy |
|---|---|---|
| Analysis set: ITT | 136 | 130 |
| Best overall response | | |
| Complete response (CR) | 9 (6.6%) | 1 (0.8%) |
| Partial response (PR) | 53 (39.0%) | 14 (10.8%) |
| Stable disease (SD)^{a} | 50 (36.8%) | 41 (31.5%) |
| Progressive disease (PD) | 14 (10.3%) | 31 (23.8%) |
| Not evaluable (NE) | 10 (7.4%) | 43 (33.1%) |
| Objective response rate (CR + PR) | 62 (45.6%) | 15 (11.5%) |
| Relative risk (95% CI)^{b} | 3.94 (2.37, 6.57) | |
| P-value^{b} | <.001 | |
| Disease control rate (CR + PR + | 112 (82.4%) | 56 (43.1%) |
| SD) | | |
| Relative risk (95% CI)^{b} | 1.91 (1.55, 2.35) | |
| P-value^{b} | <0.001 | |

Key: CI = confidence interval; NE = not estimable.
^{a}Minimum duration requirement for SD is 6 weeks from the date of randomization. Stable disease includes subjects with no measurable disease at baseline and their best response was Non-CR/Non-PD.
^{b}Relative risk, 95% CI, and p-value are estimated using Cochran-Mantel-Haenszel (CMH) test stratified by ECOG performance status (0 or 1 vs 2).

### Cohort 1 Safety Summary

Among the 247 subjects in the safety analysis set, 133 (98.5%) in the Erdafitinib arm and 109 (97.3%) in the chemotherapy arm experienced at least one treatment-emergent adverse event (TEAE).
Grade 3-4 TEAEs were reported by 85 (63.0%) subjects in the Erdafitinib arm and 72 (64.3%) subjects in the chemotherapy arm.
Serious TEAEs were reported by 56 (41.5%) subjects in the Erdafitinib arm and 47 (42.0%) subjects in the chemotherapy arm.
In the Erdafitinib arm, 19 (14.1%) subjects discontinued treatment due to TEAEs while 20 (17.9%) subjects discontinued treatment due to TEAEs in the chemotherapy arm.
In the Erdafitinib arm, 19 (14.1%) subjects discontinued treatment due to adverse events of any cause while 20 (17.9%) of subjects discontinued treatment due to adverse events of any cause in the chemotherapy arm.
TEAEs leading to death were reported by 6 (4.4%) subjects in the Erdafitinib arm and 7 (6.2%) subjects in the chemotherapy arm.
Clinically important TEAEs for Erdafitinib were reported by 125 (92.6%) subjects in the Erdafitinib arm: hyperphosphatemia (108, 80.0%), stomatitis (65, 48.1%), dry mouth (53, 39.3%), onycholysis (31, 23.0%), dry skin (31, 23.0%), Palmar-plantar erythrodysaesthesia syndrome (41, 30.4%) and dry eye (23, 17.0%). Fewer subjects in the chemotherapy arm had these events reported.

Central serous retinopathy (CSR), a known class effect of FGFR inhibitors, is an adverse event of special interest. TEAEs of CSR were reported by 23 (17.0%) subjects in the Erdafitinib arm. No subjects in the chemotherapy arm reported CSR events.

### In the Erdafitinib Group:

18 patients (13.3%) had treatment-related serious AEs
1 treatment-related death occurred^{c}
AEs with erdafitinib were mostly manageable with dose modifications and supportive care

### In the Chemotherapy Group:

27 patients (24.1%) had treatment-related serious AEs
6 treatment-related deaths occurred^{f}

TABLE 14
Safety results

| | Erdafitinib (n = 135) | |
|---|---|---|
| Patients with AEs, n (%)^{a} | Any grade | Grade 3-4 |
| ≥1 treatment-related AE | 131 (97.0) | 62 (45.9) |
| Hyperphosphatemia | 106 (78.5) | 7 (5.2) |
| Diarrhea | 74 (54.8) | 4 (3.0) |
| Stomatitis | 62 (45.9) | 11 (8.1) |
| Dry mouth | 52 (38.5) | 0 |
| PPE syndrome | 41 (30.4) | 13 (9.6) |
| Onycholysis | 31 (23.0) | 8 (5.9) |
| Patients who discontinued study | | |
| treatment, n (%) | | |
| Discontinuation due to treatment- | 11 (8.1%)^{b} | |
| related AEs | | |
| | Chemotherapy (n = 112) | |
| Patients with AEs, n (%)^{d} | Any grade | Grade 3-4 |
| ≥1 treatment-related AE | 97 (86.6) | 52 (46.4) |
| Anemia | 31 (27.7) | 7 (6.3) |
| Alopecia | 24 (21.4) | 0 |
| Nausea | 22 (19.6) | 2 (1.8) |
| Neutropenia | 21 (18.8) | 15 (13.4) |
| Leukopenia | 13 (11.6) | 9 (8.0) |
| Febrile neutropenia | 9 (8.0) | 10 (8.9) |
| Patients who discontinued study | | |
| treatment, n (%) | | |
| Discontinuation due to treatment- | 15 (13.4)^{e} | |
| related AEs | | |

AE, adverse event; PPE, palmar-plantar erythrodysesthesia.
^{a}AEs by preferred term are listed if events of any grade occurred in ≥30% of patients in the erdafitinib group or if events of grade 3-4 occurred in ≥5% of patients.
^{b}Most frequent treatment-related AEs leading to discontinuation of erdafitinib included eye disorders (3 patients) and skin and subcutaneous disorders (3 patients).
^{c}Treatment-related AE leading to death was reported as sudden death.
^{d}AEs by preferred term are listed if events of any grade occurred in ≥20% of patients in the chemotherapy group or if events of grade 3-4 occurred in ≥5% of patients.
^{e}Most frequent treatment-related AEs leading to discontinuation of chemotherapy included blood and lymphatic system disorders (5 patients) and infections and infestations (3 patients).
^{f}Treatment-related AEs leading to death in the chemotherapy arm included febrile bone marrow aplasia (2 patients), febrile neutropenia (1 patient), septic shock (2 patients), and atypical pneumonia (1 patient)

TABLE 15
Safety results

| Patients with | Erdafitinib | | Chemotherapy | |
|---|---|---|---|---|
| AEs of | (n = 135) | | (n = 112) | |
| interest, n (%) | Any grade | Grade 3-4 | Any grade | Grade 3-4 |
| Nail disorders^{a} | 90 (66.7) | 15 (11.1) | 6 (5.4) | 0 |
| Skin disorders^{b} | 74 (54.8) | 16 (11.9) | 14 (12.5) | 0 |
| Eye disorders | 57 (42.2) | 3 (2.2) | 6 (5.4) | 0 |
| (excluding | | | | |
| central serous | | | | |
| retinopathy)^{c} | | | | |
| Central serous | 23 (17.0) | 3 (2.2) | 0 | 0 |
| retinopathy^{d} | | | | |

^{a}Nail disorders: nail bed bleeding, nail discoloration, nail disorder, nail dystrophy, nail ridging, nail toxicity, onychalgia, onychoclasis, onycholysis, paronychia, onychomadesis.
^{b}Skin disorders: blister, dry skin, erythema, hyperkeratosis, palmar erythema, palmar-plantar erythrodysesthesia syndrome, plantar erythema, rash, rash erythematous, rash generalized, rash macular, rash maculo-papular, skin atrophy, skin exfoliation, skin fissures, skin lesion, skin ulcer, toxic skin eruption, xeroderma.
^{c}Eye disorders (excluding central serous retinopathy): blepharitis, cataract, cataract subcapsular, conjunctival hemorrhage, conjunctival hyperemia, conjunctival irritation, corneal erosion, corneal infiltrates, dry eye, eye inflammation, eye irritation, eye pain, foreign body sensation in eyes, keratitis, lacrimation increased, night blindness, ocular hyperemia, photophobia, vision blurred, visual acuity reduced, visual impairment, xanthopsia, xerophthalmia, chorioretinitis, conjunctivitis, ulcerative keratitis.
^{d}Central serous retinopathy: retinal detachment, vitreous detachment, retinal edema, retinopathy, chorioretinopathy, detachment of retinal pigment epithelium, detachment of macular retinal pigment epithelium, macular detachment, serous retinal detachment, subretinal fluid, retinal thickening, chorioretinitis, serous retinopathy, maculopathy, choroidal effusion

### Conclusion

The results presented above demonstrate that the study's primary objective for Cohort 1 was met: Erdafitinib significantly prolonged or extended OS compared to chemotherapy in patients with advanced/mUC with FGFR alterations after prior treatment with anti-PD-(L)1 (i.e., median OS of 12.1 vs 7.8 months; HR=0.64; p-value=0.0050). Erdafitinib provided a 36% reduction in risk of death compared to chemotherapy. The OS benefit of erdafitinib was consistent across clinically relevant subgroups. Erdafitinib also significantly prolonged PFS (i.e., median PFS of 5.5 vs 2.7 months; HR=0.58; p-value=0.0002). ORR was also significantly greater for Erdafitinib, with similar DOR. The treatment arms were balanced in demographic and baseline disease characteristics. The safety results were consistent with the known safety profiles for Erdafitinib and chemotherapy in this patient population.

The phase 3 study supports the clinical efficacy of erdafitinib as the standard of care option for mUC patients with FGFR alterations after anti-PD-(L)1 treatment.

### Results Part 2

### Patients

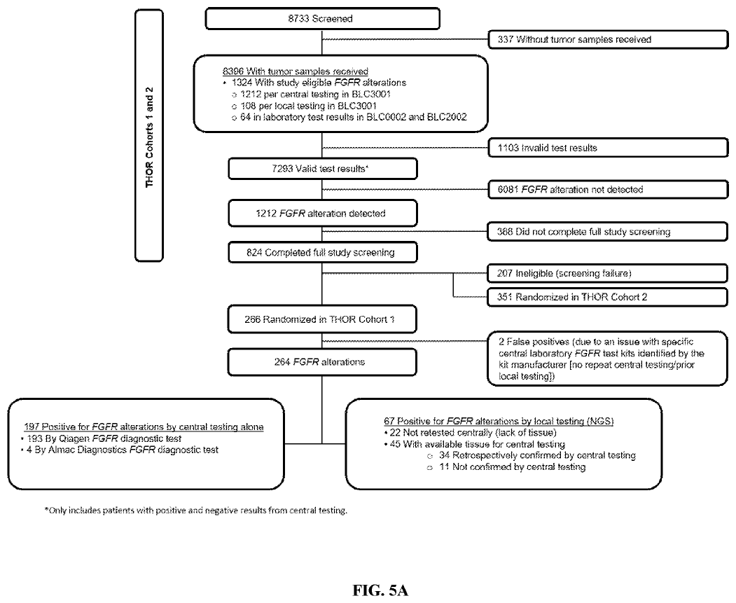

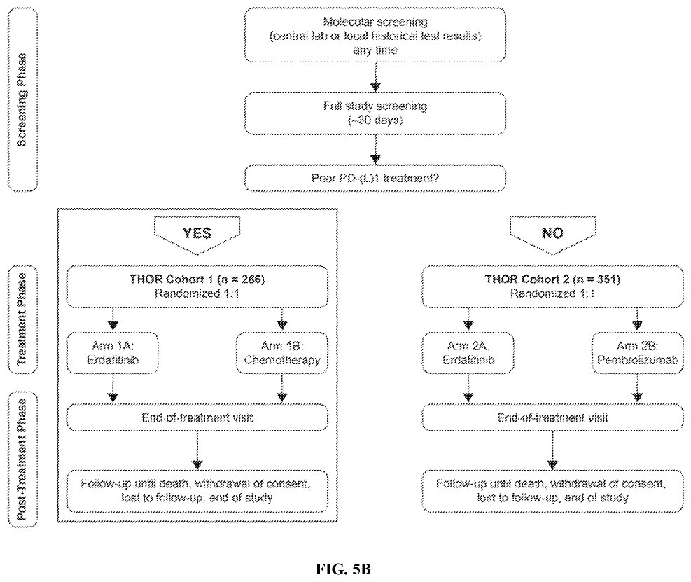

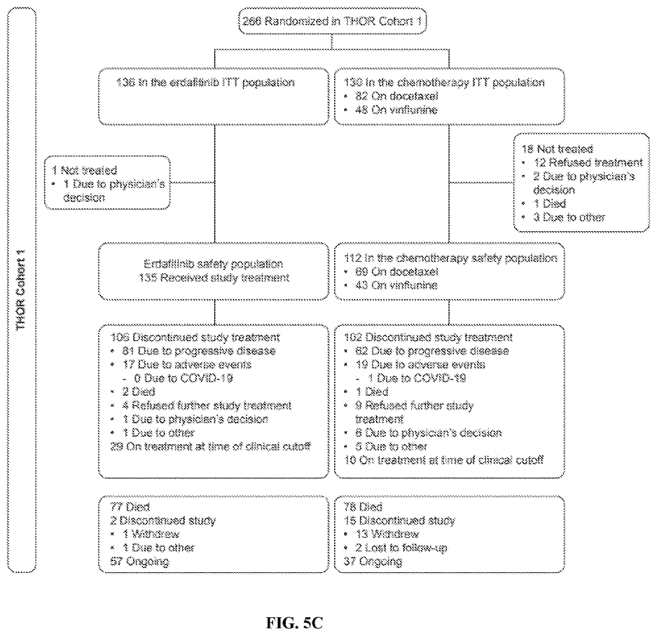

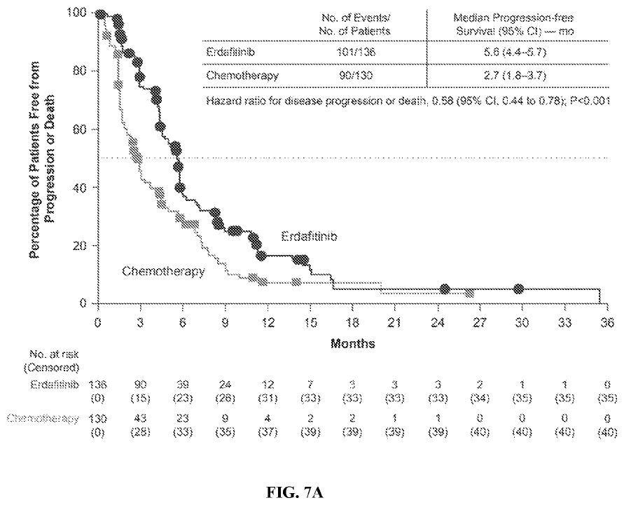

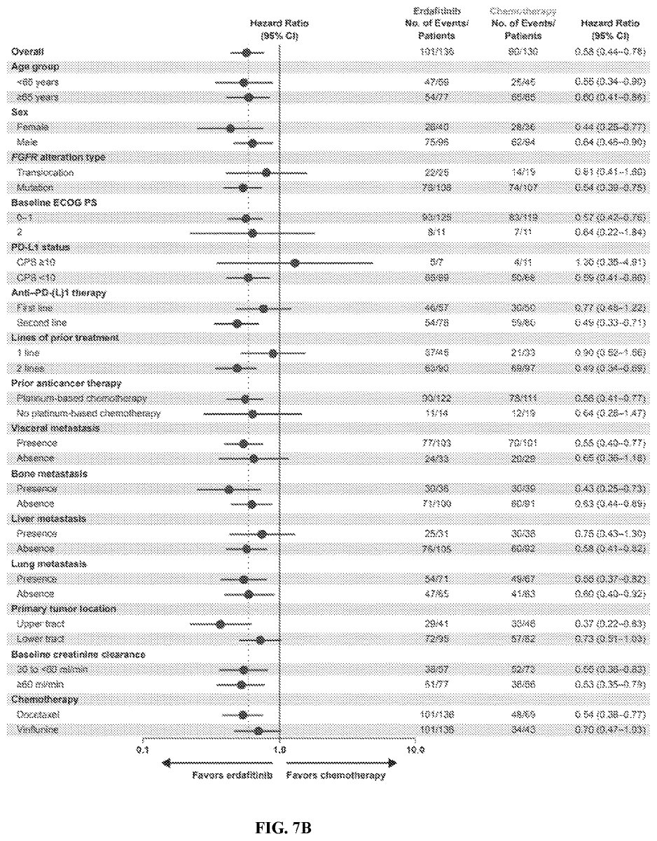

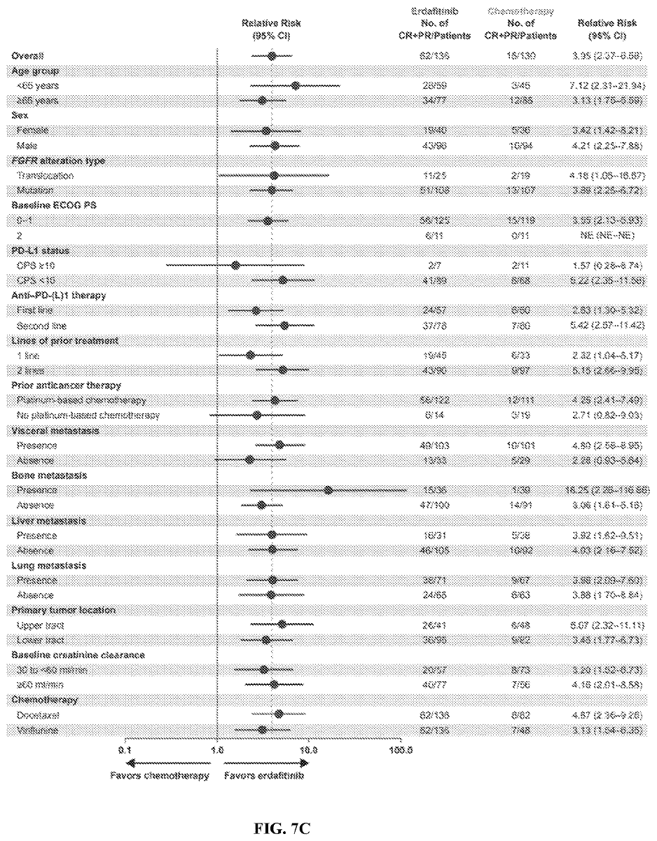

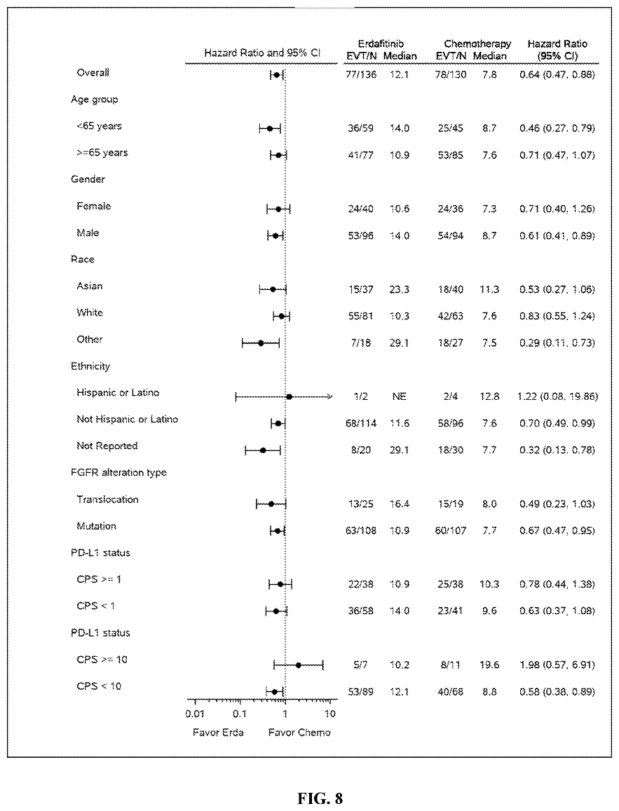

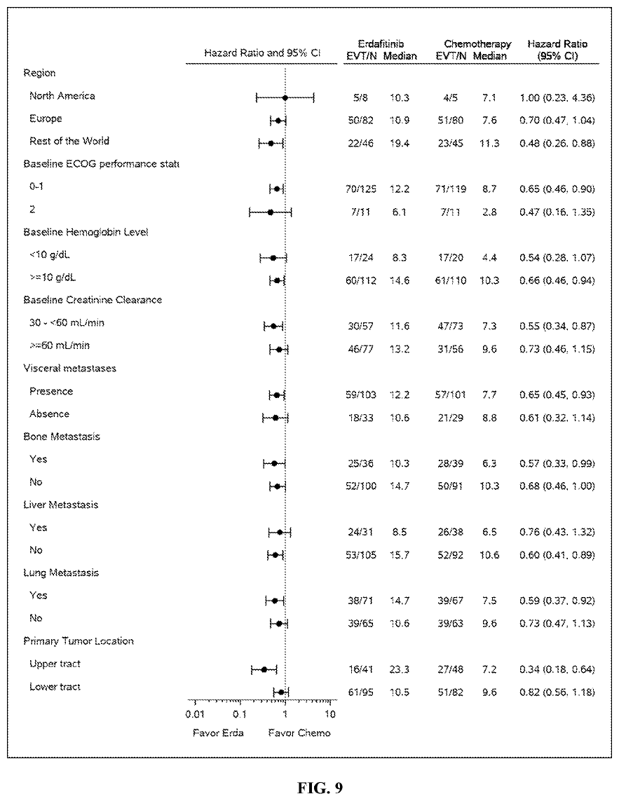

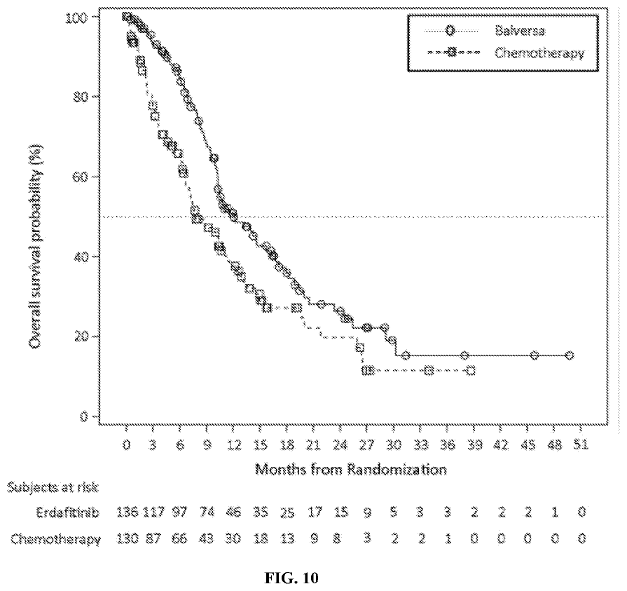

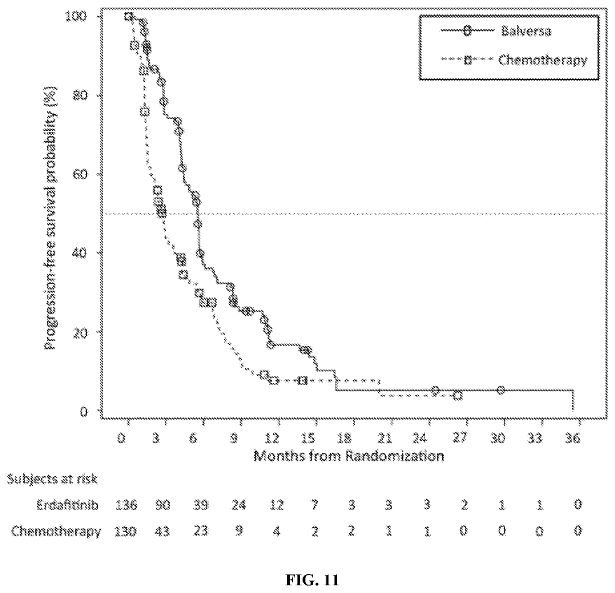

Of a total of 8733 patients centrally screened for molecular eligibility in the THOR trial (Cohorts 1 and 2), 8396 had tumor samples available with any test results, 7293 had valid central laboratory test results; of patients with validated central test results, 1212 had FGFR alterations (positivity rate, 16.6%; FIGS. 5A and 5B). A total of 1324 patients with any test results had FGFR alterations detected, with 1212 based on central laboratory test results, 108 on local laboratory test results (patients with local results may also have had central results), and 64 transferred from other studies (ANNAR [NCT03955913] and NORSE [NCT03473743]). In Cohort 1, 266 patients were randomized, 136 to the erdafitinib group and 130 to the chemotherapy group (FIG. 5C). An imbalance was observed in those not treated between groups (one in the erdafitinib group and 18 in the chemotherapy group), largely due to 12 patients refusing treatment in the chemotherapy group. 99.2% of patients in Cohort 1 had FGFR alterations (two patients had FGFR alterations on central testing that were later identified as false positives after randomization due to an issue with specific central laboratory FGFR test kits identified by the kit manufacturer; these two patients did not have repeat central testing nor prior local testing). In Cohort 1, 197 of 264 patients (74.2%) who had FGFR alterations were enrolled based on central test results; 67 patients were enrolled by local tests (tissue, n=60; blood, n=6; unspecified, n=1). 80.8% had FGFR mutations, 16.5% had FGFR fusions, and 1.9% had both FGFR mutations and fusions (FIGS. 5D, 5E, 7B, and 7C and Table 17). No patients had FGFR2 alterations; the FGFR3 S249C mutation was the most prevalent FGFR alteration (46.6%), followed by the FGFR3 Y373C mutation (16.9%) and the FGFR3-TACC3_V1 fusion (9.8%). The demographic and clinical characteristics of the patients at baseline were balanced across the erdafitinib and chemotherapy treatment groups (Tables 16 and 18). Only one patient was identified as Black; this was primary due to low enrollment in the United States and restrictions on reporting of race per local regulations on clinical practice (eg, France). Most patients (89.7%) with PD-L1 results had low PD-L1 expression (combined positive score <10 [Dako PD-L1 IHC 22C3 assay, Labcorp]), with baseline PD-L1 expression not reported for some patients due to insufficient tumor availability.

All patients had prior treatment with an anti-PD-(L)1 therapy except three patients incorrectly assigned (Table 19). Over half of patients in both treatment groups received an anti-PD-(L)1 as a single agent in the second-line setting (erdafitinib, 55.9%; chemotherapy, 58.5%). One-third (33.1%) of patients in the erdafitinib group and one-quarter (25.4%) of patients in the chemotherapy group received one line of prior systemic therapy. Although not required by the study protocol, the majority of patients (89.1%) received at least one line of prior chemotherapy (50.8% had prior cisplatin; 29.3% had prior carboplatin).

### Efficacy

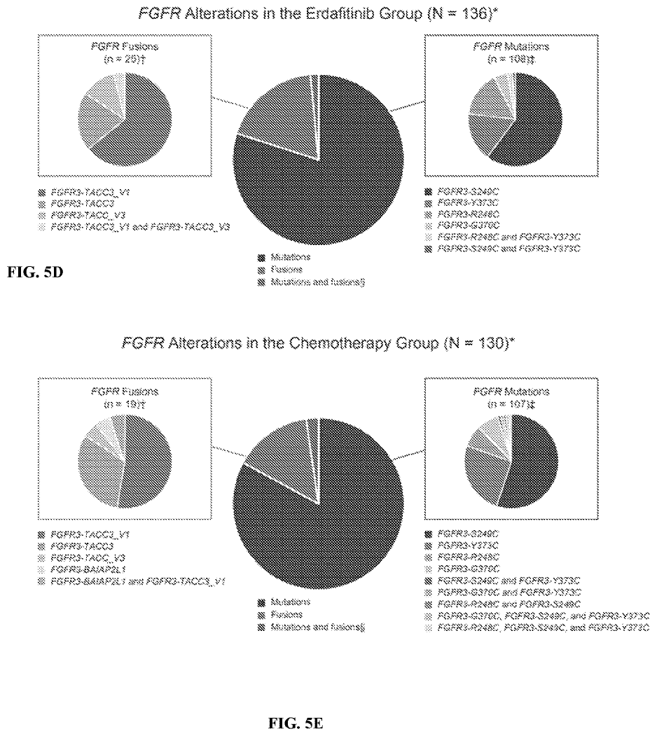

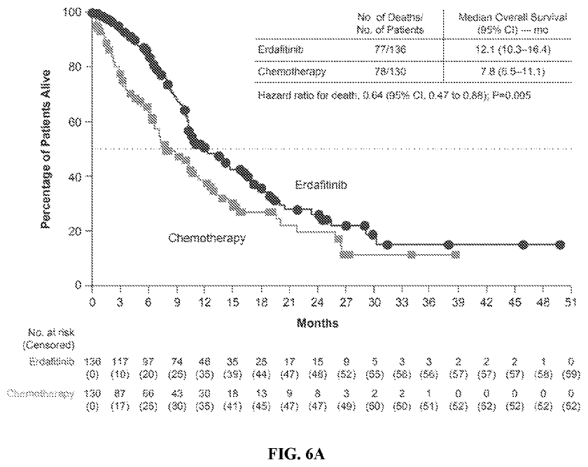

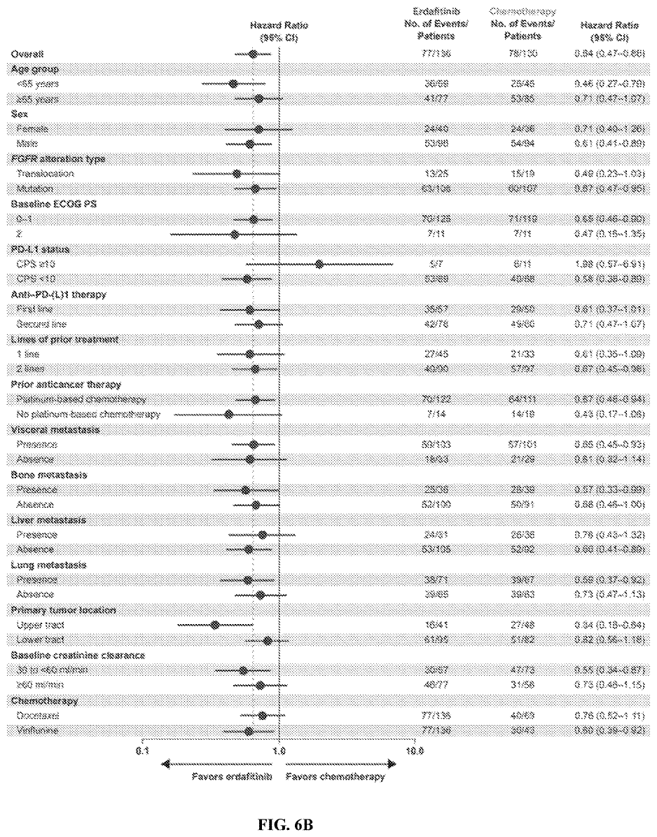

The median survival follow-up was 15.9 months (18.0 and 14.9 months in the erdafitinib and chemotherapy groups, respectively). At the interim analysis, a total of 155 deaths (~75% information fraction; 2-sided alpha of 0.019) had occurred (77 and 78 in the erdafitinib and chemotherapy groups, respectively). The median overall survival was 12.1 months in the erdafitinib group (95% confidence interval [CI], 10.3 to 16.4) and 7.8 months in the chemotherapy group (95% CI, 6.5 to 11.1), with an estimated HR of 0.64 (95% CI, 0.47 to 0.88; P=0.005). (FIG. 6A). The estimated percentage of patients alive at 6 and 12 months was 85% (95% CI, 77 to 90) and 51% (95% CI, 41 to 60) in the erdafitinib group versus 66% (95% CI, 56 to 74) and 38% (95% CI, 28 to 47) in the chemotherapy group, respectively. The effect of erdafitinib was generally consistent across subgroups (FIG. 6B). Following the interim analysis, the independent data monitoring committee recommended to stop the study, unblind data, and allow crossover from chemotherapy to erdafitinib.

The median progression-free survival was 5.6 months (95% CI, 4.4 to 5.7) and 2.7 months (95% CI, 1.8 to 3.7) in the erdafitinib and chemotherapy groups, respectively, with an estimated hazard ratio of 0.58 (95% CI, 0.44 to 0.78; P<0.001) (FIG. 7A). The progression-free survival and objective response rate differences were generally consistent in the subgroups evaluated (FIGS. 7B and 7C). The disease control rate was also higher in the erdafitinib group (82.4%) than in the chemotherapy group (43.1%) (RR, 1.9; 95% CI, 1.6 to 2.4). The confirmed objective response rate by investigator assessment (≥2 consecutive assessments) was 35.3% (27.3, 43.9) in the erdafitinib group and 8.5% (4.3, 14.6) in the chemotherapy group (Relative Risk (RR), 4.2; 95% CI, 2.3 to 7.6; p-value<0.001. p-value is estimated using Cochran-Haenszel (CMH) test with ECOG performance status (0 or 1 vs 2) as a stratification factor). The median duration of response was 4.9 months (95% CI, 3.8 to 7.5) in the erdafitinib group and 5.6 months (95% CI, 2.1 to 6.0) in the chemotherapy group.

Subsequent anticancer therapy was received by 92 (34.6%) patients, with 44 (32.4%) in the erdafitinib group and 48 (36.9%) in the chemotherapy group (Table 20).

### Safety

A total of 135 patients in the erdafitinib group and 112 patients in the chemotherapy group received at least one dose of study treatment. The median duration of exposure was longer with erdafitinib compared with chemotherapy (4.8 months [range, 0.2 to 38.2] vs. 1.4 months [range, 0.03 to 27.0]). In the erdafitinib group, 104 (77%) patients had dose up-titration from 8 to 9 mg, and 66 (48.9%) maintained ≥8 mg dose without dose reduction.

Adverse events of any cause occurred in 98.5% of patients in the erdafitinib group and 97.3% of patients in the chemotherapy group as shown in Table 21 (overall safety is shown in Table 23). Grade 3-4 treatment-related adverse events were 45.9% in the erdafitinib group and 46.4% in the chemotherapy group. The most common (≥5%) grade ≥3 treatment-related adverse events were palmar-plantar erythrodysesthesia syndrome (9.6%), stomatitis (8.1%), and onycholysis (5.9%) in the erdafitinib group and neutropenia (13.4%) and anemia (6.3%) in the chemotherapy group (Table 24).

Six (4.4%) and seven patients (6.3%) in the erdafitinib and chemotherapy groups, respectively, had treatment-emergent adverse events that led to death (Table 25). Fewer investigator-assessed treatment-related adverse events that led to death occurred in the erdafitinib group (0.7% [n=1]; sudden death [n=1]) than in the chemotherapy group (5.4% [n=6]; atypical pneumonia [n=1], febrile bone marrow aplasia [n=2], febrile neutropenia [n=1], septic shock [n=2]).

Treatment-related serious adverse events occurred in 18 (13.3%) and 27 patients (24.1%) in the erdafitinib and chemotherapy groups, respectively (Table 23; treatment-emergent serious adverse events in Table 26).

Adverse events of any cause led to treatment discontinuation in 19 (14.1%) and 20 (17.9%) patients in the erdafitinib and chemotherapy groups, respectively (Table 27). Fewer treatment-related adverse events led to treatment discontinuation in the erdafitinib group (8.1% vs. 13.4%).

Grade 3-4 adverse events of interest based on the known safety profile of erdafitinib included nail disorders (11.1%), skin disorders (11.9%), and central serous retinopathy (2.2%) (Table 28). In 16 of 23 patients (70%) with central serous retinopathy of any grade, events were resolved by the clinical cutoff date; of those with ongoing events, 5 of 7 (71%) were grade 1.

### Conclusion

8733 patients were screened for molecular eligibility in this study; 1212 of 7293 patients had FGFR alterations as assessed via central screening (16.6% positivity rate). 266 patients underwent randomization at the prespecified interim analysis; 136 to erdafitinib and 130 to chemotherapy. Median follow-up was 15.9 months. Overall survival was significantly longer with erdafitinib versus chemotherapy (median overall survival, 12.1 vs. 7.8 months; hazard ratio for death, 0.64; 95% confidence interval [CI], 0.47 to 0.88; P=0.005). Progression-free survival was also prolonged with erdafitinib (median progression-free survival, 5.6 vs. 2.7 months; hazard ratio for progression or death, 0.58; 95% CI, 0.44 to 0.78; P<0.001). The incidence of Grade 3-4 treatment-related adverse events was similar in the two groups (45.9% in the erdafitinib group and 46.4% in the chemotherapy group). Fewer treatment-related adverse events leading to death (0.7% vs. 5.4%) were reported with erdafitinib than chemotherapy.

In conclusion, erdafitinib significantly prolonged median overall survival compared with chemotherapy in patients with mUC and FGFR alt after prior anti-PD-(L)1 treatment, with a median overall survival of about 1 year (HR=0.64).

TABLE 16
Demographics and Disease Characteristics of the Patients at Baseline.

| | Erdafitinib | Chemotherapy |
|---|---|---|
| Characteristic | (N = 136) | (N = 130) |
| Median age (range)-yr | 66 (32-85) | 69 (35-86) |
| Age subgroup-no. (%) | | |
| <65 | 59 (43.4) | 45 (34.6) |
| ≥65 | 77 (56.6) | 85 (65.4) |
| Sex-no. (%) | | |
| Male | 96 (70.6) | 94 (72.3) |
| Female | 40 (29.4) | 36 (27.7) |
| Race-no. (%) | | |
| White | 81 (59.6) | 63 (48.5) |
| Asian | 37 (27.2) | 40 (30.8) |
| Black | 0 | 1 (0.8) |
| Multiple | 0 | 1 (0.8) |
| Not reported | 18 (13.2) | 25 (19.2) |
| Geographic region-no. (%) | | |
| North America | 8 (5.9) | 5 (3.8) |
| Europe | 82 (60.3) | 80 (61.5) |
| Rest of the World | 46 (33.8) | 45 (34.6) |
| Visceral metastasis-no. (%) | | |
| Present* | 101 (74.3) | 97 (74.6) |
| Absent | 35 (25.7) | 33 (25.4) |
| ECOG PS-no. (%)^{†} | | |
| 0 | 63 (46.3) | 51 (39.2) |
| 1 | 61 (44.9) | 66 (50.8) |
| 2 | 12 (8.8) | 13 (10.0) |
| Primary tumor location-no. (%) | | |
| Upper tract | 41 (30.1) | 48 (36.9) |
| Lower tract | 95 (69.9) | 82 (63.1) |
| PD-(L)1 status-no. (%)^{‡} | n = 96 | n = 79 |
| CPS <10 | 89 (92.7) | 68 (86.1) |
| CPS ≥10 | 7 (7.3) | 11 (13.9) |
| FGFR alterations-no. (%) | | |
| Mutations | 108 (79.4) | 107 (82.3) |
| Fusions | 25 (18.4) | 19 (14.6) |
| Mutations and fusions | 2 (1.5) | 3 (2.3) |
| Prior lines of systemic | | |
| therapy-no. (%) | | |
| 1 | 45 (33.1) | 33 (25.4) |
| 2 | 90 (66.2) | 97 (74.6) |
| 3 | 1 (0.7) | 0 |

*Visceral metastases in lung, liver, and bone.
^{†}Scores on the Eastern Cooperative Oncology Group (ECOG) scale range from 0 (no disability) to 5 (death).
^{‡}Based on patients with available data.

TABLE 17
Summary of FGFR Alterations.

| | Erdafitinib | Chemotherapy |
|---|---|---|
| FGFR alterations-no. (%) | (N = 136) | (N = 130) |
| Any FGFR alterations* | 135 (99.3) | 129 (99.2) |
| FGFR mutations (excluding fusions) | 108 (79.4) | 107 (82.3) |
| FGFR3 S249C | 65 (47.8) | 59 (45.4) |
| FGFR3 Y373C | 18 (13.2) | 27 (20.8) |
| FGFR3 R248C | 17 (12.5) | 8 (6.2) |
| FGFR3 G370C | 5 (3.7) | 8 (6.2) |
| FGFR3 R248C and FGFR3 Y373C | 2 (1.5) | 0 |
| FGFR3 S249C and FGFR3 Y373C | 1 (0.7) | 1 (0.8) |
| FGFR3 G370C and FGFR3 Y373C | 0 | 1 (0.8) |
| FGFR3 R248C and FGFR3 S249C | 0 | 1 (0.8) |
| FGFR3 G370C and FGFR3 S249C and | 0 | 1 (0.8) |
| FGFR3 Y373C | | |
| FGFR3 R248C and FGFR3 S249C and | 0 | 1 (0.8) |
| FGFR3 Y373C | | |
| FGFR fusions (excluding mutations) | 25 (18.4) | 19 (14.6) |
| FGFR3-TACC3_V1 | 16 (11.8) | 10 (7.7) |
| FGFR3-TACC3 | 5 (3.7) | 6 (4.6) |
| FGFR3-TACC3_V3 | 3 (2.2) | 1 (0.8) |
| FGFR-BAIAP2L1 | 0 | 1 (0.8) |
| FGFR-BAIAP2L1 and FGFR3-TACC3_V1 | 0 | 1 (0.8) |
| FGFR3-TACC3_V1 and FGFR3-TACC3_V3 | 1 (0.7) | 0 |
| FGFR mutations and fusions | 2 (1.5) | 3 (2.3) |
| FGFR3 S249C and FGFR3-TACC3_V1 | 2 (1.5) | 0 |
| FGFR3 R248C and FGFR3-TACC3_V1 | 0 | 1 (0.8) |
| FGFR3 S249C and FGFR3-TACC3_V3 | 0 | 1 (0.8) |
| FGFR3 Y373C and FGFR3-TACC3_V1 | 0 | 1 (0.8) |

*Two patients were randomized based on central laboratory results that were later identified as being false-positive results. FGFR results are based on central laboratory test results, local laboratory test results (patients with local results may also have had central results), and transferred from other studies (ANNAR [NCT03955913] and NORSE [NCT03473743]). Local tests used were next generation sequencing (NGS), direct digital counting methods, or the Qiagen Therascreen FGFR Rotor-Gene Q (RGQ) reverse transcription polymerase chain reaction (RT-PCR) test

TABLE 18
Additional Disease Characteristics of the Patients at Baseline.

| | Erdafitinib | Chemotherapy |
|---|---|---|
| Characteristic | (N = 136) | (N = 130) |
| Median time from | 27.3 (6.4 to 307.5) | 22.2 (1.9 to 342.3) |
| initial diagnosis | | |
| to first dose (range)-mo. | | |
| Median time from | 1.5 (−4.7 to 41.2) | 1.3 (0.4 to 5.5) |
| progression/relapse on last line | | |
| of prior chemotherapy | | |
| to first dose-mo. | | |
| Primary tumor location-no. (%) | | |
| Upper tract | 41 (30.1) | 48 (36.9) |
| Renal pelvis | 19 (14.0) | 20 (15.4) |
| Ureter | 22 (16.2) | 28 (21.5) |
| Lower tract | 95 (69.9) | 82 (63.1) |
| Bladder | 90 (66.2) | 74 (56.9) |
| Urethra | 5 (3.7) | 8 (6.2) |
| Prostate | 0 | 0 |
| Type of histology-no. (%) | | |
| Transitional cell carcinoma | 128 (94.1) | 124 (95.4) |
| Transitional cell | 8 (5.9) | 6 (4.6) |
| carcinoma with minor | | |
| components (<50%) overall | | |
| Tumor at initial | | |
| diagnosis-no. (%) | n = 136 | n = 129* |
| TX | 3 (2.2) | 10 (7.8) |
| T0 | 0 | 1 (0.8) |
| Ta | 13 (9.6) | 17 (13.2) |
| Tis | 0 | 0 |
| T1 | 25 (18.4) | 26 (20.2) |
| T2 | 45 (33.1) | 23 (17.8) |
| T3 | 37 (27.2) | 32 (24.8) |
| T4 | 10 (7.4) | 17 (13.2) |
| Lymph node at initial | | |
| diagnosis-no. (%) | n = 136 | n = 129* |
| NX | 38 (27.9) | 32 (24.8) |
| N0 | 63 (46.3) | 62 (48.1) |
| N1 | 18 (13.2) | 8 (6.2) |
| N2 | 11 (8.1) | 19 (14.7) |
| N3 | 6 (4.4) | 8 (6.2) |
| Metastatic at initial | | |
| diagnosis-no. (%) | n = 133 | n = 129* |
| M0 | 105 (78.9) | 96 (74.4) |
| M1 | 28 (21.1) | 33 (25.6) |
| Visceral metastasis-no. (%) | | |
| Present | 101 (74.3) | 97 (74.6) |
| Lung | 71 (52.2) | 67 (51.5) |
| Liver | 31 (22.8) | 38 (29.2) |
| Bone | 36 (26.5) | 39 (30.0) |
| Absent (lung, liver, or bone) | 35 (25.7) | 33 (25.4) |

*TNM classification was not available for one patient in the chemotherapy group.

TABLE 19
Prior Systemic Therapy.

| | Erdafitinib | Chemotherapy |
|---|---|---|
| Patients receiving prior therapy-no. (%)* | (N = 136)^{†} | (N = 130) |
| One line of prior systemic therapy | 45 (33.1) | 33 (25.4) |
| Chemotherapy + anti-PD-(L)1^{‡} | 33 (24.3) | 15 (11.5) |
| Anti-PD-(L)1^{§} | 11 (8.1) | 16 (12.3) |
| Chemotherapy | 1 (0.7) | 2 (1.5) |
| Two lines of prior systemic therapy | 90 (66.2) | 97 (74.6) |
| First line of therapy | | |
| Chemotherapy | 77 (56.6) | 76 (58.5) |
| Chemotherapy + anti-PD-(L)1 | 6 (4.4) | 10 (7.7) |
| Other | 7 (5.1) | 11 (8.5) |
| Second line of therapy | | |
| Anti-PD-(L)1 | 76 (55.9) | 76 (58.5) |
| Chemotherapy | 10 (7.4) | 14 (10.8) |
| Other | 4 (2.9) | 7 (5.4) |

*All enrolled patients received a prior anti-PD-(L)1 except for three patients as they were incorrectly assigned to Cohort 1 due to incorrect assignment flow in the interactive web response system at the time of randomization (erdafitinib group, n = 1; chemotherapy group, n = 2).
^{†}One patient in the erdafitinib group had three prior lines of systemic therapy.
^{‡}Includes patients who received other therapy in addition to chemotherapy + anti-PD-(L)1.
^{§}Includes patients who received other therapy in addition to anti-PD-(L)1.
PD-1, programmed cell death protein 1; PD-L1, programmed death-ligand 1.

TABLE 20
Subsequent Anticancer Therapy.

| Patients receiving subsequent | Erdafitinib | Chemotherapy |
|---|---|---|
| anticancer therapy-no. (%) | (N = 136) | (N = 130) |
| Any subsequent therapy | 44 (32.4) | 48 (36.9) |
| Number of subsequent therapy lines | | |
| 1 | 33 (24.3) | 40 (30.8) |
| 2 | 9 (6.6) | 8 (6.2) |
| 3 | 0 | 0 |
| >3 | 2 (1.5) | 0 |
| Chemotherapy | 21 (15.4) | 21 (16.2) |
| Carboplatin | 8 (5.9) | 9 (6.9) |
| Gemcitabine | 6 (4.4) | 10 (7.7) |
| Paclitaxel | 8 (5.9) | 6 (4.6) |
| Docetaxel | 4 (2.9) | 2 (1.5) |
| Vinflunine | 4 (2.9) | 0 |
| Cisplatin | 0 | 2 (1.5) |
| Methotrexate | 0 | 2 (1.5) |
| Pemetrexed | 2 (1.5) | 0 |
| Doxorubicin | 0 | 1 (0.8) |
| Vinblastine | 0 | 1 (0.8) |
| Immunotherapy | 9 (6.6) | 9 (6.9) |
| Pembrolizumab | 4 (2.9) | 4 (3.1) |
| Atezolizumab | 2 (1.5) | 2 (1.5) |
| Tislelizumab | 1 (0.7) | 2 (1.5) |
| Nivolumab | 1 (0.7) | 1 (0.8) |
| Avelumab | 1 (0.7) | 0 |
| FGFR inhibitors | 3 (2.2) | 10 (7.7) |
| Erdafitinib | 0 | 6 (4.6) |
| Darazantinib | 3 (2.2) | 2 (1.5) |
| Pemigatinib | 0 | 2 (1.5) |
| Antibody drug conjugate | 22 (16.2) | 14 (10.8) |
| Enfortumab vedotin | 19 (14.0) | 13 (10.0) |
| Sacituzumab govitecan | 3 (2.2) | 0 |
| Disitamab vedotin | 0 | 1 (0.8) |
| Other systemic therapy | 2 (1.5) | 1 (0.8) |
| Sitravatinib | 1 (0.7) | 1 (0.8) |
| Lenvatinib | 1 (0.7) | 0 |
| Investigational systemic therapy | 0 | 3 (2.3) |

FGFR, fibroblast growth factor receptor.

TABLE 21
Treatment-Emergent Adverse Events in the Safety Population.*

| | Erdafitinib | | | | Chemotherapy | | | |
|---|---|---|---|---|---|---|---|---|
| | (N = 135) | | | | (N = 112) | | | |
| Adverse event- | Any | | | Grade | Any | | | Grade |
| no. (%) | grade | Grade 1 | Grade 2 | ≥3 | grade | Grade 1 | Grade 2 | ≥3 |
| Hyperphosphatemia | 108 | 70 (51.9) | 31 (23.0) | 7 (5.2) | 0 | 0 | 0 | 0 |
| | (80.0) | | | | | | | |
| Diarrhea | 84 | 49 (36.3) | 31 (23.0) | 4 (3.0) | 19 | 7(6.3) | 9 (8.0) | 3 (2.7) |
| | (62.2) | | | | (17.0) | | | |
| Stomatitis | 65 | 22 (16.3) | 32 (23.7) | 11 (8.1) | 14 | 4 (3.6) | 8 (7.1) | 2 (1.8) |
| | (48.1) | | | | (12.5) | | | |
| Dry mouth | 53 | 45 (33.3) | 8 (5.9) | 0 | 4 | 4 (3.6) | 0 | 0 |
| | (39.3) | | | | (3.6) | | | |
| Palmar-plantar | 41 | 6 (4.4) | 22 (16.3) | 13 (9.6) | 1 | 0 | 1 (0.9) | 0 |
| erythrodysesthesia | (30.4) | | | | (0.9) | | | |
| syndrome | | | | | | | | |
| Dysgeusia | 37 | 28 (20.7) | 8 (5.9) | 1 (0.7) | 8 | 5 (4.5) | 3 (2.7) | 0 |
| | (27.4) | | | | (7.1) | | | |
| Alanine | 37 | 28 (20.7) | 8 (5.9) | 4 (3.0) | 4 | 2 (1.8) | 1 (0.9) | 1 (0.9) |
| aminotransferase | (27.4) | | | | (3.6) | | | |
| increased | | | | | | | | |
| Constipation | 36 | 24 (17.8) | 12 (8.9) | 0 | 31 | 13 (11.6) | 16 (14.3) | 2 (1.8) |
| | (26.7) | | | | (27.7) | | | |
| Decreased appetite | 36 | 18 (13.3) | 14 (10.4) | 4 (3.0) | 23 | 10 (8.9) | 10 (8.9) | 3 (2.7) |
| | (26.7) | | | | (20.5) | | | |
| Anemia | 35 | 10 (7.4) | 15 | 10 (7.4) | 36 | 8 (7.1) | 19 (17.0) | 9 (8.0) |
| | (25.9) | | (11.1) | | (32.1) | | | |
| Alopecia | 34 | 29 (21.5) | 4 (3.0) | 1 (0.7) | 27 | 16 (14.3) | 11 (9.8) | 0 |
| | (25.2) | | | | (24.1) | | | |
| Dry skin | 31 | 23 (17.0) | 6 (4.4) | 2 (1.5) | 5 | 4 (3.6) | 1 (0.9) | 0 |
| | (23.0) | | | | (4.5) | | | |
| Onycholysis | 31 | 9 (6.7) | 14 (10.4) | 8 (5.9) | 1 | 0 | 1 (0.9) | 0 |
| | (23.0) | | | | (0.9) | | | |
| Weight decreased | 30 | 12 (8.9) | 15 (11.1) | 3 (2.2) | 3 | 3 (2.7) | 0 | 0 |
| | (22.2) | | | | (2.7) | | | |
| Aspartate | 29 | 21 (15.6) | 5 (3.7) | 3 (2.2) | 3 | 2 (1.8) | 1 (0.9) | 0 |
| aminotransferase | (21.5) | | | | (2.7) | | | |
| increased | | | | | | | | |
| Onychomadesis | 28 | 9 (6.7) | 17 (12.6) | 2 (1.5) | 2 | 1 (0.9) | 1 (0.9) | 0 |
| | (20.7) | | | | (1.8) | | | |
| Nail discoloration | 24 | 16 (11.9) | 7 (5.2) | 1 (0.7) | 2 | 1 (0.9) | 1 (0.9) | 0 |
| | (17.8) | | | | (1.8) | | | |
| Dry eye | 23 | 20 (14.8) | 3 (2.2) | 0 | 2 | 1 (0.9) | 1 (0.9) | 0 |
| | (17.0) | | | | (1.8) | | | |
| Asthenia | 20 | 6 (4.4) | 12 (8.9) | 2 (1.5) | 28 | 9 (8.0) | 15 (13.4) | 4 (3.6) |
| | (14.8) | | | | (25.0) | | | |
| Nausea | 20 | 10 (7.4) | 8 (5.9) | 2 (1.5) | 27 | 15 (13.4) | 10 (8.9) | 2 (1.8) |
| | (14.8) | | | | (24.1) | | | |
| Neutropenia | 0 | 0 | 0 | 0 | 22 | 1 (0.9) | 5 (4.5) | 16 (14.3) |
| | | | | | (19.6) | | | |
| Fatigue | 20 | 12 (8.9) | 8 (5.9) | | 21 | 13 | | 4 (3.6) |
| | (14.8) | | | 0 | (18.8) | (11.6) | 4 (3.6) | |

*Listed are treatment-emergent adverse events of any cause by preferred term and worst toxicity grade that were reported in more than 15% of the patients in either treatment group.

TABLE 22
Representativeness of Study Participants

| | |
|---|---|
| Disease under | |
| investigation | Advanced or metastatic urothelial cancer. |

Special considerations related to:

| | |
|---|---|
| Sex and | Worldwide, bladder cancer (urothelial |
| gender | cancer accounts for 90% of |
| | bladder cancers worldwide) is |
| | approximately four times more |
| | common in men compared with |
| | women (incidence: 9.6 vs. |
| | 2.4/100,000, respectively).¹ Mortality |
| | rates are, similarly, four times |
| | greater in men versus women |
| | (3.2 vs. 0.9/100,000, respectively). |
| | Gender disparity is thought to be |
| | primarily attributable to gender |
| | differences in smoking tobacco, |
| | but may also involve occupational |
| | chemical exposure, alcohol, and |
| | red meat consumption.¹ |
| Age | In the United States, the median |
| | average age of diagnosis is 73 years |
| | of age; 90% of new bladder cancer |
| | cases are in people ≥55 years of |
| | age.² |
| Race or | The SEER 2020 urinary bladder |
| ethnic group | cancer (invasive & in situ) age- |
| | adjusted incidence rate (2020) |
| | (all stages at diagnosis) for all races or |
| | ethnic groups is: 17.3/100,000 |
| | (95% CI, 17.1 to 17.5). Data for |
| | specific race or ethnic groups are as follows: |
| | Hispanic (any race): 9.2/100,000 |
| | (95% CI, 8.8 to 9.6) |
| | Non-Hispanic American Indian/Alaska |
| | Native: 11.8/100,000 (95% CI, 8.9 to 15.2) |
| | Non-Hispanic Asian/Pacific Islander: |
| | 8.0/100,000 (95% CI, 7.6 to 8.5) |
| | Non-Hispanic Black: 11.1/100,000 |
| | (95% CI, 10.6 to 11.6) |
| | Non-Hispanic White: 21.1/100,000 |
| | (95% CI, 20.8 to 21.4) |
| | Black (includes Hispanic): 10.7/100,000 |
| | (95% CI, 10.2 to 11.2) |
| | White (includes Hispanic): 19.1/100,000 |
| | (95% CI, 18.8 to 19.3)³ |
| | The SEER 5-year urinary bladder |
| | cancer (invasive & in situ) survival |
| | rate (2013 to 2019) for distant |
| | metastases at diagnosis (any age) for |
| | all races or ethnic groups is 8.3% |
| | (95% CI, 7.5 to 9.2). Data for |
| | specific race or ethnic groups are as follows: |
| | Hispanic (any race): 9.1% (95% CI, 6.5 to 12.1) |
| | Non-Hispanic American Indian/Alaska Native: |
| | 20.2% (95% CI, 7.6 to 37.2) |
| | Non-Hispanic Asian/Pacific Islander: |
| | 10.9% (95% CI, 6.9 to 15.9) |
| | Non-Hispanic Black: 5.2% (95% CI, 3.4 to 7.6) |
| | Non-Hispanic White: 8.3% (95% CI, 7.4 to 9.3) |
| | Black (includes Hispanic): 5.2% |
| | (95% CI, 3.4 to 7.6) |
| | White (includes Hispanic): 8.4% |
| | (95% CI, 7.5 to 9.4)³ |
| Geography | Highest rates of bladder cancer |
| | are predominantly observed in |
| | Southern Europe, Western Europe, |
| | and North America. Regions with |
| | the lowest incidence rates include |
| | Middle Africa, Central America, |
| | and West Africa, possibly attributable |
| | to lower industrial chemical |
| | exposure and access to tobacco.¹ |
| | Mortality is greatest in North and |
| | East Africa and the Middle East, |
| | with the highest mortality in Egypt |
| | (6.6/100,000). These relatively |
| | high mortality rates occur in regions |
| | where schistosomiasis infection |
| | leads to high rates of incidence.¹ |
| Overall | The THOR study Cohort 1 sex |
| representativeness | distribution of patients (male, 71%; |
| of | female, 29%) was representative |
| this trial | of the global population with |
| | bladder cancer. The majority of |
| | patients were either White (54%) or |
| | Asian (29%) and non-Hispanic or |
| | Latino (79%); however, in nearly |
| | 20% of patients, race and ethnicity |
| | were not reported. |
| | Only one patient was identified as |
| | Black; this was primarily due to |
| | low enrollment in the United States |
| | and restrictions on reporting of |
| | race per local regulations on the |
| | capture of this information in clinical |
| | studies (e.g., France). |
| | THOR was a worldwide study |
| | with patients in THOR Cohort 1 |
| | representative of regions where |
| | bladder cancer incidence is relatively |
| | high (Europe, 60%; North America, |
| | 6%; rest of the world, 34%). |
| | Patients in THOR were enrolled in |
| | sites from 23 countries/territories |
| | (Asia: Israel and Russia; Europe: |
| | Austria, Belgium, Bulgaria, France, |
| | Germany, Greece, Hungary, Italy, |
| | Netherlands, Spain, Turkey, and |
| | the United Kingdom; North America: |
| | Canada and the United States; |
| | South America: Argentina and Brazil; |
| | Asia Pacific: Australia, China, |
| | Japan, South Korea, and Taiwan). |

SEER, The Surveillance, Epidemiology, and End Results Program.

TABLE 23
Overall Safety Summary.

| | Erdafitinib | Chemotherapy |
|---|---|---|
| Adverse events-no. (%) | (N = 135) | (N = 112) |
| Any adverse events | 133 (98.5) | 109 (97.3) |
| Related adverse events | 131 (97.0) | 97 (86.6) |
| Grade 3-4 adverse events | 85 (63.0) | 72 (64.3) |
| Related grade 3-4 adverse events | 62 (45.9) | 52 (46.4) |
| Serious adverse events | 56 (41.5) | 47 (42.0) |
| Related serious adverse events | 18 (13.3) | 27 (24.1) |
| Grade 3-4 serious adverse events | 52 (38.5) | 41 (36.6) |
| Related grade 3-4 serious adverse events | 16 (11.9) | 23 (20.5) |
| Adverse events leading to treatment discontinuation | 19 (14.1) | 20 (17.9) |
| Related adverse events leading to treatment | 11 (8.1) | 15 (13.4) |
| discontinuation | | |
| Adverse events leading to dose reduction | 93 (68.9) | 27 (24.1) |
| Related adverse events leading to dose reduction | 89 (65.9) | 24 (21.4) |
| Adverse events leading to dose interruption | 97 (71.9) | 35 (31.3) |
| Related adverse events leading to dose interruption | 89 (65.9) | 22 (19.6) |
| Adverse events leading to death | 6 (4.4) | 7 (6.3) |
| Related adverse events leading to death | 1 (0.7) | 6 (5.4) |
| COVID-19-related adverse events | 12 (8.9) | 5 (4.5) |
| COVID-19-related serious adverse events | 1 (0.7) | 0 |

TABLE 24
Treatment-Related Adverse Events in the Safety Population.*

| | Erdafitinib | | | | Chemotherapy | | | |
|---|---|---|---|---|---|---|---|---|
| | (N = 135) | | | | (N = 112) | | | |
| Adverse event-no. (%) | Any grade | Grade 1 | Grade 2 | Grade ≥3 | Any grade | Grade 1 | Grade 2 | Grade ≥3 |
| Hyperphosphatemia | 106 (78.5) | 70 (51.9) | 29 (21.5) | 7 (5.2) | 0 | 0 | 0 | 0 |
| Diarrhea | 74 (54.8) | 43 (31.9) | 27 (20.0) | 4 (3.0) | 12 (10.7) | 3 (2.7) | 6 (5.4) | 3 (2.7) |
| Stomatitis | 62 (45.9) | 20 (14.8) | 31 (23.0) | 11 (8.1) | 13 (11.6) | 4 (3.6) | 7 (6.3) | 2 (1.8) |
| Dry mouth | 52 (38.5) | 44 (32.6) | 8 (5.9) | 0 | 3 (2.7) | 3 (2.7) | 0 | 0 |
| Palmar-plantar erythrodysesthesia | 41 (30.4) | 6 (4.4) | 22 (16.3) | 13 (9.6) | 1 (0.9) | 0 | 1 (0.9) | 0 |
| syndrome | | | | | | | | |
| Dysgeusia | 34 (25.2) | 25 (18.5) | 8 (5.9) | 1 (0.7) | 7 (6.3) | 4 (3.6) | 3 (2.7) | 0 |
| Alopecia | 32 (23.7) | 27 (20.0) | 4 (3.0) | 1 (0.7) | 24 (21.4) | 15 (13.4) | 9 (8.0) | 0 |
| Onycholysis | 31 (23.0) | 9 (6.7) | 14 (10.4) | 8 (5.9) | 1 (0.9) | 0 | 1 (0.9) | 0 |
| Dry skin | 30 (22.2) | 22 (16.3) | 6 (4.4) | 2 (1.5) | 4 (3.6) | 3 (2.7) | 1 (0.9) | 0 |
| Alanine aminotransferase increased | 29 (21.5) | 18 (13.3) | 7 (5.2) | 4 (3.0) | 3 (2.7) | 2 (1.8) | 0 | 1 (0.9) |
| Decreased appetite | 28 (20.7) | 15 (11.1) | 10 (7.4) | 3 (2.2) | 20 (17.9) | 8 (7.1) | 9 (8.0) | 3 (2.7) |
| Onychomadesis | 27 (20.0) | 8 (5.9) | 17 (12.6) | 2 (1.5) | 2 (1.8) | 1 (0.9) | 1 (0.9) | 0 |
| Aspartate aminotransferase increased | 25 (18.5) | 18 (13.3) | 5 (3.7) | 2 (1.5) | 1 (0.9) | 1 (0.9) | 0 | 0 |
| Nail discoloration | 24 (17.8) | 16 (11.9) | 7 (5.2) | 1 (0.7) | 2 (1.8) | 1 (0.9) | 1 (0.9) | 0 |
| Dry eye | 22 (16.3) | 19 (14.1) | 3 (2.2) | 0 | 2 (1.8) | 1 (0.9) | 1 (0.9) | 0 |
| Anemia | 16 (11.9) | 8 (5.9) | 4 (3.0) | 4 (3.0) | 31 (27.7) | 6 (5.4) | 18 (16.1) | 7 (6.3) |
| Nausea | 14 (10.4) | 6 (4.4) | 7 (5.2) | 1 (0.7) | 22 (19.6) | 12 (10.7) | 8 (7.1) | 2 (1.8) |
| Asthenia | 11 (8.1) | 4 (3.0) | 6 (4.4) | 0 | 21 (18.8) | 8 (7.1) | 11 (9.8) | 2 (1.8) |
| Constipation | 12 (8.9) | 8 (5.9) | 4 (3.0) | 0 | 21 (18.8) | 9 (8.0) | 10 (8.9) | 2 (1.8) |
| Neutropenia | 0 | 0 | 0 | 0 | 21 (18.8) | 1 (0.9) | 5 (4.5) | 15 (13.4) |
| Fatigue | 18 (13.3) | 12 (8.9) | 6 (4.4) | 0 | 17 (15.2) | 10 (8.9) | 3 (2.7) | 4 (3.6) |

*Listed are all treatment-related adverse events by preferred term and worst toxicity grade that were reported in more than 15% of the patients in either treatment group.

TABLE 25
Adverse Events Leading to Death.

| | Erdafitinib | Chemotherapy |
|---|---|---|
| Adverse events-no. (%)* | (N = 135) | (N = 112) |
| Any patient with one or more | 6 (4.4) | 7 (6.3) |
| adverse events leading to death | | |
| Sudden death | 2 (1.5) | 0 |
| Cardiorespiratory arrest | 1 (0.7) | 0 |
| Pneumonia aspiration | 1 (0.7) | 0 |
| Pneumonia necrotizing | 1 (0.7) | 0 |
| Renal failure | 1 (0.7) | 0 |
| Febrile bone marrow aplasia | 0 | 2 (1.8) |
| Septic shock | 0 | 2 (1.8) |
| Atypical pneumonia | 0 | 1 (0.9) |
| Febrile neutropenia | 0 | 1 (0.9) |
| General physical health deterioration | 0 | 1 (0.9) |

TABLE 26
Serious Adverse Events in the Safety Population.*

| | Erdafitinib | | | | Chemotherapy | | | |
|---|---|---|---|---|---|---|---|---|
| Adverse event-no. | (N = 135) | | | | (N = 112) | | | |
| (%) | Any grade | Grade 1 | Grade 2 | Grade ≥3 | Any grade | Grade 1 | Grade 2 | Grade ≥3 |
| Urinary tract infection | 6 (4.4) | 0 | 0 | 6 (4.4) | 2 (1.8) | 0 | 0 | 2 (1.8) |
| Hematuria | 5 (3.7) | 0 | 2 (1.5) | 3 (2.2) | 1 (0.9) | 0 | 0 | 1 (0.9) |
| Acute kidney injury | 3 (2.2) | 0 | 0 | 3 (2.2) | 0 | 0 | 0 | 0 |
| Hyponatremia | 3 (2.2) | 0 | 0 | 3 (2.2) | 1 (0.9) | 0 | 0 | 1 (0.9) |

*Listed are all adverse events of any cause by preferred term and worse toxicity grade that were reported in more than 2% of the patients.

TABLE 27
Adverse Events Leading to Treatment Discontinuation.

| | Erdafitinib | Chemotherapy |
|---|---|---|
| Adverse events-no. (%)* | (N = 135) | (N = 112) |
| Any patient with one or more adverse events | 19 (14.1) | 20 (17.9) |
| leading to treatment discontinuation | | |
| General physical health deterioration | 2 (1.5) | 2 (1.8) |
| Onycholysis | 2 (1.5) | 0 |
| Sudden death | 2 (1.5) | 0 |
| Cardiorespiratory arrest | 1 (0.7) | 0 |
| COVID-19 | 1 (0.7) | 0 |
| Detachment of retinal pigment epithelium | 1 (0.7) | 0 |
| Dry skin | 1 (0.7) | 0 |
| Dysphagia | 1 (0.7) | 0 |
| Extremity necrosis | 1 (0.7) | 0 |
| Hematuria | 1 (0.7) | 0 |
| Hypotension | 1 (0.7) | 0 |
| Keratitis | 1 (0.7) | 0 |
| Mouth ulceration | 1 (0.7) | 0 |
| Nail discoloration | 1 (0.7) | 0 |
| Nail dystrophy | 1 (0.7) | 0 |
| Paronychia | 1 (0.7) | 0 |
| Pneumonia aspiration | 1 (0.7) | 0 |
| Pneumonia necrotizing | 1 (0.7) | 0 |
| Renal failure | 1 (0.7) | 0 |
| Urinary retention | 1 (0.7) | 0 |
| Vision blurred | 1 (0.7) | 0 |
| Anemia | 0 | 1 (0.9) |
| Atypical pneumonia | 0 | 1 (0.9) |
| Diarrhea | 0 | 2 (1.8) |
| Dyspnea | 0 | 1 (0.9) |
| Edema peripheral | 0 | 1 (0.9) |
| Febrile bone marrow aplasia | 0 | 3 (2.7) |
| Febrile neutropenia | 0 | 1 (0.9) |
| Fractured sacrum | 0 | 1 (0.9) |
| Hyponatremia | 0 | 1 (0.9) |
| Ileus | 0 | 1 (0.9) |
| Peripheral sensory neuropathy | 0 | 1 (0.9) |
| Pneumonitis | 0 | 1 (0.9) |
| Sciatica | 0 | 1 (0.9) |
| Septic shock | 0 | 2 (1.8) |
| Syncope | 0 | 1 (0.9) |
| Vomiting | 0 | 1 (0.9) |

TABLE 28
Adverse Events of Interest in the Safety Population.*

| | Erdafitinib | | | | Chemotherapy | | | |
|---|---|---|---|---|---|---|---|---|
| | (N = 135) | | | | (N = 112) | | | |
| Adverse event-no. (%) | Any grade | Grade 1 | Grade 2 | Grade ≥3 | Any grade | Grade 1 | Grade 2 | Grade ≥3 |
| Nail disorders^{†} | 90 (66.7) | 25 (18.5) | 50 (37.0) | 15 (11.1) | 6 (5.4) | 4 (3.6) | 2 (1.8) | 0 |
| Skin disorders^{‡} | 74 (54.8) | 27 (20) | 31 (23.0) | 16 (11.9) | 14 (12.5) | 12 (10.7) | 2 (1.8) | 0 |
| Eye disorders | 57 (42.2) | 37 (27.4) | 17 (12.6) | 3 (2.2) | 6 (5.4) | 2 (1.8) | 4 (3.6) | 0 |
| (excluding central | | | | | | | | |
| serous retinopathy)^{§} | | | | | | | | |
| Central serous retinopathy | 23 (17.0) | 12 (8.9) | 8 (5.9) | 3 (2.2) | 0 | 0 | 0 | 0 |
| Chorioretinopathy | 8 (5.9) | 5 (3.7) | 3 (2.2) | 0 | 0 | 0 | 0 | 0 |
| Detachment of retinal | 7 (5.2) | 3 (2.2) | 2 (1.5) | 2 (1.5) | 0 | 0 | 0 | 0 |
| pigment epithelium | | | | | | | | |
| Subretinal fluid | 5 (3.7) | 4 (3.0) | 1 (0.7) | 0 | 0 | 0 | 0 | 0 |
| Macular detachment | 2 (1.5) | 2 (1.5) | 0 | 0 | 0 | 0 | 0 | 0 |
| Retinopathy | 2 (1.5) | 0 | 2 (1.5) | 0 | 0 | 0 | 0 | 0 |
| Detachment of macular | 1 (0.7) | 0 | 0 | 1 (0.7) | 0 | 0 | 0 | 0 |
| retinal pigment epithelium | | | | | | | | |

*Listed are all adverse events of any cause by preferred term and worse toxicity grade that were reported in more than 2% of the patients.
^{†}Nail disorders: nail bed bleeding, nail discoloration, nail disorder, nail dystrophy, nail ridging, nail toxicity, onychalgia, onychoclasis, onycholysis, paronychia, onychomadesis.
^{‡}Skin disorders: blister, dry skin, erythema, hyperkeratosis, palmar erythema, palmar-plantar erythrodysesthesia syndrome, plantar erythema, rash, rash erythematous, rash generalized, rash macular, rash maculo-papular, skin atrophy, skin exfoliation, skin fissures, skin lesion, skin ulcer, toxic skin eruption, xeroderma.
^{§}Eye disorders (excluding central serous retinopathy): blepharitis, cataract, cataract subcapsular, conjunctival hemorrhage, conjunctival hyperemia, conjunctival irritation, corneal erosion, corneal infiltrates, dry eye, eye inflammation, eye irritation, eye pain, foreign body sensation in eyes, keratitis, lacrimation increased, night blindness, ocular hyperemia, photophobia, vision blurred, visual acuity reduced, visual impairment, xanthopsia, xerophthalmia, chorioretinitis, conjunctivitis, ulcerative keratitis.

### Example 2: FDA Approved Drug Product Label

The FDA approved the following drug product label on Jan. 19, 2024, for BALVERSA (erdafitinib), which is the reference listed drug for erdafitinib.

### BALVERSA—Erdafitinib Tablet, Film Coated

Janssen Products LP

### Highlights of Prescribing Information

These highlights do not include all the information needed to use BALVERSA safely and effectively. See full prescribing information for BALVERSA.

### BALVERSA® (Erdafitinib) Tablets, for Oral Use

Initial U.S. Approval: 2019

### Recent Major Changes

| | |
|---|---|
| Indications and Usage (1) | January 2024 |
| Dosage and Administration (2.2), (2.3) | January 2024 |
| Warnings and Precautions (5.1), (5.2) | January 2024 |

### Indications and Usage

BALVERSA is a kinase inhibitor indicated for the treatment of adult patients with locally advanced or metastatic urothelial carcinoma (mUC) with susceptible FGFR3 genetic alterations whose disease has progressed on or after at least one line of prior systemic therapy.
Select patients for therapy based on an FDA-approved companion diagnostic for BALVERSA. (1, 2.1)

### Limitations of Use

BALVERSA is not recommended for the treatment of patients who are eligible for and have not received prior PD-1 or PD-L1 inhibitor therapy. (1, 14.1)

### Dosage and Administration

Confirm the presence of FGFR3 genetic alterations in tumor specimens prior to initiation of treatment with BALVERSA. (2.1)
Recommended initial dosage: 8 mg orally once daily with a dose increase to 9 mg daily if criteria are met. (2.2)
Swallow whole with or without food. (2.2)

### Dosage Forms and Strengths

Tablets: 3 mg, 4 mg, and 5 mg. (3)

### Contraindications

None. (4)

### Warnings and Precautions

Ocular disorders: BALVERSA can cause central serous retinopathy/retinal pigment epithelial detachment (CSR/RPED). Perform monthly ophthalmological examinations during the first four months of treatment, every 3 months afterwards, and at any time for visual symptoms. Withhold BALVERSA when CSR/RPED occurs and permanently discontinue if it does not resolve within 4 weeks or if Grade 4 in severity. (2.3, 5.1)
Hyperphosphatemia: Increases in phosphate levels are a pharmacodynamic effect of BALVERSA. Monitor for hyperphosphatemia and manage with dose modifications when required. (2.3, 5.2)
Embryo-fetal toxicity: Can cause fetal harm. Advise patients of the potential risk to the fetus and to use effective contraception (5.3, 8.1, 8.3)

### Adverse Reactions

The most common (≥20%) adverse reactions, including laboratory abnormalities, were increased phosphate, nail disorders, stomatitis, diarrhea, increased creatinine, increased alkaline phosphatase, increased alanine aminotransferase, decreased hemoglobin, decreased sodium, increased aspartate aminotransferase, fatigue, dry mouth, dry skin, decreased phosphate, decreased appetite, dysgeusia, constipation, increased calcium, dry eye, palmar-plantar erythrodysesthesia syndrome, increased potassium, alopecia, and central serous retinopathy. (6.1)

To report SUSPECTED ADVERSE REACTIONS, contact Janssen Products, LP. at 1-800-526-7736 (1-800-JANSSEN and www.BALVERSA.com) or FDA at 1-800-FDA-1088 or www.fda.gov/medwatch.

### Drug Interactions

Moderate CYP2C9 or strong CYP3A4 inhibitors: Consider alternative agents or monitor closely for adverse reactions. (7.1)
Strong CYP3A4 inducers: Avoid concomitant use with BALVERSA. (7.1)
Moderate CYP3A4 inducers: Administer BALVERSA at a dose of 9 mg. (7.1)
Serum phosphate level-altering agents: Avoid concomitant use with agents that can alter serum phosphate levels before the initial dose modification period. (2.3, 7.1)
P-gp substrates: Separate BALVERSA administration by at least 6 hours before or after administration of P-gp substrates with narrow therapeutic indices. (7.2)

### Use in Specific Populations

Lactation: Advise not to breastfeed. (8.2)

See 17 for PATIENT COUNSELING INFORMATION and FDA-approved patient labeling.
Revised: 1/2024

### Full Prescribing Information: Contents*

1 INDICATIONS AND USAGE
2 DOSAGE AND ADMINISTRATION
   2.1 Patient Selection
   2.2 Recommended Dosage and Schedule
   2.3 Dose Modifications for Adverse Reactions
3 DOSAGE FORMS AND STRENGTHS
4 CONTRAINDICATIONS
5 WARNINGS AND PRECAUTIONS
   5.1 Ocular Disorders
   5.2 Hyperphosphatemia and Soft Tissue Mineralization
   5.3 Embryo-Fetal Toxicity
6 ADVERSE REACTIONS
   6.1 Clinical Trials Experience
7 DRUG INTERACTIONS
   7.1 Effect of Other Drugs on BALVERSA
   7.2 Effect of BALVERSA on Other Drugs
8 USE IN SPECIFIC POPULATIONS
   8.1 Pregnancy
   8.2 Lactation
   8.3 Females and Males of Reproductive Potential
   8.4 Pediatric Use
   8.5 Geriatric Use
   8.6 CYP2C9 Poor Metabolizers
11 DESCRIPTION
12 CLINICAL PHARMACOLOGY
   12.1 Mechanism of Action
   12.2 Pharmacodynamics
   12.3 Pharmacokinetics
   12.5 Pharmacogenomics
13 NONCLINICAL TOXICOLOGY
   13.1 Carcinogenesis, Mutagenesis, and Impairment of Fertility
14 CLINICAL STUDIES
   14.1 Urothelial Carcinoma with Susceptible FGFR3 Genetic Alterations
16 HOW SUPPLIED/STORAGE AND HANDLING
17 PATIENT COUNSELING INFORMATION
*Sections or subsections omitted from the full prescribing information are not listed.

### Full Prescribing Information

### 1 Indications and Usage

BALVERSA is indicated for the treatment of adult patients with locally advanced or metastatic urothelial carcinoma (mUC) with susceptible FGFR3 genetic alterations whose disease has progressed on or after at least one line of prior systemic therapy.

Select patients for therapy based on an FDA-approved companion diagnostic for BALVERSA [see Dosage and Administration (2.1) and Clinical Studies (14.1)].

### Limitations of Use

BALVERSA is not recommended for the treatment of patients who are eligible for and have not received prior PD-1 or PD-L1 inhibitor therapy [see Clinical Studies (14.1)].

### 2 Dosage and Administration

### 2.1 Patient Selection

Select patients for the treatment of locally advanced or metastatic urothelial carcinoma with BALVERSA based on the presence of susceptible FGFR3 genetic alterations in tumor specimens as detected by an FDA-approved companion diagnostic [see Clinical Studies (14.1)].

Information on FDA-approved tests for the detection of FGFR3 genetic alterations in urothelial cancer is available at: http://www.fda.gov/CompanionDiagnostics.

### 2.2 Recommended Dosage and Schedule

The recommended starting dose of BALVERSA is 8 mg (two 4 mg tablets) orally once daily, with a dose increase to 9 mg (three 3 mg tablets) once daily based on tolerability, including hyperphosphatemia, at 14 to 21 days [see Dosage and Administration (2.3)].

Swallow tablets whole with or without food. If vomiting occurs any time after taking BALVERSA, the next dose should be taken the next day. Treatment should continue until disease progression or unacceptable toxicity occurs.

If a dose of BALVERSA is missed, it can be taken as soon as possible on the same day. Resume the regular daily dose schedule for BALVERSA the next day. Extra tablets should not be taken to make up for the missed dose.

### Dose Increase Based on Serum Phosphate Levels

Assess serum phosphate levels 14 to 21 days after initiating treatment. Increase the dose of BALVERSA to 9 mg once daily if serum phosphate level is <9.0 mg/dL and there are no ocular disorders or Grade 2 or greater adverse reactions. Monitor phosphate levels monthly for hyperphosphatemia [see Pharmacodynamics (12.2)].

### 2.3 Dose Modifications for Adverse Reactions

The recommended dose modifications for adverse reactions are listed in Table 1.

TABLE 1
BALVERSA Dose Reduction Schedule

| | 1^{st} dose | 2^{nd} dose | 3^{rd} dose | 4^{th} dose | 5^{th} dose |
|---|---|---|---|---|---|
| Dose | reduction | reduction | reduction | reduction | reduction |
| 9 mg → | 8 mg | 6 mg | 5 mg | 4 mg | Stop |
| (three 3 | (two 4 mg | (two 3 mg | (one 5 mg | (one 4 mg | |
| mg tablets) | tablets) | tablets) | tablet) | tablet) | |
| 8 mg → | 6 mg | 5 mg | 4 mg | Stop | |
| (two 4 mg | (two 3 mg | (one 5 mg | (one 4 mg | | |
| tablets) | tablets) | tablet) | tablet) | | |

Table 2 summarizes recommendations for dose interruption, reduction, or discontinuation of BALVERSA in the management of specific adverse reactions.

TABLE 2
Dose Modifications for Adverse Reactions

| | |
|---|---|
| Adverse Reaction | BALVERSA Dose Modification |

Hyperphosphatemia
In all patients, restrict phosphate intake to 600-800 mg daily

| | | |
|---|---|---|
| <6.99 | mg/dL | Continue BALVERSA at current dose. |
| 7-8.99 | mg/dL | Continue BALVERSA at current dose. |
| | | Start phosphate binder with food until phosphate level |
| | | is <7 mg/dL. |
| | | Reduce the dose if serum phosphate remains ≥7 mg/dL |
| | | for a period of 2 months or if clinically necessary. |
| 9-10 | mg/dL | Withhold BALVERSA with weekly reassessments until |
| | | level returns to <7 mg/dL. Then restart BALVERSA at |
| | | the same dose level. |
| | | Start phosphate binder with food until serum phosphate |
| | | level returns to <7 mg/dL. |
| | | Reduce the dose for serum phosphate ≥9 mg/dL for a |
| | | period of 1 month or if clinically necessary. |
| >10 | mg/dL | Withhold BALVERSA with weekly reassessments until |

| | |
|---|---|
| | level returns to <7 mg/dL. Then may restart |
| | BALVERSA at the first reduced dose level. |
| | If hyperphosphatemia (≥10 mg/dL) for >2 weeks, |
| | discontinue BALVERSA permanently. |
| | Medical management of symptoms as clinically relevant. |
| Serum phosphate with | Discontinue BALVERSA permanently. |
| life-threatening | |
| consequences; urgent | |
| intervention indicated | |
| (e.g., dialysis) | |

Central Serous Retinopathy (CSR)

| | |
|---|---|
| Any | Withhold BALVERSA and perform an ophthalmic evaluation |
| | within 2 weeks: |
| | If improving within 14 days, restart BALVERSA at the |
| | current dose. |
| | If not improving within 14 days, withhold BALVERSA |
| | until improving; once improving, may resume at the |
| | next lower dose level. |
| | Upon restarting BALVERSA, monitor for recurrence every 1 to |
| | 2 weeks for a month. |
| | If recurs or has not improved after 4 weeks of withholding |
| | BALVERSA, consider permanent discontinuation. |

Other Adverse Reactions^{a}

| | |
|---|---|
| Grade 3 | Withhold BALVERSA until resolves to Grade 1 or baseline, then |
| | may resume dose level lower. |
| Grade 4 | Permanently discontinue. |

^{a}Dose adjustment graded using the National Cancer Institute Common Terminology Criteria for Adverse Events (NCI CTCAEv5.0).

### 3 Dosage Forms and Strengths

### Tablets:

3 mg: Yellow, round biconvex, film-coated, debossed with “3” on one side; and “EF” on the other side.
4 mg: Orange, round biconvex, film-coated, debossed with “4” on one side; and “EF” on the other side.
5 mg: Brown, round biconvex, film-coated, debossed with “5” on one side; and “EF” on the other side.

### 4 Contraindications

None.

### Warnings and Precautions

### 5.1 Ocular Disorders

BALVERSA can cause ocular disorders, including central serous retinopathy/retinal pigment epithelial detachment (CSR/RPED) resulting in visual field defect.

In the pooled safety population [see Adverse Reactions (6)], CSR/RPED occurred in 22% of patients treated with BALVERSA, with a median time to first onset of 46 days. In 104 patients with CSR, 40% required dose interruptions and 56% required dose reductions; 2.9% of BALVERSA-treated patients required permanent discontinuation for CSR. Of the 24 patients who restarted BALVERSA after dose interruption with or without dose reduction, 67% had recurrence and/or worsening of CSR after restarting. CSR was ongoing in 41% of the 104 patients at the time of last evaluation.

Dry eye symptoms occurred in 26% of BALVERSA-treated patients. All patients should receive dry eye prophylaxis with ocular demulcents as needed.

Perform monthly ophthalmological examinations during the first 4 months of treatment and every 3 months afterwards, and urgently at any time for visual symptoms.

Ophthalmological examination should include assessment of visual acuity, slit lamp examination, fundoscopy, and optical coherence tomography.

Withhold or permanently discontinue BALVERSA based on severity and/or ophthalmology exam findings [see Dosage and Administration (2.3)].

### 5.2 Hyperphosphatemia and Soft Tissue Mineralization

BALVERSA can cause hyperphosphatemia leading to soft tissue mineralization, cutaneous calcinosis, non-uremic calciphylaxis and vascular calcification. Increases in phosphate levels are a pharmacodynamic effect of BALVERSA [see Pharmacodynamics (12.2)].

In the pooled safety population [see Adverse Reactions (6)], increased phosphate occurred in 73% of BALVERSA-treated patients. The median onset time of increased phosphate was 16 days (range: 8-421) after initiating BALVERSA. Twenty-four percent of patients received phosphate binders during treatment with BALVERSA. Vascular calcification was observed in 0.2% of patients treated with BALVERSA.

Monitor for hyperphosphatemia throughout treatment. Restrict dietary phosphate intake (600-800 mg daily) and avoid concomitant use of agents that may increase serum phosphate levels.

If serum phosphate is above 7.0 mg/dL, consider adding an oral phosphate binder until serum phosphate level returns to <7.0 mg/dL. Withhold, dose reduce, or permanently discontinue BALVERSA based on duration and severity of hyperphosphatemia according to Table 2 [see Dosage and Administration (2.3)].

### 5.3 Embryo-Fetal Toxicity

Based on the mechanism of action and findings in animal reproduction studies, BALVERSA can cause fetal harm when administered to a pregnant woman. In an embryo-fetal toxicity study, oral administration of erdafitinib to pregnant rats during the period of organogenesis caused malformations and embryo-fetal death at maternal exposures that were less than the human exposures at the maximum human recommended dose based on area under the curve (AUC). Advise pregnant women of the potential risk to the fetus. Advise female patients of reproductive potential to use effective contraception during treatment with BALVERSA and for one month after the last dose. Advise male patients with female partners of reproductive potential to use effective contraception during treatment with BALVERSA and for one month after the last dose [see Use in Specific Populations (8.1, 8.3) and Clinical Pharmacology (12.1)].

### 6 Adverse Reactions

The following serious adverse reactions are also described elsewhere in the labeling:
Ocular Disorders [see Warnings and Precautions (5.1)].
Hyperphosphatemia [see Warnings and Precautions (5.2)].

### 6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

The pooled safety population described in the WARNINGS AND PRECAUTIONS reflects exposure to BALVERSA as a single agent at the recommended dose (8 to 9 mg orally daily) in 479 patients with advanced urothelial cancer and FGFR alterations in 42756493BLC3001 (NCT03390504), 42756493BLC2001 (NCT02365597), 42756493BLC2002 (NCT 03473743), and 42756493EDI1001 (NCT01703481). Among 479 patients who received BALVERSA, the median duration of treatment was 4.8 months (range: 0.1 to 43 months). In this pooled safety population, the most common (>20%) adverse reactions, including laboratory abnormalities, were increased phosphate, nail disorders, stomatitis, diarrhea, increased creatinine, increased alkaline phosphatase, increased alanine aminotransferase, decreased hemoglobin, decreased sodium, increased aspartate aminotransferase, fatigue, dry mouth, dry skin, decreased phosphate, decreased appetite, dysgeusia, constipation, increased calcium, dry eye, palmar-plantar erythrodysesthesia syndrome, increased potassium, alopecia, and central serous retinopathy.

### BLC3001

The safety of BALVERSA was evaluated in Cohort 1 of the BLC3001 study that included patients with locally advanced unresectable or metastatic urothelial carcinoma which had susceptible FGFR3 genetic alterations and were previously treated with a PD-1 or PD-L1 inhibitor [see Clinical Studies (14.1)]. Patients received either BALVERSA (8 mg orally once daily with individualized up-titration to 9 mg) (n=135) or chemotherapy (docetaxel 75 mg/m² once every 3 weeks or vinflunine 320 mg/m² once every 3 weeks) (n=112). Among patients who received BALVERSA, median duration of treatment was 4.8 months (range: 0.2 to 38 months).

Serious adverse reactions occurred in 41% of patients who received BALVERSA. Serious reactions in >2% of patients included urinary tract infection (4.4%), hematuria (3.7%), hyponatremia (2.2%), and acute kidney injury (2.2%). Fatal adverse reactions occurred in 4.4% of patients who received BALVERSA, including sudden death (1.5%), pneumonia (1.5%), renal failure (0.7%), and cardiorespiratory arrest (0.7%).

Permanent discontinuation of BALVERSA due to an adverse reaction occurred in 14% of patients. Adverse reactions which resulted in permanent discontinuation of BALVERSA in >2% of patients included nail disorders (3%) and eye disorders (2.2%).

Dosage interruptions of BALVERSA due to an adverse reaction occurred in 72% of patients. Adverse reactions which required dosage interruption in >4% of patients included nail disorders (22%), stomatitis (19%), eye disorders (16%), palmar-plantar erythrodysesthesia syndrome (15%), diarrhea (10%), hyperphosphatemia (7%), increased aspartate aminotransferase (6%), and increased alanine aminotransferase (5%).

Dose reductions of BALVERSA due to an adverse reaction occurred in 69% of patients. Adverse reactions which required dose reductions in >4% of patients included nail disorders (27%), stomatitis (19%), eye disorders (17%), palmar-plantar erythrodysesthesia syndrome (12%), diarrhea (7%), dry mouth (4.4%), and hyperphosphatemia (4.4%).

Table 3 presents adverse reactions reported in ≥15% of patients treated with BALVERSA at 8 or 9 mg once daily versus chemotherapy.

TABLE 3
Adverse Reactions Reported in ≥15% of Patients Who Received
BALVERSA Versus Chemotherapy (Study BLC3001)

| | BALVERSA | | Chemotherapy | |
|---|---|---|---|---|
| | (N = 135) | | (N = 112) | |
| | All | Grade | All | Grade |
| | Grades | 3-4 | Grades | 3-4 |
| Adverse Reaction | (%) | (%) | (%) | (%) |
| Skin and subcutaneous | | | | |
| tissue disorders | | | | |
| Nail disorders^{a} | 70 | 12 | 5 | 0 |
| Palmar-plantar | 30 | 10 | 0.9 | 0 |
| erythrodysesthesia | | | | |
| syndrome | | | | |
| Dry Skin^{a} | 27 | 1.5 | 6 | 0 |
| Alopecia | 25 | 0.7 | 24 | 0 |
| Gastrointestinal disorders | | | | |
| Diarrhea^{a} | 63 | 3 | 17 | 2.7 |
| Stomatitis^{a} | 56 | 10 | 18 | 1.8 |
| Dry Mouth | 39 | 0 | 3.6 | 0 |
| Constipation | 27 | 0 | 28 | 1.8 |
| Nervous system disorders | | | | |
| Dysgeusiaa | 30 | 0.7 | 7 | 0 |
| General disorders | | | | |
| Fatigue^{a} | 29 | 1.5 | 42 | 7 |
| Metabolism and nutrition | | | | |
| disorders | | | | |
| Decreased appetite | 27 | 3 | 21 | 2.7 |
| Eye disorders | | | | |
| Dry eye^{a} | 25 | 0.7 | 3.6 | 0 |
| Central serous retinopathy^{a} | 18 | 2.2 | 0 | 0 |
| Investigations | | | | |
| Decreased weight | 22 | 2 | 2.7 | 0 |

^{a}Includes multiple terms

Clinically relevant adverse reactions in <15% of patients who received BALVERSA included nausea (15%), pyrexia (15%), epistaxis (13%), vomiting (10%), and arthralgia (10%).

Table 4 presents laboratory abnormalities reported in ≥15% of patients treated with BALVERSA at 8 or 9 mg once daily versus chemotherapy.

TABLE 4
Selected Laboratory Abnormalities Reported in ≥15% of
Patients Who Received BALVERSA Versus Chemotherapy;
Cohort 1 Safety Analysis Set (Study BLC3001)

| | BALVERSA | | Chemotherapy | |
|---|---|---|---|---|
| | (N = 135¹) | | (N = 112²) | |
| | All | Grade | All | Grade |
| | Grades³ | 3-4³ | Grades³ | 3-4³ |
| Laboratory Abnormality | (%) | (%) | (%) | (%) |
| Chemistry | | | | |
| Increased phosphate | 76 | 5 | 0 | 0 |
| Increased alkaline | 54 | 4.7 | 29 | 1 |
| phosphatase | | | | |
| Increased alanine | 46 | 3.8 | 15 | 1 |
| aminotransferase | | | | |
| Increased aspartate | 44 | 3.1 | 13 | 0 |
| aminotransferase | | | | |
| Decreased sodium | 44 | 16 | 25 | 6 |
| Increased creatinine | 43 | 1.5 | 17 | 0 |
| Decreased phosphate | 34 | 8 | 25 | 3.6 |
| Increased calcium | 27 | 8 | 9 | 0 |
| Increased potassium | 24 | 0 | 21 | 0 |
| Hematology | | | | |
| Decreased hemoglobin | 50 | 12 | 57 | 12 |
| Decreased platelet count | 17 | 1.5 | 18 | 1 |
| Decreased neutrophil | 16 | 0.8 | 40 | 26 |
| count | | | | |

¹The denominator used to calculate the rate varied from 52 to 131 based on the number of patients with a baseline value and at least one post-treatment value.
²The denominator used to calculate the rate varied from 11 to 102 based on the number of patients with a baseline value and at least one post-treatment value.
³Severity graded per NCI CTCAE v4.03.

### BLC2001

The safety of BALVERSA was evaluated in the BLC2001 study that included 87 patients with locally advanced or metastatic urothelial carcinoma which had susceptible FGFR3 and other FGFR alterations, and which progressed during or following at least one line of prior chemotherapy including within 12 months of neoadjuvant or adjuvant chemotherapy [see Clinical Studies (14.1)]. Patients were treated with BALVERSA at 8 mg orally once daily; with a dose increase to 9 mg in patients with phosphate levels <5.5 mg/dL on Day 14 of Cycle 1. Median duration of treatment was 5.3 months (range: 0 to 17 months). Serious adverse reactions occurred in 41% of patients. The most frequent (≥3%) serious adverse reactions were central serous retinopathy (4.6%), urinary tract infection (3.4%), and general physical health deterioration (3.4%).

Fatal adverse reactions occurred in 8% of patients, including acute myocardial infarction (1.1%).

Permanent discontinuation of BALVERSA due to an adverse reaction occurred in 21% of patients. The most frequent (≥2%) reasons for permanent discontinuation included central serous retinopathy (4.6%), general physical health deterioration (3.4%), palmar-plantar erythrodysesthesia syndrome (2.3%), acute kidney injury (2.3%), and fatigue (2.3%).

Dosage interruptions of BALVERSA occurred in 68% of patients. The most frequent (>5%) adverse reactions requiring dosage interruption included hyperphosphatemia (24%), stomatitis (17%), nail disorders (16%), central serous retinopathy (9%), palmar-plantar erythro-dysesthesia syndrome (8%), and fatigue (8%).

Dose reductions of BALVERSA occurred in 53% of patients. The most frequent (≥5%) adverse reactions for dose reductions included nail disorders (21%), stomatitis (15%), central serous retinopathy (14%), hyperphosphatemia (7%), palmar-plantar erythro-dysesthesia syndrome (7%), fatigue (6%), and blurred vision (6%).

Table 5 presents adverse reactions reported in ≥15% of patients treated with BALVERSA at 8 mg or 9 mg once daily.

TABLE 5
Adverse Reactions Reported in ≥15% of Patients
(Study BLC2001)

| | BALVERSA 8 mg daily | |
|---|---|---|
| | (N = 87) | |
| | All | Grade |
| | Grades | 3-4 |
| Adverse Reaction | (%) | (%) |
| Gastrointestinal disorders | | |
| Stomatitis^{a} | 62 | 11 |
| Diarrhea^{a} | 48 | 4.6 |
| Dry mouth | 45 | 0 |
| Constipation | 28 | 1.1 |
| Nausea | 21 | 1. |
| Skin and subcutaneous tissue | | |
| disorders | | |
| Nail disorders^{a} | 62 | 14 |
| Dry skin^{a} | 37 | 0 |
| Alopecia | 26 | 0 |
| Palmar-plantar | 26 | 6 |
| erythrodysesthesia syndrome | | |
| General disorders and admin. | | |
| site conditions | | |
| Fatigue^{a,b} | 54 | 8 |
| Decreased weight | 16 | 0 |
| Metabolism and nutrition | | |
| disorders | | |
| Decreased appetite | 38 | 0.0 |
| Nervous system disorders | | |
| Dysgeusia^{a} | 38 | 1.1 |
| Eye disorders | | |
| Dry eyeª | 29 | 1.1 |
| Central serous retinopathy^{a} | 28 | 4.6 |
| Blurred vision | 17 | 0 |
| Infections and Infestations | | |
| Urinary tract infection | 17 | 6 |

^{a}Includes multiple terms
^{b}Includes fatal adverse reactions (n = 2)

Clinically relevant adverse reactions in <15% of patients who received BALVERSA included pyrexia (14%), extremity pain (13%), vomiting (13%), and peripheral edema (10%).

Table 6 presents laboratory abnormalities reported in >15% of patients treated with BALVERSA at 8 mg or 9 mg once daily.

TABLE 6
Selected Laboratory Abnormalities Reported
in ≥15% of Patients

| | BALVERSA 8 mg daily | |
|---|---|---|
| | (N = 87¹) | |
| | All | Grade |
| | Grades | 3-4 |
| Laboratory Abnormality | (%) | (%) |
| Chemistry | | |
| Increased phosphate | 76 | 1.2 |
| Increased creatinine | 52 | 4.7 |
| Increased alanine | 41 | 1.2 |
| aminotransferase | | |
| Increased alkaline | 41 | 1.2 |
| phosphatase | | |
| Decreased sodium | 40 | 16 |
| Decreased magnesium | 31 | 1.2 |
| Increased aspartate | 30 | 0 |
| aminotransferase | | |
| Decreased phosphate | 24 | 9 |
| Increased calcium | 22 | 3.5 |
| Hematology | | |
| Decreased hemoglobin | 35 | 3.5 |
| Decreased platelets | 19 | 1.2 |
| Decreased leukocytes | 17 | 0 |

¹The denominator used to calculate the rate varied from 83 to 86 based on the number of patients with a baseline value and at least one post-treatment value.

### 7 Drug Interactions

### 7.1 Effect of Other Drugs on BALVERSA

Table 7 summarizes drug interactions that affect the exposure of BALVERSA or serum phosphate level and their clinical management.

TABLE 7
Drug Interactions that Affect BALVERSA
Moderate CYP2C9 or Strong CYP3A4 Inhibitors

| | |
|---|---|
| Clinical | Co-administration of BAL VERSA with moderate |
| Impact | CYP2C9 or strong CYP3A4 inhibitors increased |
| | erdafitinib plasma concentrations [see Clinical |
| | Pharmacology (12.3)]. |
| | Increased erdafitinib plasma concentrations may lead to |
| | increased drug-related toxicity [see Warnings and |
| | Precautions (5)]. |
| Clinical | Consider alternative therapies that are not moderate |
| Management | CYP2C9 or strong CYP3A4 inhibitors during treatment |
| | with BALVERSA. |
| | If co-administration of a moderate CYP2C9 or strong |
| | CYP3A4 inhibitor is unavoidable, monitor closely for |
| | adverse reactions and consider dose modifications |
| | accordingly [see Dosage and Administration (2.3)]. If |
| | the moderate CYP2C9 or strong CYP3A4 inhibitor is |
| | discontinued, resume the BAL VERSA dose before dose |
| | modifications in the absence of drug-related toxicity. |

Strong CYP3A4 Inducers

| | |
|---|---|
| Clinical | Co-administration of BALVERSA with strong CYP3A4 |
| Impact | inducers decreased erdafitinib plasma concentrations |
| | [see Clinical Pharmacology (12.3)]. |
| | Decreased erdafitinib plasma concentrations may lead to |
| | decreased activity. |
| Clinical | Avoid co-administration of strong CYP3A4 inducers |
| Management | with BALVERSA. |

Moderate CYP3A4 Inducers

| | |
|---|---|
| Clinical | Co-administration of BALVERSA with moderate |
| Impact | CYP3A4 inducers may decrease erdafitinib plasma |
| | concentrations/see Clinical Pharmacology (12.3)]. |
| | Decreased erdafitinib plasma concentrations may lead to |
| | decreased activity. |
| Clinical | If a moderate CYP3A4 inducer must be co-administered |
| Management | at the start of BALVERSA treatment, administer |
| | BALVERSA at a dose of 9 mg daily. |
| | When a moderate CYP3A4 inducer is discontinued, |
| | continue BALVERSA at the same dose, in the absence of |
| | drug-related toxicity. |

Serum Phosphate Level-Altering Agents

| | |
|---|---|
| Clinical | Co-administration of BALVERSA with other serum |
| Impact | phosphate level-altering agents may increase or decrease |
| | serum phosphate levels [see Pharmacodynamics (12.2)]. |
| | Changes in serum phosphate levels due to serum |
| | phosphate level-altering agents (other than erdafitinib) |
| | may interfere with serum phosphate levels needed for the |
| | determination of initial dose increased based on serum |
| | phosphate levels [see Dosage and Administration (2.3)]. |
| Clinical | Avoid co-administration of serum phosphate level- |
| Management | altering agents with BALVERSA before initial dose |
| | increase period based on serum phosphate levels (Days |
| | 14 to 21) [see Dosage and Administration (2.3)]. |

### 7.2 Effect of BALVERSA on Other Drugs

Table 8 summarizes the effect of BALVERSA on other drugs and their clinical management.

TABLE 8
BALVERSA Drug Interactions that Affect Other Drugs
P-glycoprotein (P-gp) Substrates

| | |
|---|---|
| Clinical | Co-administration of BALVERSA with P-gp |
| Impact | substrates may increase the plasma concentrations of |
| | P-gp substrates [see Clinical Pharmacology (12.3)]. |
| | Increased plasma concentrations of P-gp substrates |
| | may lead to increased toxicity of the P-gp substrates. |
| Clinical | If co-administration of BALVERSA with P-gp |
| Management | substrates is unavoidable, separate BALVERSA |
| | administration by at least 6 hours before or after |
| | administration of P-gp substrates with narrow |
| | therapeutic index. |

### 8 Use in Specific Populations

### 8.1 Pregnancy

### Risk Summary

Based on the mechanism of action and findings in animal reproduction studies, BALVERSA can cause fetal harm when administered to a pregnant woman [see Clinical Pharmacology (12.1)]. There are no available data on BALVERSA use in pregnant women to inform a drug-associated risk. Oral administration of erdafitinib to pregnant rats during organogenesis caused malformations and embryo-fetal death at maternal exposures that were less than the human exposures at the maximum recommended human dose based on AUC (see Data). Advise pregnant women and females of reproductive potential of the potential risk to the fetus.

The estimated background risk of major birth defects and miscarriage for the indicated population is unknown. All pregnancies have a background risk of birth defect, loss, or other adverse outcomes. In the U.S. general population, the estimated background risk of major birth defects and miscarriage in clinically recognized pregnancies is 2-4% and 15-20%, respectively.

### Data

### Animal Data

In an embryo-fetal toxicity study, erdafitinib was orally administered to pregnant rats during the period of organogenesis. Doses ≥4 mg/kg/day (at total maternal exposures <0.1% of total human exposures at the maximum recommended human dose based on AUC) produced embryo-fetal death, major blood vessel malformations and other vascular anomalies, limb malformations (ectrodactyly, absent or misshapen long bones), an increased incidence of skeletal anomalies in multiple bones (vertebrae, sternebrae, ribs), and decreased fetal weight.

### 8.2 Lactation

### Risk Summary

There are no data on the presence of erdafitinib in human milk, or the effects of erdafitinib on the breastfed child, or on milk production. Because of the potential for serious adverse reactions from erdafitinib in a breastfed child, advise lactating women not to breastfeed during treatment with BALVERSA and for one month following the last dose.

### 8.3 Females and Males of Reproductive Potential

BALVERSA can cause fetal harm when administered to a pregnant woman [see Use in Specific Populations (8.1)].

### Pregnancy Testing

Verify pregnancy status in females of reproductive potential prior to initiating treatment with BALVERSA.

### Contraception

### Females

Advise females of reproductive potential to use effective contraception during treatment with BALVERSA and for one month after the last dose.

### Males

Advise male patients with female partners of reproductive potential to use effective contraception during treatment with BALVERSA and for one month after the last dose.

### Infertility

### Females

Based on findings from animal studies, BALVERSA may impair fertility in females of reproductive potential [see Nonclinical Toxicology (13.1)].

### 8.4 Pediatric Use

Safety and effectiveness of BALVERSA in pediatric patients have not been established.

In 4 and 13-week repeat-dose toxicology studies in rats and dogs, toxicities in bone and teeth were observed at an exposure less than the human exposure (AUC) at the maximum recommended human dose. Chondroid dysplasia/metaplasia were reported in multiple bones in both species, and tooth abnormalities included abnormal/irregular denting in rats and dogs and discoloration and degeneration of odontoblasts in rats.

### 8.5 Geriatric Use

Of the 479 patients treated with BALVERSA in clinical studies, 40% of patients were less than 65 years old, 40% of patients were 65 years to 74 years old, and 20% were 75 years old and over.

Patients 65 years of age and older treated with BALVERSA experienced a higher incidence of adverse reactions requiring treatment discontinuation than younger patients. In clinical trials, the incidence of treatment discontinuations of BALVERSA due to adverse reactions was 10% in patients younger than 65 years, 20% in patients ages 65-74 years, and 35% in patients 75 years or older.

No overall difference in efficacy was observed between these patients and younger patients [see Clinical Studies (14.1)].

### 8.6 CYP2C9 Poor Metabolizers

CYP2C9*3/*3 Genotype: Erdafitinib plasma concentrations are predicted to be higher in patients with the CYP2C9*3/*3 genotype. Monitor for increased adverse reactions in patients who are known or suspected to have CYP2C9*3/*3 genotype [see Pharmacogenomics (12.5)].

### 11 Description

Erdafitinib, the active ingredient in BALVERSA, is a kinase inhibitor. The chemical name is N-(3,5-dimethoxyphenyl)-N′-(1-methylethyl)-N-[3-(1-methyl-1H-pyrazol-4-yl)quinoxalin-6-yl]ethane-1,2-diamine. Erdafitinib is a yellow powder. It is practically insoluble, or insoluble to freely soluble in organic solvents, and slightly soluble to practically insoluble, or insoluble in aqueous media over a wide range of pH values. The molecular formula is C₂₅H₃₀N₆O₂ and molecular weight is 446.56.

Chemical structure of erdafitinib is as follows:

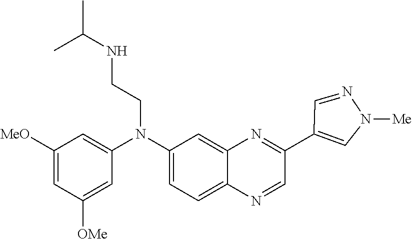 [SMILES: COc1cc(OC)cc(N(CCNC(C)C)c2ccc3ncc(-c4cnn(C)c4)nc3c2)c1]

BALVERSA® (erdafitinib) tablets are supplied as 3 mg, 4 mg or 5 mg film-coated tablets for oral administration and contains the following inactive ingredients:

Tablet Core: Croscarmellose sodium, Magnesium stearate (from vegetable source), Mannitol, Meglumine, and Microcrystalline Cellulose.

Film Coating: (Opadry amb II): Glycerol monocaprylocaprate Type I, Polyvinyl alcohol-partially hydrolyzed, Sodium lauryl sulfate, Talc, Titanium dioxide, Iron oxide yellow, Iron oxide red (for the orange and brown tablets only), Ferrosoferric oxide/iron oxide black (for the brown tablets only).

### 12 Clinical Pharmacology

### 12.1 Mechanism of Action

Erdafitinib is a kinase inhibitor that binds to and inhibits enzymatic activity of FGFR1, FGFR2, FGFR3 and FGFR4 based on in vitro data. Erdafitinib inhibited FGFR phosphorylation and signaling and decreased cell viability in cell lines expressing FGFR genetic alterations, including point mutations, amplifications, and fusions. Erdafitinib demonstrated antitumor activity in FGFR-expressing cell lines and xenograft models derived from tumor types, including bladder cancer.

### 12.2 Pharmacodynamics

### Cardiac Electrophysiology

Based on evaluation of QTc interval in an open-label, dose escalation and dose expansion study in 187 patients with cancer, erdafitinib had no large effect (i.e., ≥20 ms) on the QTc interval.

### Serum Phosphate

FGFR inhibition by BALVERSA increases serum phosphate level [see Dosage and Administration (2.3) and Drug Interactions (7.1)].

### 12.3 Pharmacokinetics

Following administration of BALVERSA 8 mg once daily, the mean (coefficient of variation [CV %]) erdafitinib steady-state maximum plasma concentration (Cₘₐₓ), area under the curve (AUCₜₐᵤ), and minimum plasma concentration (Cₘᵢₙ) were 1,399 ng/mL (51%), 29,268 ng·h/mL (60%), and 936 ng/mL (65%), respectively.

Following single and repeat once daily dosing of BALVERSA, erdafitinib exposure (C max and AUC) increased proportionally across the dose range of 0.5 to 12 mg (0.06 to 1.3 times the maximum approved recommended dose). Steady state was achieved after 2 weeks with once daily dosing with a mean accumulation ratio was 4-fold.

### Absorption

Median time to achieve peak plasma concentration (t max) was 2.5 hours (range: 2 to 6 hours).

### Effect of Food

No clinically meaningful differences in erdafitinib exposure was observed following administration of BALVERSA with a high-fat and high-calorie meal (800 calories to 1,000 calories with approximately 50% of total caloric content of the meal from fat).

### Distribution

The mean apparent volume of distribution of erdafitinib was 29 L.

Erdafitinib protein binding was 99.7% in patients, primarily to alpha-1-acid glycoprotein.

### Elimination

The mean total apparent clearance (CL/F) of erdafitinib was 0.362 L/h.

The mean effective half-life of erdafitinib was 59 hours.

### Metabolism

Erdafitinib is primarily metabolized by CYP2C9 and CYP3A4. The contribution of CYP2C9 and CYP3A4 in the total clearance of erdafitinib is estimated to be 39% and 20%, respectively. Unchanged erdafitinib was the major drug-related moiety in plasma, there were no circulating metabolites.

### Excretion

Following a single oral dose of radiolabeled erdafitinib, approximately 69% of the dose was recovered in feces (19% as unchanged) and 19% in urine (13% as unchanged).

### Specific Populations

No clinically meaningful effects on erdafitinib exposure were observed based on age (21-92 years), sex, race (White, Hispanic or Asian), body weight (36-166 kg), mild (Child-Pugh A) or moderate (Child-Pugh B) hepatic impairment, or mild to moderate renal impairment (eGFR 30 to 89 mL/min/1.73 m²). Limited data are available in patients with severe (Child-Pugh C) hepatic impairment and in patients with severe renal impairment. The pharmacokinetics of erdafitinib in patients with renal impairment requiring dialysis is unknown.

### Drug Interaction Studies

### Clinical Studies

### Effect of Other Drugs on Erdafitinib

### Moderate CYP2C9 Inhibitors

Erdafitinib mean ratios for Cₘₐₓ and AUC_{inf} were 121% and 148%, respectively, when BALVERSA was co-administered with fluconazole, a moderate CYP2C9 and CYP3A4 inhibitor, relative to BALVERSA administered alone.

### Strong CYP3A4 Inhibitors

Erdafitinib mean ratios for Cₘₐₓ and AUC_{inf} were 105% and 134%, respectively, when BALVERSA was co-administered with itraconazole (a strong CYP3A4 inhibitor and P-gp inhibitor) relative to BALVERSA alone.

### CYP3A4 2C9 Inducers

Erdafitinib mean ratios for Cₘₐₓ and AUC_{inf} were 78% and 45%, respectively, when BALVERSA was co-administered with carbamazepine (a strong CYP3A4 and weak CYP2C9 inducer) relative to BALVERSA alone.

### Effect of Erdafitinib on Other Drugs

### CYP3A4 Substrates:

No clinically meaningful effect on the exposure of midazolam (a CYP3A4 substrate) was observed following coadministration with BALVERSA.

### OCT2 Substrates:

No clinically meaningful effect on the exposure of metformin (an OCT2 substrate) was observed following coadministration with BALVERSA.

### In Vitro Studies

### CYP Substrates

Erdafitinib is a time dependent inhibitor and inducer of CYP3A4. Erdafitinib is not an inhibitor of other major CYP isozymes at clinically relevant concentrations.

### Transporters

Erdafitinib is a substrate and inhibitor of P-gp. P-gp inhibitors are not expected to affect erdafitinib exposure to a clinically relevant extent. Erdafitinib is an inhibitor of OCT2.

Erdafitinib does not inhibit BCRP, OATP1B, OATP1B3, OAT1, OAT3, OCT1, MATE-1, or MATE-2K at clinically relevant concentrations.

### Acid-Lowering Agents

Erdafitinib has adequate solubility across the pH range of 1 to 7.4. Acid-lowering agents (including antacids, H₂-antagonists and proton pump inhibitors) are not expected to affect the bioavailability of erdafitinib.

### 12.5 Pharmacogenomics

CYP2C9 activity is reduced in individuals with genetic variants, such as the CYP2C9*2 and CYP2C9*3 polymorphisms. Erdafitinib exposure was similar in subjects with CYP2C9*1/*2 and *1/*3 genotypes relative to subjects with CYP2C9*1/*1 genotype (wild type). No data are available in subjects characterized by other genotypes (e.g., *2/*2, *2/*3, *3/*3). Simulation suggested no clinically meaningful differences in erdafitinib exposure in subjects with CYP2C9*2/*2 and *2/*3 genotypes. The exposure of erdafitinib is predicted to be 50% higher in subjects with the CYP2C9*3/*3 genotype, estimated to be present in 0.4% to 3% of the population among various ethnic groups.

### 13 Nonclinical Toxicology

### 13.1 Carcinogenesis, Mutagenesis, and Impairment of Fertility

Carcinogenicity studies have not been conducted with erdafitinib.

Erdafitinib was not mutagenic in a bacterial reverse mutation (Ames) assay and was not clastogenic in an in vitro micronucleus or an in vivo rat bone marrow micronucleus assay.

Fertility studies in animals have not been conducted with erdafitinib. In the 3-month repeat-dose toxicity study, erdafitinib showed effects on female reproductive organs (necrosis of the ovarian corpora lutea) in rats at an exposure less than the human exposure (AUC) at maximum recommended human dose.

### 14 Clinical Studies

14.1 Urothelial Carcinoma with Susceptible FGFR3 Genetic Alterations

The efficacy of BALVERSA was evaluated in Study BLC3001 (NCT03390504) Cohort 1, a randomized, open-label, multicenter study in which 266 patients with advanced urothelial cancer harboring selected FGFR3 alterations were randomized 1:1 to receive BALVERSA (8 mg with titration up to 9 mg) versus chemotherapy (docetaxel 75 mg/m² once every 3 weeks or vinflunine 320 mg/m² once every 3 weeks) until unacceptable toxicity or progression. Randomization was stratified by region (North America vs. Europe vs. rest of world), Eastern Cooperative Oncology Group (ECOG) performance status (0 or 1 vs. 2) and visceral or bone metastases (yes vs. no). All patients needed to have had disease progression after 1 or 2 prior treatments, at least 1 of which included a PD-1 or PD-L1 inhibitor. FGFR3 genetic alterations were identified from tumor tissue in a central laboratory by the QIAGEN Therascreen® FGFRRGQ RT-Polymerase Chain Reaction (PCR) kit in 75% of patients while the remainder (25%) were identified by local next generation sequencing (NGS) assays.

The major efficacy outcome measures were overall survival (OS), progression-free survival (PFS), and objective response rate (ORR) assessed by investigator using RECIST (Response Evaluation Criteria in Solid Tumors) Version 1.1.

The median age was 67 years (range: 32 to 86 years) and 71% were male; 54% were White, 29% Asian, 0.4% Black, 0.4% multiple races, 16% not reported; 2% were Hispanic/Latino; and baseline ECOG performance status was 0 (43%), 1 (48%), or 2 (9%). Eighty-one percent of patients had FGFR3 mutations, 17% had fusions, and 2% had both mutations and fusions. Ninety-five percent of patients had pure transitional cell carcinoma (TCC) and 5% had TCC with other histologic variants. The primary tumor location was the upper tract for 33% of subjects and lower tract for 67%; 74% of patients had visceral or bone metastases. Eighty-eight percent of patients received platinum-containing chemotherapy previously. PD-1 or PD-L1 inhibitor therapy was received only in the neoadjuvant or adjuvant setting in 7% of patients.

Statistically significant improvements in OS, PFS, and ORR were demonstrated for BALVERSA compared with chemotherapy.

Table 9 and FIGS. 1 and 2 summarize the efficacy results for BLC3001 Cohort 1.

TABLE 9
Efficacy Results for Study BLC3001 Cohort 1

| | BALVERSA | Chemotherapy |
|---|---|---|
| | N = 136 | N = 130 |
| Overall Survival (OS) | | |
| Number of events (%) | 77 (56.6%) | 78 (60.0%) |
| Median^{a}, months | 12.1 (10.3, 16.4) | 7.8 (6.5, 11.1) |
| (95% Cl) | | |
| Hazard ratio^{b} (95% Cl) | 0.64 (0.47, 0.88) | |
| p-value^{c} | 0.0050 | |
| Progression-free survival | | |
| (PFS) | | |
| Number of events (%) | 101 (74.3%) | 90 (69.2%) |
| Median^{a}, months | 5.6 (4.4, 5.7) | 2.7 (1.8, 3.7) |
| (95% Cl) | | |
| Hazard ratio^{b} (95% Cl) | 0.58 (0.44, 0.78) | |
| p-value^{c} | 0.0002 | |
| Objective response rate | | |
| (ORR) | | |
| ORR (95% Cl) | 35.3% (27.3, 43.9) | 8.5% (4.3, 14.6) |
| p-value^{d} | <0.001 | |
| Complete response, CR (%) | 5.1% | 0.8% |
| Partial response, PR (%) | 30.1% | 7.7% |

All p-values reported are 2-sided and compared with 0.019 of the allocated alpha for the interim analysis.
^{a}Based on Kaplan-Meier estimates
^{b}Based on an unstratified Cox proportional hazard model
^{c}Based on an unstratified log-rank test
^{d}p-value is estimated using Cochran-Haenszel (CMH) test with ECOG performance status (0 or 1 vs 2) as a stratification factor.
ORR = confirmed objective response (CR + PR)
CI = Confidence Interval

### Study BLC3001 Cohort 2

Study BLC3001 (NCT03390504) Cohort 2 was a multicenter, open-label, randomized study in 351 patients with locally advanced or metastatic urothelial carcinoma with selected FGFR3 alterations who received 1 prior line of systemic therapy and no prior PD-1 or PD-L1 inhibitor. Patients were randomized 1:1 to receive BALVERSA (8 mg with titration up to 9 mg) or pembrolizumab 200 mg every 3 weeks. The study did not meet its major efficacy outcome measure for superiority of OS at the pre-specified final analysis. The OS hazard ratio (HR) was 1.18 (95% CI: 0.92, 1.51; p=0.18), median 10.9 (95% CI: 9.2, 12.6) months for BALVERSA versus 11.1 (95% CI: 9.7, 13.6) months for pembrolizumab [see Indications and Usage (1)].

### Study BLC2001

Study BLC2001 (NCT02365597) was a multicenter, open-label, single-arm study to evaluate the efficacy and safety of BALVERSA in patients with locally advanced or metastatic urothelial carcinoma (mUC). FGFR mutation status for screening and enrollment of patients was determined by a clinical trial assay (CTA). The efficacy population consists of a cohort of eighty-seven patients who were enrolled in this study with disease that had progressed on or after at least one prior chemotherapy and that had at least 1 of the following genetic alterations: FGFR3 gene mutations (R248C, S249C, G370C, Y373C) or FGFR gene fusions (FGFR3-TACC3, FGFR3-BAIAP2L1, FGFR2-BICC1, FGFR2-CASP7), as determined by the CTA performed at a central laboratory. Tumor samples from 69 patients were tested retrospectively by the QIAGEN Therascreen® FGFRRGQ RT-PCR Kit, which is the FDA-approved test for selection of patients with mUC for BALVERSA.

Patients received a starting dose of BALVERSA at 8 mg once daily with a dose increase to 9 mg once daily in patients whose serum phosphate levels were below the target of 5.5 mg/dL between days 14 and 17; a dose increase occurred in 41% of patients. BALVERSA was administered until disease progression or unacceptable toxicity. The major efficacy outcome measures were ORR and duration of response (DoR), as determined by blinded independent review committee (BIRC) according to RECIST v1.1.

The median age was 67 years (range: 36 to 87 years), 79% were male, and 74% were Caucasian. Most patients (92%) had a baseline Eastern Cooperative Oncology Group (ECOG) performance status of 0 or 1. Sixty-six percent of patients had visceral metastases. Eighty-four (97%) patients received at least one of cisplatin or carboplatin previously. Fifty-six percent of patients only received prior cisplatin-based regimens, 29% received only prior carboplatin-based regimens, and 10% received both cisplatin and carboplatin-based regimens. Three (3%) patients had disease progression following prior platinum-containing neoadjuvant or adjuvant therapy only. Twenty-four percent of patients had been treated with prior anti PD-L1/PD-1 therapy.

Efficacy results are summarized in Table 10 and Table 11. ORR was 32.2%. Responders included patients who had previously not responded to anti PD-L1/PD-1 therapy.

TABLE 10
Efficacy Results

| | BIRC^{a} Assessment |
|---|---|
| Endpoint | N = 87 |
| ORR (95% C1) | 32.2% (22.4, 42.0) |
| Complete response (CR) | 2.3% |
| Partial response (PR) | 29.9% |
| Median DoR in months | 5.4 (4.2, 6.9) |
| (95% Cl) | |

^{a}BIRC: Blinded Independent Review Committee
ORR = CR + PR
Cl = Confidence Interval

TABLE 11
Efficacy Results by FGFR Genetic Alteration

| | BIRC^{a} Assessment |
|---|---|
| FGFR3 Point Mutation | N = 64 |
| ORR (95% C1) | 40.6% (28.6, 52.7) |
| FGFR3 Fusion^{b,c} | N = 18 |
| ORR (95% C1) | 11.1% (0, 25.6) |
| FGFR2 Fusionc | N = 6 |
| ORR | 0 |

^{a}BIRC: Blinded Independent Review Committee
^{b}Both responders had FGFR3-TACC3 V1 fusion
^{c}One patient with a FGFR2-CASP7/FGFR3-TACC3 V3 fusion isreported in both FGFR2 fusion and FGFR3 fusion above
ORR = CR + PR
Cl = Confidence Interval

### 16 how Supplied/Storage and Handling

BALVERSA® (erdafitinib) tablets are available in the strengths and packages listed below:
3 mg tablets: Yellow, round biconvex, film-coated, debossed with “3” on one side and “EF” on the other side.
Bottle of 56-tablets with child resistant closure (NDC 59676-030-56).
Bottle of 84-tablets with child resistant closure (NDC 59676-030-84).
4 mg tablets: Orange, round biconvex, film-coated, debossed with “4” on one side and “EF” on the other side.
Bottle of 28-tablets with child resistant closure (NDC 59676-040-28).
Bottle of 56-tablets with child resistant closure (NDC 59676-040-56).
5 mg tablets: Brown, round biconvex, film-coated, debossed with “5” on one side and “EF” on the other side.
Bottle of 28-tablets with child resistant closure (NDC 59676-050-28).

Store at 20° C. to 25° C. (68° F. to 77° F.); excursions permitted between 15° C. to 30° C. (59° F. to 86° F.) [see USP Controlled Room Temperature].

### 17 Patient Counseling Information

Advise the patient to read the FDA-approved patient labeling (Patient Information).

### FGFR Genetic Alterations

Advise patients that evidence of a susceptible FGFR3 mutation or gene fusion within the tumor specimen is necessary to identify patients for whom treatment is indicated [see Dosage and Administration (2.1)].

### Ocular Disorders

Advise patients to contact their healthcare provider if they experience any visual changes [see Warnings and Precautions (5.1)]. In order to prevent or treat dry eyes, advise patients to use artificial tear substitutes, hydrating or lubricating eye gels or ointments frequently, at least every 2 hours during waking hours [see Dosage and Administration (2.3)].

### Skin, Mucous or Nail Disorders

Advise patients to contact their healthcare provider if they experience progressive or intolerable skin, mucous or nail disorders [see Adverse Reactions (6.1)].

### Hyperphosphatemia and Soft Tissue Mineralization

Inform patients that BALVERSA may cause hyperphosphatemia and soft tissue mineralization. Advise patients to immediately inform their healthcare provider of painful skin lesions or any symptoms related to acute change in phosphate levels such as muscle cramps, numbness, or tingling around the mouth [see Warnings and Precautions (5.2)].

Advise patients that their healthcare provider will assess their serum phosphate level between 14 and 21 days of initiating treatment and will adjust the dose if needed [see Warnings and Precautions (5.2)]. Advise patients to restrict phosphate intake to 600-800 mg daily. During this initial phosphate-assessment period, advise patients to avoid concomitant use with agents that can alter serum phosphate levels. Advise patients that, after the initial phosphate assessment period, monthly phosphate level monitoring for hyperphosphatemia should be performed during treatment with BALVERSA [see Drug Interactions (7.1)].

### Drug Interactions

Advise patients to inform their healthcare providers of all concomitant medications, including prescription medicines, over-the-counter drugs, and herbal products [see Drug Interactions (7.1, 7.2)].

### Dosing Instructions

Instruct patients to swallow the tablets whole once daily with or without food. If vomiting occurs any time after taking BALVERSA, advise patients to take the next dose the next day [see Dosage and Administration (2.2)].

### Missed Dose

If a dose is missed, advise patients to take the missed dose as soon as possible. Resume the regular daily dose schedule for BALVERSA the next day. Extra tablets should not be taken to make up for the missed dose [see Dosage and Administration (2.3)].

### Embryo-Fetal Toxicity

Advise pregnant women and females of reproductive potential of the potential risk to the fetus. Advise females to inform their healthcare providers of a known or suspected pregnancy [see Warnings and Precautions (5.3) and Use in Specific Populations (8.1)].

Advise female patients of reproductive potential to use effective contraception during treatment and for one month after the last dose of BALVERSA. Advise male patients with female partners of reproductive potential to use effective contraception during treatment and for one month after the last dose of BALVERSA [see Use in Specific Populations (8.3)].

### Lactation

Advise females not to breastfeed during treatment with BALVERSA and for one month after the last dose [see Use in Specific Populations (8.2)].

### Infertility

Advise females of reproductive potential that BALVERSA may impair fertility [see Use in Specific Populations (8.3)].
Product of Switzerland
Manufactured for:
Janssen Products, LP
Horsham, PA 19044, USA
Under license from Astex Therapeutics Limited.
For patent information: www.janssenpatents.com
©2019 Janssen Pharmaceutical Companies

### Patient Information

### BALVERSA® (Bal-VER-Sah)

### (Erdafitinib) Tablets

### What is BALVERSA?

BALVERSA is a prescription medicine used to treat adults with bladder cancer (urothelial cancer) that has spread or cannot be removed by surgery:
which has a certain type of abnormal FGFR gene, and
who have tried at least one other medicine by mouth or injection (systemic therapy) that did not work or is no longer working.

Your healthcare provider will test your cancer for certain types of abnormal FGFR genes and make sure that BALVERSA is right for you.

BALVERSA is not recommended for the treatment of people who are eligible for and have not received prior PD-1 or PD-L1 inhibitor therapy.

It is not known if BALVERSA is safe and effective in children.

Before Taking BALVERSA Tell Your Healthcare Provider about all of Your Medical Conditions, Including if You:
have vision or eye problems.
are pregnant or plan to become pregnant. BALVERSA can harm your unborn baby. You should not become pregnant during treatment with BALVERSA.

### Females Who can Become Pregnant:

Your healthcare provider may do a pregnancy test before you start treatment with BALVERSA.
You should use effective birth control during treatment and for 1 month after the last dose of BALVERSA. Talk to your healthcare provider about birth control methods that may be right for you.
Tell your healthcare provider right away if you become pregnant or think you may be pregnant.
   Males with Female Partners Who can Become Pregnant:
You should use effective birth control when sexually active during treatment with BALVERSA and for 1 month after the last dose.
are breastfeeding or plan to breastfeed. Do not breastfeed during treatment and for 1 month after the last dose of BALVERSA.

Tell your healthcare provider about all the medicines you take, including prescription and over-the-counter medicines, vitamins, and herbal supplements.

How should I Take BALVERSA?
Take BALVERSA exactly as your healthcare provider tells you.
Take BALVERSA 1 time each day.
Swallow BALVERSA tablets whole with or without food.
Your healthcare provider may change your dose of BALVERSA, temporarily stop or completely stop treatment if you get certain side effects.
If you miss a dose of BALVERSA, take the missed dose as soon as possible on the same day. Take your regular dose of BALVERSA the next day. Do not take more BALVERSA than prescribed to make up for the missed dose.
If you vomit after taking BALVERSA, do not take another BALVERSA tablet. Take your regular dose of BALVERSA the next day.

### What are the Possible Side Effects of BALVERSA?

### BALVERSA May Cause Serious Side Effects, Including:

Eye problems. Eye problems are common with BALVERSA but can also be serious. Eye problems include dry or inflamed eyes, inflamed cornea (front part of the eye) and disorders of the retina, an internal part of the eye. Tell your healthcare provider right away if you develop blurred vision, loss of vision or other visual changes. You should use artificial tear substitutes, hydrating or lubricating eye gels or ointments at least every 2 hours during waking hours to help prevent dry eyes. During treatment with BALVERSA, your healthcare provider will send you to see an eye specialist.
High phosphate levels in the blood (hyperphosphatemia). Hyperphosphatemia is common with BALVERSA but can also be serious. High levels of phosphate in your blood may lead to build-up of minerals such as calcium in different tissues in your body. Your healthcare provider will check your blood phosphate level between 14 and 21 days after starting treatment with BALVERSA, and then monthly.
   Your healthcare provider may prescribe changes in your diet or phosphate lowering therapy, or change or stop treatment with BALVERSA if needed.
   Tell your healthcare provider right away if you develop painful skin lesions, any muscle cramps, or numbness or tingling around your mouth.

The most common side effects of BALVERSA include:
Nails separate from the bed or poor formation of the nail
mouth sores
diarrhea
increased level of creatinine in the blood
increased level of the enzyme alkaline phosphatase in the blood
change in liver function
decreased red blood cells (anemia)
decreased salt (sodium) levels in the blood
tiredness
dry mouth
dry skin
decreased phosphate in the blood
decreased appetite
change in sense of taste
constipation
increased level of calcium in the blood
dry eye
redness, swelling, peeling or tenderness, mainly on the hands or feet (hand-foot syndrome
increased level of potassium in the blood
hair loss
fluid buildup behind the retina in your eye

Tell your healthcare provider right away if you develop any nail or skin problems including nails separating from the nail bed, nail pain, nail bleeding, breaking of the nails, color or texture changes in your nails, infected skin around the nail, an itchy skin rash, dry skin, or cracks in the skin.

BALVERSA may affect fertility in females who are able to become pregnant. Talk to your healthcare provider if this is a concern for you.

These are not all of the possible side effects of BALVERSA. Call your healthcare provider for medical advice about side effects. You may report side effects to FDA at 1-800-FDA-1088.

How should I Store BALVERSA?

Store BALVERSA tablets at room temperature between 68° F. to 77° F. (20° C. to 25° C.).

### Keep BALVERSA and all Medicines Out of the Reach of Children.

General Information about the Safe and Effective Use of BALVERSA.

Medicines are sometimes prescribed for purposes other than those listed in Patient Information leaflets. Do not use BALVERSA for a condition for which it was not prescribed. Do not give BALVERSA to other people, even if they have the same symptoms that you have. It may harm them. If you would like more information, talk with your healthcare provider. You can ask your pharmacist or healthcare provider for information about BALVERSA that is written for healthcare professionals.

### What are the Ingredients in BALVERSA?

Active ingredient: erdafitinib

### Inactive Ingredients:

Tablet Core: Croscarmellose sodium, Magnesium stearate (from vegetable source), Mannitol, Meglumine, and Microcrystalline Cellulose.

Film Coating (Opadry amb II): Glycerol monocaprylocaprate Type I, Polyvinyl alcohol partially hydrolyzed, Sodium lauryl sulfate, Talc, Titanium dioxide, Iron oxide yellow, Iron oxide red (for the orange and brown tablets only), Ferrosoferric oxide/iron oxide black (for the brown tablets only).

The examples and embodiments described herein are for illustrative purposes only and various modifications or changes suggested to persons skilled in the art are to be included within the spirit and purview of this application and scope of the appended claims.

The invention will now be described with reference to the following numbered clauses. For the avoidance of doubt, these numbered clauses do not limit the scope of the invention. Modifications may be made whilst remaining within the scope and spirit of the invention.
1. A method of treating locally advanced or metastatic urothelial carcinoma (mUC) harboring one or more fibroblast growth factor receptor (FGFR) genetic alterations, comprising administering to a subject in need thereof, in particular a patient with locally advanced or metastatic urothelial carcinoma, more in particular an adult patient with locally advanced or metastatic urothelial carcinoma, a therapeutically effective amount of erdafitinib, wherein the subject, in particular the patient, more in particular the adult patient, received at least one line of systemic treatment containing an anti-PD-(L)1 agent in the locally advanced or metastatic treatment.
2. A method of treating locally advanced or metastatic urothelial carcinoma (mUC), harboring one or more fibroblast growth factor receptor (FGFR) genetic alterations, comprising administering to a subject in need thereof, in particular a patient with locally advanced or metastatic urothelial carcinoma, more in particular an adult patient with locally advanced or metastatic urothelial carcinoma, a therapeutically effective amount of erdafitinib, wherein the subject, in particular the patient, more in particular the adult patient, progressed on or after ≥1 prior treatment that included an anti-PD-(L)1 agent.
3. A method of treating locally advanced or metastatic urothelial carcinoma (mUC) harboring one or more FGFR genetic alterations, comprising administering to a subject in need thereof, in particular a patient with locally advanced or metastatic urothelial carcinoma, more in particular an adult patient with locally advanced or metastatic urothelial carcinoma, a therapeutically effective amount of erdafitinib, wherein the subject, in particular the patient, more in particular the adult patient, progressed during or following at least one line of prior treatment with an anti-PD-(L)1 agent including within 12 months of neoadjuvant or adjuvant therapy.
4. A method for the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who received at least one line of systemic treatment containing an anti-PD-(L)1 agent in the locally advanced or metastatic treatment setting, comprising administering to the subject, in particular the patient, more in particular the adult patient, a therapeutically effective amount of erdafitinib.
5. A method for the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who progressed on or after ≥1 prior treatment that included an anti-PD-(L)1 agent, comprising administering to the subject, in particular the patient, more in particular the adult patient, a therapeutically effective amount of erdafitinib.
6. A method for the treatment of a subject in need thereof, in particular a patient with locally advanced or metastatic urothelial carcinoma, more in particular an adult patient with locally advanced or metastatic urothelial carcinoma, that has
   one or more FGFR genetic alterations and
   progressed during or following at least one line of prior treatment with an anti-PD-(L)1 agent including within 12 months of neoadjuvant or adjuvant therapy, comprising administering to the subject, in particular the patient, more in particular the adult patient, a therapeutically effective amount of erdafitinib.
7. A method of treating locally advanced or metastatic urothelial carcinoma (mUC) comprising
(a) evaluating a biological sample from a subject in need of the treatment, in particular a patient with locally advanced or metastatic urothelial carcinoma, more in particular an adult patient with locally advanced or metastatic urothelial carcinoma, and wherein the subject, in particular the patient, more in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample.
8. A method for the treatment of a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the subject, in particular the patient, more in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample.
9. Use of erdafitinib for the manufacture of a medicament for the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, received at least one line of systemic treatment containing an anti-PD-(L)1 agent in the locally advanced or metastatic treatment.
10. Use of erdafitinib for the manufacture of a medicament for the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, progressed on or after ≥1 prior treatment that included an anti-PD-(L)1 agent.
11. Use of erdafitinib for the manufacture of a medicament for the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, progressed during or following at least one line of prior treatment with an anti-PD-(L)1 agent including within 12 months of neoadjuvant or adjuvant therapy.
12. Use of erdafitinib for the manufacture of a medicament for the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who has received at least one line of systemic treatment containing an anti-PD-(L)1 agent in the locally advanced or metastatic treatment setting, wherein erdafitinib is administered or is to be administered in a therapeutically effective amount.
13. Use of erdafitinib for the manufacture of a medicament for the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who progressed on or after ≥1 prior treatment that included an anti-PD-(L)1 agent, wherein erdafitinib is administered or is to be administered in a therapeutically effective amount.
14. Use of erdafitinib for the manufacture of a medicament for the treatment of a subject in need thereof, in particular a patient with locally advanced or metastatic urothelial carcinoma, more in particular an adult patient with locally advanced or metastatic urothelial carcinoma, that has
   one or more FGFR genetic alterations and
   progressed during or following at least one line of prior treatment with an anti-PD-(L)1 agent including within 12 months of neoadjuvant or adjuvant therapy, wherein erdafitinib is administered or is to be administered in a therapeutically effective amount.
15. Use of erdafitinib for the manufacture of a medicament for the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the subject, in particular the patient, more in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample.
16. Use of erdafitinib for the manufacture of a medicament for the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the subject, in particular the patient, more in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample.
17. Erdafitinib for use in the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, received at least one line of systemic treatment containing an anti-PD-(L)1 agent in the locally advanced or metastatic treatment.
18. Erdafitinib for use in the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, progressed on or after ≥1 prior treatment that included an anti-PD-(L)1 agent.
19. Erdafitinib for use in the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, progressed during or following at least one line of prior treatment with an anti-PD-(L)1 agent including within 12 months of neoadjuvant or adjuvant therapy.
20. Erdafitinib for use in the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who received at least one line of systemic treatment containing an anti-PD-(L)1 agent in the locally advanced or metastatic treatment setting, wherein erdafitinib is administered or is to be administered in a therapeutically effective amount.
21. Erdafitinib for use in the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who progressed on or after ≥1 prior treatment that included an anti-PD-(L)1 agent, wherein erdafitinib is administered or is to be administered in a therapeutically effective amount.
22. Erdafitinib for use in the treatment of a subject in need thereof, in particular a patient with locally advanced or metastatic urothelial carcinoma, more in particular an adult patient with locally advanced or metastatic urothelial carcinoma that has
   one or more FGFR genetic alterations and
   progressed during or following at least one line of prior treatment with an anti-PD-(L)1 agent including within 12 months of neoadjuvant or adjuvant therapy, wherein erdafitinib is administered or is to be administered in a therapeutically effective amount.
23. Erdafitinib for use in the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the subject, in particular the patient, more in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample.
24. Erdafitinib for use in the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the subject, in particular the patient, more in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample.
25. A method of improving overall survival in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib.
26. A method of improving overall survival in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient a therapeutically effective amount of erdafitinib.
27. A method of improving overall survival in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient a therapeutically effective amount of erdafitinib.
28. A method of improving overall survival in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
29. A method of improving overall survival in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
30. Use of erdafitinib for the manufacture of a medicament for improving overall survival in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.
31. Use of erdafitinib for the manufacture of a medicament for improving overall survival in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.
32. Use of erdafitinib for the manufacture of a medicament for improving overall survival in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.
33. Use of erdafitinib for the manufacture of a medicament for improving overall survival in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
34. Use of erdafitinib for the manufacture of a medicament for improving overall survival in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
35. Erdafitinib for use in improving overall survival in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.
36. Erdafitinib for use in improving overall survival in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.
37. Erdafitinib for use in improving overall survival in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.
38. Erdafitinib for use in improving overall survival in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
39. Erdafitinib for use in improving overall survival in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
40. A method of reducing risk of death in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib.
41. A method of reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient a therapeutically effective amount of erdafitinib.
42. A method of reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient a therapeutically effective amount of erdafitinib.
43. A method of reducing risk of death in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
44. A method of reducing risk of death in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
45. Use of erdafitinib for the manufacture of a medicament for reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.
46. Use of erdafitinib for the manufacture of a medicament for reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.
47. Use of erdafitinib for the manufacture of a medicament for reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.
48. Use of erdafitinib for the manufacture of a medicament for reducing risk of death in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
49. Use of erdafitinib for the manufacture of a medicament for reducing risk of death in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
50. Erdafitinib for use in reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.
51. Erdafitinib for use in reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.
52. Erdafitinib for use in reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.
53. Erdafitinib for use in reducing risk of death in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
54. Erdafitinib for use in reducing risk of death in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
55. A method of improving median PFS in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib.
56. A method of improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient a therapeutically effective amount of erdafitinib.
57. A method of improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient a therapeutically effective amount of erdafitinib.
58. A method of improving median PFS in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
59. A method of improving median PFS in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
60. Use of erdafitinib for the manufacture of a medicament for improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.
61. Use of erdafitinib for the manufacture of a medicament for improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.
62. Use of erdafitinib for the manufacture of a medicament for improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.
63. Use of erdafitinib for the manufacture of a medicament for improving median PFS in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
64. Use of erdafitinib for the manufacture of a medicament for improving median PFS in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
65. Erdafitinib for use in improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.
66. Erdafitinib for use in improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.
67. Erdafitinib for use in improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.
68. Erdafitinib for use in improving median PFS in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
69. Erdafitinib for use in improving median PFS in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
70. A method of reducing risk of progression or death in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib.
71. A method of reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient a therapeutically effective amount of erdafitinib.
72. A method of reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient a therapeutically effective amount of erdafitinib.
73. A method of reducing risk of progression or death in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
74. A method of reducing risk of progression or death in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
75. Use of erdafitinib for the manufacture of a medicament for reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.
76. Use of erdafitinib for the manufacture of a medicament for reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.
77. Use of erdafitinib for the manufacture of a medicament for reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.
78. Use of erdafitinib for the manufacture of a medicament for reducing risk of progression or death in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
79. Use of erdafitinib for the manufacture of a medicament for reducing risk of progression or death in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
80. Erdafitinib for use in reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.
81. Erdafitinib for use in reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.
82. Erdafitinib for use in reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.
83. Erdafitinib for use in reducing risk of progression or death in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
84. Erdafitinib for use in reducing risk of progression or death in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
85. A method of improving ORR in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib.
86. A method of improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient a therapeutically effective amount of erdafitinib.
87. A method of improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient a therapeutically effective amount of erdafitinib.
88. A method of improving ORR in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
89. A method of improving ORR in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
90. Use of erdafitinib for the manufacture of a medicament for improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.
91. Use of erdafitinib for the manufacture of a medicament for improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.
92. Use of erdafitinib for the manufacture of a medicament for improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.
93. Use of erdafitinib for the manufacture of a medicament for improving ORR in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
94. Use of erdafitinib for the manufacture of a medicament for improving ORR in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
95. Erdafitinib for use in improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, and wherein erdafitinib is administered or is to be administered in a therapeutically effective amount.
96. Erdafitinib for use in improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, and wherein erdafitinib is administered or is to be administered in a therapeutically effective amount.
97. Erdafitinib for use in improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, and wherein erdafitinib is administered or is to be administered in a therapeutically effective amount.
98. Erdafitinib for use in improving ORR in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
99. Erdafitinib for use in improving ORR in a patient with locally advanced or metastatic urothelial carcinoma (mUC), in particular an adult patient with locally advanced or metastatic urothelial carcinoma, comprising
(a) evaluating a biological sample from the patient, in particular the adult patient, and wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
100. The method, use or for use according to any one of claims 25 to 39 wherein the improvement of overall survival is a median overall survival of about 12.1 months.
101. The method, use or for use according to any one of claims 25 to 39 wherein the improvement of overall survival is versus chemotherapy.
102. The method, use or for use according to any one of claims 25 to 39 wherein the improvement of overall survival is a median overall survival of about 12.1 months versus a median overall survival of about 7.8 months for chemotherapy.
103. The method, use or for use according to claim 101 or 102 wherein the chemotherapy is docetaxel or vinflunine.
104. The method, use or for use according to any one of claims 40 to 54 wherein the reduced risk of death is a reduced risk of death by about 36%.
105. The method, use or for use according to any one of claims 40 to 54 wherein the reduced risk of death is versus chemotherapy.
106. The method, use or for use according to any one of claims 40 to 54 wherein the reduced risk of death is a reduced risk of death by about 36% versus chemotherapy.
107. The method, use or for use according to claim 105 or 106 wherein the chemotherapy is docetaxel or vinflunine.
108. The method, use or for use according to any one of claims 55 to 69 wherein the improvement of PFS is a median PFS of about 5.6 months.
109. The method, use or for use according to any one of claims 55 to 69 wherein the improvement of PFS is versus chemotherapy.
110. The method, use or for use according to any one of claims 55 to 69 wherein the improvement of PFS is a median PFS of about 5.6 months versus a median PFS of about 2.7 months for chemotherapy.
111. The method, use or for use according to claim 109 or 110 wherein the chemotherapy is docetaxel or vinflunine.
112. The method, use or for use according to any one of claims 70 to 84 wherein the reduced risk of progression or death is a reduced risk of progression or death by about 42%.
113. The method, use or for use according to any one of claims 70 to 84 wherein the reduced risk of progression or death is versus chemotherapy.
114. The method, use or for use according to any one of claims 70 to 84 wherein the reduced risk of progression or death is a reduced risk of progression or death of about 42% versus chemotherapy.
115. The method, use or for use according to claim 113 or 114 wherein the chemotherapy is docetaxel or vinflunine.
116. The method, use or for use according to any one of claims 85 to 99 wherein the improvement of ORR is a ORR of about 45.6%.
117. The method, use or for use according to any one of claims 85 to 99 wherein the improvement of ORR is versus chemotherapy.
118. The method, use or for use according to any one of claims 85 to 99 wherein the improvement of ORR is a ORR of about 45.6% versus a ORR of 11.5% for chemotherapy.
119. The method, use or for use according to claim 117 or 118 wherein the chemotherapy is docetaxel or vinflunine.
120. The method, use or for use according to any one of the preceding claims wherein the one or more FGFR genetic alterations are selected from a FGFR fusion and/or mutation.
121. The method, use or for use according to any one of the preceding claims wherein the one or more FGFR genetic alterations are selected from a FGFR3 mutation and/or a FGFR2 fusion and/or a FGFR 3 fusions.
122. The method, use or for use according to any one of the preceding claims wherein the one or more FGFR genetic alterations are selected from FGFR3-TACC3, in particular FGFR3-TACC3 v1 and FGFR3-TACC3 v3; FGFR3-BAIAP2L1; FGFR2-BICC1; FGFR2-CASP7; FGFR3 R248C; FGFR3 S249C; FGFR3 G370C; and FGFR3 Y373C. I 123. The method, use or for use according to any one of claims 1 to 119 wherein the one or more FGFR genetic alterations are selected from a FGFR3 mutation and/or a FGFR3 fusion.
124. The method, use or for use according to claim 123 wherein the one or more FGFR genetic alterations are selected from FGFR3-TACC3, in particular FGFR3-TACC3 v1 and FGFR3-TACC3 v3; FGFR3-BAIAP2L1; FGFR3 R248C; FGFR3 S249C; FGFR3 G370C; and FGFR3 Y373C.
125. The method, use or for use according to any one of claims 1 to 119 wherein the one or more FGFR genetic alterations comprise or consists of FGFR3 S249C.
126. The method, use or for use according to any one of claims 1 to 119 wherein the one or more FGFR genetic alterations comprise or consists of FGFR3 Y373C.
127. The method, use or for use according to any one of claims 1 to 119 wherein the one or more FGFR genetic alterations comprise or consists of FGFR3 G370C.
128. The method, use or for use according to any one of claims 1 to 119 wherein the one or more FGFR genetic alterations comprise or consists of FGFR3 R248C.
129. The method, use or for use according to any one of claims 1 to 119 wherein the one or more FGFR genetic alterations comprise or consists of FGFR3-TACC3 v1.
130. The method, use or for use according to any one of claims 1 to 119 wherein the one or more FGFR genetic alterations comprise or consists of FGFR3-TACC3 v3.
131. The method, use or for use according to any one of claims 1 to 119 wherein the one or more FGFR genetic alterations comprise or consists of FGFR3-BAIAP2L1.
132. The method, use or for use according to any one of claims 1 to 119 wherein the one or more FGFR genetic alterations comprise or consists of FGFR2-BICC1.
133. The method, use or for use according to any one of claims 1 to 119 wherein the one or more FGFR genetic alterations comprise or consists of FGFR2-CASP7.
134. The method, use or for use according to any one of the preceding claims wherein the therapeutically effective amount of erdafitinib is 8 mg daily, in particular 8 mg once daily.
135. The method, use or for use according to any one of claims 1 to 133 wherein the therapeutically effective amount of erdafitinib is 9 mg daily, in particular 9 mg once daily.
136. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on any of day 14 to 21 after initiating erdafitinib treatment.
137. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on day 14 of erdafitinib treatment.
138. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on day 15 of erdafitinib treatment.
139. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on day 16 of erdafitinib treatment.
140. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on day 17 of erdafitinib treatment
141. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on day 18 of erdafitinib treatment.
142. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on day 19 of erdafitinib treatment.
143. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on day 20 of erdafitinib treatment.
144. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on day 21 of erdafitinib treatment.
145. The method, use or for use according to any one of claims 136 to 144 wherein the erdafitinib dose is uptitrated to 9 mg daily, in particular once daily, if serum phosphate level is <5.5 mg/dL.
146. The method, use or for use according to any one of claims 136 to 144 wherein the erdafitinib dose is uptitrated to 9 mg daily, in particular once daily, if serum phosphate level is <7 mg/dL.
147. The method, use or for use according to any one of claims 136 to 144 wherein the erdafitinib dose is uptitrated to 9 mg daily, in particular once daily, if serum phosphate level is <9 mg/dL, in particular if the serum phosphate level assessed 14 to 21 days after initiating erdafitinib treatment is ranging from and including 7.00 to 8.99 mg/dL while concurrently initiating treatment with a phosphate binder such as for example sevelamer.
148. The method, use or for use according to any one of claims 136 to 147 wherein the erdafitinib dose is uptitrated to 9 mg daily, in particular once daily when there is no drug-related toxicity.
149. The method, use or for use according to any one of claims 136 to 147 wherein the erdafitinib dose is uptitrated to 9 mg daily, in particular once daily when there are no ocular disorders or Grade 2 or greater adverse reactions.
150. The method, use or for use according to any one of claims 134, 136 to 149 wherein the 8 mg dose of erdafitinib is administered or is to be administered as 2 formulations, in particular 2 tablets, each comprising 4 mg of erdafitinib.
151. The method, use or for use according to any one of claims 134, 136 to 149 wherein the 8 mg dose of erdafitinib is administered or is to be administered as 2 formulations, in particular 2 tablets, one comprising 3 mg of erdafitinib and one comprising 5 mg erdafitinib.
151. The method, use or for use according to any one of claims 135 to 149 wherein the 9 mg dose of erdafitinib is administered or is to be administered as 3 formulations, in particular 3 tablets, each comprising 3 mg of erdafitinib.
152. The method, use or for use according to any one of claims 135 to 149 wherein the 9 mg dose of erdafitinib is administered or is to be administered as 2 formulations, in particular 2 tablets, one comprising 4 mg of erdafitinib and one comprising 5 mg erdafitinib.
1. A method of treating locally advanced or metastatic urothelial carcinoma (mUC) harboring one or more fibroblast growth factor receptor (FGFR) genetic alterations, comprising administering to a subject in need thereof, in particular a patient with locally advanced or metastatic urothelial carcinoma, more in particular an adult patient with locally advanced or metastatic urothelial carcinoma, a therapeutically effective amount of erdafitinib, wherein the subject, in particular the patient, more in particular the adult patient, received at least one line of systemic treatment containing an anti-PD-(L)1 agent in the locally advanced or metastatic treatment.
2. A method of treating locally advanced or metastatic urothelial carcinoma (mUC), harboring one or more fibroblast growth factor receptor (FGFR) genetic alterations, comprising administering to a subject in need thereof, in particular a patient with locally advanced or metastatic urothelial carcinoma, more in particular an adult patient with locally advanced or metastatic urothelial carcinoma, a therapeutically effective amount of erdafitinib, wherein the subject, in particular the patient, more in particular the adult patient, progressed on or after ≥1 prior treatment that included an anti-PD-(L)1 agent.
3. A method of treating locally advanced or metastatic urothelial carcinoma (mUC) harboring one or more FGFR genetic alterations, comprising administering to a subject in need thereof, in particular a patient with locally advanced or metastatic urothelial carcinoma, more in particular an adult patient with locally advanced or metastatic urothelial carcinoma, a therapeutically effective amount of erdafitinib, wherein the subject, in particular the patient, more in particular the adult patient, progressed during or following at least one line of prior treatment with an anti-PD-(L)1 agent including within 12 months of neoadjuvant or adjuvant therapy.
4. A method for the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who received at least one line of systemic treatment containing an anti-PD-(L)1 agent in the locally advanced or metastatic treatment setting, comprising administering to the subject, in particular the patient, more in particular the adult patient, a therapeutically effective amount of erdafitinib.
5. A method for the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who progressed on or after ≥1 prior treatment that included an anti-PD-(L)1 agent, comprising administering to the subject, in particular the patient, more in particular the adult patient, a therapeutically effective amount of erdafitinib.
6. A method for the treatment of a subject in need thereof, in particular a patient with locally advanced or metastatic urothelial carcinoma, more in particular an adult patient with locally advanced or metastatic urothelial carcinoma, that has
   one or more FGFR genetic alterations and
   progressed during or following at least one line of prior treatment with an anti-PD-(L)1 agent including within 12 months of neoadjuvant or adjuvant therapy, comprising administering to the subject, in particular the patient, more in particular the adult patient, a therapeutically effective amount of erdafitinib.
7. A method of treating locally advanced or metastatic urothelial carcinoma (mUC) comprising
(a) evaluating a biological sample from a subject in need of the treatment, in particular a patient with locally advanced or metastatic urothelial carcinoma, more in particular an adult patient with locally advanced or metastatic urothelial carcinoma, and wherein the subject, in particular the patient, more in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample.
8. A method for the treatment of a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the subject, in particular the patient, more in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample.
9. Use of erdafitinib for the manufacture of a medicament for the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, received at least one line of systemic treatment containing an anti-PD-(L)1 agent in the locally advanced or metastatic treatment.
10. Use of erdafitinib for the manufacture of a medicament for the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, progressed on or after ≥1 prior treatment that included an anti-PD-(L)1 agent.
11. Use of erdafitinib for the manufacture of a medicament for the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, progressed during or following at least one line of prior treatment with an anti-PD-(L)1 agent including within 12 months of neoadjuvant or adjuvant therapy.
12. Use of erdafitinib for the manufacture of a medicament for the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who has received at least one line of systemic treatment containing an anti-PD-(L)1 agent in the locally advanced or metastatic treatment setting, wherein erdafitinib is administered or is to be administered in a therapeutically effective amount.
13. Use of erdafitinib for the manufacture of a medicament for the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who progressed on or after ≥1 prior treatment that included an anti-PD-(L)1 agent, wherein erdafitinib is administered or is to be administered in a therapeutically effective amount.
14. Use of erdafitinib for the manufacture of a medicament for the treatment of a subject in need thereof, in particular a patient with locally advanced or metastatic urothelial carcinoma, more in particular an adult patient with locally advanced or metastatic urothelial carcinoma, that has
   one or more FGFR genetic alterations and
   progressed during or following at least one line of prior treatment with an anti-PD-(L)1 agent including within 12 months of neoadjuvant or adjuvant therapy, wherein erdafitinib is administered or is to be administered in a therapeutically effective amount.
15. Use of erdafitinib for the manufacture of a medicament for the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the subject, in particular the patient, more in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample.
16. Use of erdafitinib for the manufacture of a medicament for the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the subject, in particular the patient, more in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample.
17. Erdafitinib for use in the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, received at least one line of systemic treatment containing an anti-PD-(L)1 agent in the locally advanced or metastatic treatment.
18. Erdafitinib for use in the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, progressed on or after ≥1 prior treatment that included an anti-PD-(L)1 agent.
19. Erdafitinib for use in the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, wherein erdafitinib is administered or is to be administered in a therapeutically effective amount, and wherein the subject, in particular the patient, more in particular the adult patient, progressed during or following at least one line of prior treatment with an anti-PD-(L)1 agent including within 12 months of neoadjuvant or adjuvant therapy.
20. Erdafitinib for use in the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who received at least one line of systemic treatment containing an anti-PD-(L)1 agent in the locally advanced or metastatic treatment setting, wherein erdafitinib is administered or is to be administered in a therapeutically effective amount.
21. Erdafitinib for use in the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who progressed on or after ≥1 prior treatment that included an anti-PD-(L)1 agent, wherein erdafitinib is administered or is to be administered in a therapeutically effective amount.
22. Erdafitinib for use in the treatment of a subject in need thereof, in particular a patient with locally advanced or metastatic urothelial carcinoma, more in particular an adult patient with locally advanced or metastatic urothelial carcinoma that has
   one or more FGFR genetic alterations and
   progressed during or following at least one line of prior treatment with an anti-PD-(L)1 agent including within 12 months of neoadjuvant or adjuvant therapy, wherein erdafitinib is administered or is to be administered in a therapeutically effective amount.
23. Erdafitinib for use in the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the subject, in particular the patient, more in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample.
24. Erdafitinib for use in the treatment of locally advanced or metastatic urothelial carcinoma (mUC), harboring one or more fibroblast growth factor receptor (FGFR) genetic alterations, in a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the subject, in particular the patient, more in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample.
25. A method of improving overall survival in a population of patients with locally advanced or metastatic urothelial carcinoma harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, received at least one line of systemic treatment containing an anti-PD-(L)1 agent in the locally advanced or metastatic treatment, comprising administering to said population of patients, in particular said population of adult patients, a therapeutically effective amount of erdafitinib.
26. A method of improving overall survival in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed on or after ≥1 prior treatment that included an anti-PD-(L)1 agent, comprising administering to said population of patients, in particular said population of adult patients a therapeutically effective amount of erdafitinib.
27. A method of improving overall survival in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed during or following at least one line of prior treatment with an anti-PD-(L)1 agent including within 12 months of neoadjuvant or adjuvant therapy, comprising administering to said population of patients, in particular said population of adult patients a therapeutically effective amount of erdafitinib.
28. A method of improving overall survival in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, wherein the population of patients, in particular the population of adult patients, received at least one line of systemic treatment containing an anti-PD-(L)1 agent in the locally advanced or metastatic treatment, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
29. A method of improving overall survival in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, wherein the population of patients, in particular the population of adult patients, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
30. Use of erdafitinib for the manufacture of a medicament for improving overall survival in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, received at least one line of systemic treatment containing an anti-PD-(L)1 agent in the locally advanced or metastatic treatment, and wherein erdafitinib is administered or is to be administered in a therapeutically effective amount.
31. Use of erdafitinib for the manufacture of a medicament for improving overall survival in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed on or after ≥1 prior treatment that included an anti-PD-(L)1 agent, and wherein erdafitinib is administered or is to be administered in a therapeutically effective amount.
32. Use of erdafitinib for the manufacture of a medicament for improving overall survival in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed during or following at least one line of prior treatment with an anti-PD-(L)1 agent including within 12 months of neoadjuvant or adjuvant therapy, and wherein erdafitinib is administered or is to be administered in a therapeutically effective amount.
33. Use of erdafitinib for the manufacture of a medicament for improving overall survival in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, received at least one line of systemic treatment containing an anti-PD-(L)1 agent in the locally advanced or metastatic treatment, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
34. Use of erdafitinib for the manufacture of a medicament for improving overall survival in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
35. Erdafitinib for use in improving overall survival in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, received at least one line of systemic treatment containing an anti-PD-(L)1 agent in the locally advanced or metastatic treatment, and wherein erdafitinib is administered or is to be administered in a therapeutically effective amount.
36. Erdafitinib for use in improving overall survival in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed on or after ≥1 prior treatment that included an anti-PD-(L)1 agent, and wherein erdafitinib is administered or is to be administered in a therapeutically effective amount.
37. Erdafitinib for use in improving overall survival in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed during or following at least one line of prior treatment with an anti-PD-(L)1 agent including within 12 months of neoadjuvant or adjuvant therapy, and wherein erdafitinib is administered or is to be administered in a therapeutically effective amount.
38. Erdafitinib for use in improving overall survival in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, received at least one line of systemic treatment containing an anti-PD-(L)1 agent in the locally advanced or metastatic treatment, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
39. Erdafitinib for use in improving overall survival in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
40. A method of reducing risk of death in a population of patients with locally advanced or metastatic urothelial carcinoma harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, received at least one line of systemic treatment containing an anti-PD-(L)1 agent in the locally advanced or metastatic treatment, comprising administering to said population of patients, in particular said population of adult patients, a therapeutically effective amount of erdafitinib.
41. A method of reducing risk of death in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed on or after ≥1 prior treatment that included an anti-PD-(L)1 agent, comprising administering to said population of patients, in particular said population of adult patients, a therapeutically effective amount of erdafitinib.
42. A method of reducing risk of death in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed during or following at least one line of prior treatment with an anti-PD-(L)1 agent including within 12 months of neoadjuvant or adjuvant therapy, comprising administering to said population of patients, in particular said population of adult patients, a therapeutically effective amount of erdafitinib.
43. A method of reducing risk of death in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, wherein the population of patients, in particular the population of adult patients, received at least one line of systemic treatment containing an anti-PD-(L)1 agent in the locally advanced or metastatic treatment, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
44. A method of reducing risk of death in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, wherein the population of patients, in particular the population of adult patients, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
45. Use of erdafitinib for the manufacture of a medicament for reducing risk of death in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, received at least one line of systemic treatment containing an anti-PD-(L)1 agent in the locally advanced or metastatic treatment, and wherein erdafitinib is administered or is to be administered in a therapeutically effective amount.
46. Use of erdafitinib for the manufacture of a medicament for reducing risk of death in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed on or after ≥1 prior treatment that included an anti-PD-(L)1 agent, and wherein erdafitinib is administered or is to be administered in a therapeutically effective amount.
47. Use of erdafitinib for the manufacture of a medicament for reducing risk of death in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed during or following at least one line of prior treatment with an anti-PD-(L)1 agent including within 12 months of neoadjuvant or adjuvant therapy, and wherein erdafitinib is administered or is to be administered in a therapeutically effective amount.
48. Use of erdafitinib for the manufacture of a medicament for reducing risk of death in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, received at least one line of systemic treatment containing an anti-PD-(L)1 agent in the locally advanced or metastatic treatment, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
49. Use of erdafitinib for the manufacture of a medicament for reducing risk of death in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
50. Erdafitinib for use in reducing risk of death in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, received at least one line of systemic treatment containing an anti-PD-(L)1 agent in the locally advanced or metastatic treatment, and wherein erdafitinib is administered or is to be administered in a therapeutically effective amount.
51. Erdafitinib for use in reducing risk of death in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed on or after ≥1 prior treatment that included an anti-PD-(L)1 agent, and wherein erdafitinib is administered or is to be administered in a therapeutically effective amount.
52. Erdafitinib for use in reducing risk of death in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed during or following at least one line of prior treatment with an anti-PD-(L)1 agent including within 12 months of neoadjuvant or adjuvant therapy, and wherein erdafitinib is administered or is to be administered in a therapeutically effective amount.
53. Erdafitinib for use in reducing risk of death in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, received at least one line of systemic treatment containing an anti-PD-(L)1 agent in the locally advanced or metastatic treatment, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
54. Erdafitinib for use in reducing risk of death in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
55. A method of improving median PFS in a population of patients with locally advanced or metastatic urothelial carcinoma harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, received at least one line of systemic treatment containing an anti-PD-(L)1 agent in the locally advanced or metastatic treatment, comprising administering to said population of patients, in particular said population of adult patients, a therapeutically effective amount of erdafitinib.
56. A method of improving median PFS in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed on or after ≥1 prior treatment that included an anti-PD-(L)1 agent, comprising administering to said population of patients, in particular said population of adult patients a therapeutically effective amount of erdafitinib.
57. A method of improving median PFS in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed during or following at least one line of prior treatment with an anti-PD-(L)1 agent including within 12 months of neoadjuvant or adjuvant therapy, comprising administering to said population of patients, in particular said population of adult patients a therapeutically effective amount of erdafitinib.
58. A method of improving median PFS in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, wherein the population of patients, in particular the population of adult patients, received at least one line of systemic treatment containing an anti-PD-(L)1 agent in the locally advanced or metastatic treatment, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
59. A method of improving median PFS in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, wherein the population of patients, in particular the population of adult patients, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
60. Use of erdafitinib for the manufacture of a medicament for improving median PFS in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, received at least one line of systemic treatment containing an anti-PD-(L)1 agent in the locally advanced or metastatic treatment, and wherein erdafitinib is administered or is to be administered in a therapeutically effective amount.
61. Use of erdafitinib for the manufacture of a medicament for improving median PFS in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed on or after ≥1 prior treatment that included an anti-PD-(L)1 agent, and wherein erdafitinib is administered or is to be administered in a therapeutically effective amount.
62. Use of erdafitinib for the manufacture of a medicament for improving median PFS in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed during or following at least one line of prior treatment with an anti-PD-(L)1 agent including within 12 months of neoadjuvant or adjuvant therapy, and wherein erdafitinib is administered or is to be administered in a therapeutically effective amount.
63. Use of erdafitinib for the manufacture of a medicament for improving median PFS in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, received at least one line of systemic treatment containing an anti-PD-(L)1 agent in the locally advanced or metastatic treatment, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
64. Use of erdafitinib for the manufacture of a medicament for improving median PFS in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
65. Erdafitinib for use in improving median PFS in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, received at least one line of systemic treatment containing an anti-PD-(L)1 agent in the locally advanced or metastatic treatment, and wherein erdafitinib is administered or is to be administered in a therapeutically effective amount.
66. Erdafitinib for use in improving median PFS in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed on or after ≥1 prior treatment that included an anti-PD-(L)1 agent, and wherein erdafitinib is administered or is to be administered in a therapeutically effective amount.
67. Erdafitinib for use in improving median PFS in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed during or following at least one line of prior treatment with an anti-PD-(L)1 agent including within 12 months of neoadjuvant or adjuvant therapy, and wherein erdafitinib is administered or is to be administered in a therapeutically effective amount.
68. Erdafitinib for use in improving median PFS in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, received at least one line of systemic treatment containing an anti-PD-(L)1 agent in the locally advanced or metastatic treatment, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
69. Erdafitinib for use in improving median PFS in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
70. A method of reducing risk of progression or death in a population of patients with locally advanced or metastatic urothelial carcinoma harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, received at least one line of systemic treatment containing an anti-PD-(L)1 agent in the locally advanced or metastatic treatment, comprising administering to said population of patients, in particular said population of adult patients, a therapeutically effective amount of erdafitinib.
71. A method of reducing risk of progression or death in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed on or after ≥1 prior treatment that included an anti-PD-(L)1 agent, comprising administering to said population of patients, in particular said population of adult patients a therapeutically effective amount of erdafitinib.
72. A method of reducing risk of progression or death in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed during or following at least one line of prior treatment with an anti-PD-(L)1 agent including within 12 months of neoadjuvant or adjuvant therapy, comprising administering to said population of patients, in particular said population of adult patients a therapeutically effective amount of erdafitinib.
73. A method of reducing risk of progression or death in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, wherein the population of patients, in particular the population of adult patients, received at least one line of systemic treatment containing an anti-PD-(L)1 agent in the locally advanced or metastatic treatment, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
74. A method of reducing risk of progression or death in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, wherein the population of patients, in particular the population of adult patients, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
75. Use of erdafitinib for the manufacture of a medicament for reducing risk of progression or death in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, received at least one line of systemic treatment containing an anti-PD-(L)1 agent in the locally advanced or metastatic treatment, and wherein erdafitinib is administered or is to be administered in a therapeutically effective amount.
76. Use of erdafitinib for the manufacture of a medicament for reducing risk of progression or death in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed on or after ≥1 prior treatment that included an anti-PD-(L)1 agent, and wherein erdafitinib is administered or is to be administered in a therapeutically effective amount.
77. Use of erdafitinib for the manufacture of a medicament for reducing risk of progression or death in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed during or following at least one line of prior treatment with an anti-PD-(L)1 agent including within 12 months of neoadjuvant or adjuvant therapy, and wherein erdafitinib is administered or is to be administered in a therapeutically effective amount.
78. Use of erdafitinib for the manufacture of a medicament for reducing risk of progression or death in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, received at least one line of systemic treatment containing an anti-PD-(L)1 agent in the locally advanced or metastatic treatment, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
79. Use of erdafitinib for the manufacture of a medicament for reducing risk of progression or death in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
80. Erdafitinib for use in reducing risk of progression or death in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, received at least one line of systemic treatment containing an anti-PD-(L)1 agent in the locally advanced or metastatic treatment, and wherein erdafitinib is administered or is to be administered in a therapeutically effective amount.
81. Erdafitinib for use in reducing risk of progression or death in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed on or after ≥1 prior treatment that included an anti-PD-(L)1 agent, and wherein erdafitinib is administered or is to be administered in a therapeutically effective amount.
82. Erdafitinib for use in reducing risk of progression or death in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed during or following at least one line of prior treatment with an anti-PD-(L)1 agent including within 12 months of neoadjuvant or adjuvant therapy, and wherein erdafitinib is administered or is to be administered in a therapeutically effective amount.
83. Erdafitinib for use in reducing risk of progression or death in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, received at least one line of systemic treatment containing an anti-PD-(L)1 agent in the locally advanced or metastatic treatment, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
84. Erdafitinib for use in reducing risk of progression or death in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
85. A method of improving ORR in a population of patients with locally advanced or metastatic urothelial carcinoma harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, received at least one line of systemic treatment containing an anti-PD-(L)1 agent in the locally advanced or metastatic treatment, comprising administering to said population of patients, in particular said population of adult patients, a therapeutically effective amount of erdafitinib.
86. A method of improving ORR in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed on or after ≥1 prior treatment that included an anti-PD-(L)1 agent, comprising administering to said population of patients, in particular said population of adult patients a therapeutically effective amount of erdafitinib.
87. A method of improving ORR in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed during or following at least one line of prior treatment with an anti-PD-(L)1 agent including within 12 months of neoadjuvant or adjuvant therapy, comprising administering to said population of patients, in particular said population of adult patients a therapeutically effective amount of erdafitinib.
88. A method of improving ORR in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, wherein the population of patients, in particular the population of adult patients, received at least one line of systemic treatment containing an anti-PD-(L)1 agent in the locally advanced or metastatic treatment, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
89. A method of improving ORR in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, wherein the population of patients, in particular the population of adult patients, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
90. Use of erdafitinib for the manufacture of a medicament for improving ORR in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, received at least one line of systemic treatment containing an anti-PD-(L)1 agent in the locally advanced or metastatic treatment, and wherein erdafitinib is administered or is to be administered in a therapeutically effective amount.
91. Use of erdafitinib for the manufacture of a medicament for improving ORR in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed on or after ≥1 prior treatment that included an anti-PD-(L)1 agent, and wherein erdafitinib is administered or is to be administered in a therapeutically effective amount.
92. Use of erdafitinib for the manufacture of a medicament for improving ORR in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed during or following at least one line of prior treatment with an anti-PD-(L)1 agent including within 12 months of neoadjuvant or adjuvant therapy, and wherein erdafitinib is administered or is to be administered in a therapeutically effective amount.
93. Use of erdafitinib for the manufacture of a medicament for improving ORR in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, received at least one line of systemic treatment containing an anti-PD-(L)1 agent in the locally advanced or metastatic treatment, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
94. Use of erdafitinib for the manufacture of a medicament for improving ORR in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
95. Erdafitinib for use in improving ORR in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, received at least one line of systemic treatment containing an anti-PD-(L)1 agent in the locally advanced or metastatic treatment, and wherein erdafitinib is administered or is to be administered in a therapeutically effective amount.
96. Erdafitinib for use in improving ORR in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed on or after ≥1 prior treatment that included an anti-PD-(L)1 agent, and wherein erdafitinib is administered or is to be administered in a therapeutically effective amount.
97. Erdafitinib for use in improving ORR in a population of patients with locally advanced or metastatic urothelial carcinoma (mUC) harboring one or more fibroblast growth factor receptor (FGFR) genetic alterations, in particular a population of adult patients with locally advanced or metastatic urothelial carcinoma harboring one or more fibroblast growth factor receptor (FGFR) genetic alterations, wherein the population of patients, in particular the population of adult patients, progressed during or following at least one line of prior treatment with an anti-PD-(L)1 agent including within 12 months of neoadjuvant or adjuvant therapy, and wherein erdafitinib is administered or is to be administered in a therapeutically effective amount.
98. Erdafitinib for use in improving ORR in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, received at least one line of systemic treatment containing an anti-PD-(L)1 agent in the locally advanced or metastatic treatment, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
99. Erdafitinib for use in improving ORR in a population of patients with locally advanced or metastatic urothelial carcinoma (mUC), in particular a population of adult patients with locally advanced or metastatic urothelial carcinoma, comprising
(a) evaluating biological samples from the population of patients, in particular the population of adult patients, and wherein the population of patients, in particular the population of adult patients, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said population of patients, in particular said population of adult patients, a therapeutically effective amount of erdafitinib if one or more fibroblast growth factor receptor (FGFR) genetic alterations is present in the samples.
100. The method, use or for use according to any one of claims 25 to 39 wherein the improvement of overall survival is a median overall survival of about 12.1 months.
101. The method, use or for use according to any one of claims 25 to 39 wherein the improvement of overall survival is versus chemotherapy.
102. The method, use or for use according to any one of claims 25 to 39 wherein the improvement of overall survival is a median overall survival of about 12.1 months versus a median overall survival of about 7.8 months for chemotherapy.
103. The method, use or for use according to claim 101 or 102 wherein the chemotherapy is docetaxel or vinflunine.
104. The method, use or for use according to any one of claims 40 to 54 wherein the reduced risk of death is a reduced risk of death by about 36%.
105. The method, use or for use according to any one of claims 40 to 54 wherein the reduced risk of death is versus chemotherapy.
106. The method, use or for use according to any one of claims 40 to 54 wherein the reduced risk of death is a reduced risk of death by about 36% versus chemotherapy.
107. The method, use or for use according to claim 105 or 106 wherein the chemotherapy is docetaxel or vinflunine.
108. The method, use or for use according to any one of claims 55 to 69 wherein the improvement of PFS is a median PFS of about 5.6 months.
109. The method, use or for use according to any one of claims 55 to 69 wherein the improvement of PFS is versus chemotherapy.
110. The method, use or for use according to any one of claims 55 to 69 wherein the improvement of PFS is a median PFS of about 5.6 months versus a median PFS of about 2.7 months for chemotherapy.
111. The method, use or for use according to claim 109 or 110 wherein the chemotherapy is docetaxel or vinflunine.
112. The method, use or for use according to any one of claims 70 to 84 wherein the reduced risk of progression or death is a reduced risk of progression or death by about 42%.
113. The method, use or for use according to any one of claims 70 to 84 wherein the reduced risk of progression or death is versus chemotherapy.
114. The method, use or for use according to any one of claims 70 to 84 wherein the reduced risk of progression or death is a reduced risk of progression or death of about 42% versus chemotherapy.
115. The method, use or for use according to claim 113 or 114 wherein the chemotherapy is docetaxel or vinflunine.
116. The method, use or for use according to any one of claims 85 to 99 wherein the improvement of ORR is a ORR of about 45.6%.
117. The method, use or for use according to any one of claims 85 to 99 wherein the improvement of ORR is versus chemotherapy.
118. The method, use or for use according to any one of claims 85 to 99 wherein the improvement of ORR is a ORR of about 45.6% versus a ORR of 11.5% for chemotherapy.
119. The method, use or for use according to claim 117 or 118 wherein the chemotherapy is docetaxel or vinflunine.
120. The method, use or for use according to any one of the preceding claims wherein the one or more FGFR genetic alterations are selected from a FGFR fusion and/or mutation.
121. The method, use or for use according to any one of the preceding claims wherein the one or more FGFR genetic alterations are selected from a FGFR3 mutation and/or a FGFR2 fusion and/or a FGFR 3 fusions.
122. The method, use or for use according to any one of the preceding claims wherein the one or more FGFR genetic alterations are selected from FGFR3-TACC3, in particular FGFR3-TACC3 v1 and FGFR3-TACC3 v3; FGFR3-BAIAP2L1; FGFR2-BICC1; FGFR2-CASP7; FGFR3 R248C; FGFR3 S249C; FGFR3 G370C; and FGFR3 Y373C. I 123. The method, use or for use according to any one of claims 1 to 119 wherein the one or more FGFR genetic alterations are selected from a FGFR3 mutation and/or a FGFR3 fusion.
124. The method, use or for use according to claim 123 wherein the one or more FGFR genetic alterations are selected from FGFR3-TACC3, in particular FGFR3-TACC3 v1 and FGFR3-TACC3 v3; FGFR3-BAIAP2L1; FGFR3 R248C; FGFR3 S249C; FGFR3 G370C; and FGFR3 Y373C.
125. The method, use or for use according to any one of claims 1 to 119 wherein the one or more FGFR genetic alterations comprise or consists of FGFR3 S249C.
126. The method, use or for use according to any one of claims 1 to 119 wherein the one or more FGFR genetic alterations comprise or consists of FGFR3 Y373C.
127. The method, use or for use according to any one of claims 1 to 119 wherein the one or more FGFR genetic alterations comprise or consists of FGFR3 G370C.
128. The method, use or for use according to any one of claims 1 to 119 wherein the one or more FGFR genetic alterations comprise or consists of FGFR3 R248C.
129. The method, use or for use according to any one of claims 1 to 119 wherein the one or more FGFR genetic alterations comprise or consists of FGFR3-TACC3 v1.
130. The method, use or for use according to any one of claims 1 to 119 wherein the one or more FGFR genetic alterations comprise or consists of FGFR3-TACC3 v3.
131. The method, use or for use according to any one of claims 1 to 119 wherein the one or more FGFR genetic alterations comprise or consists of FGFR3-BAIAP2L1.
132. The method, use or for use according to any one of claims 1 to 119 wherein the one or more FGFR genetic alterations comprise or consists of FGFR2-BICC1.
133. The method, use or for use according to any one of claims 1 to 119 wherein the one or more FGFR genetic alterations comprise or consists of FGFR2-CASP7.
134. The method, use or for use according to any one of the preceding claims wherein the therapeutically effective amount of erdafitinib is 8 mg daily, in particular 8 mg once daily.
135. The method, use or for use according to any one of claims 1 to 133 wherein the therapeutically effective amount of erdafitinib is 9 mg daily, in particular 9 mg once daily.
136. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on any of day 14 to 21 after initiating erdafitinib treatment.
137. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on day 14 of erdafitinib treatment.
138. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on day 15 of erdafitinib treatment.
139. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on day 16 of erdafitinib treatment.
140. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on day 17 of erdafitinib treatment
141. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on day 18 of erdafitinib treatment.
142. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on day 19 of erdafitinib treatment.
143. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on day 20 of erdafitinib treatment.
144. The method, use or for use according to any one of claims 1 to 133 wherein the starting dose of erdafitinib is 8 mg daily, in particular once daily, with potential up-titration to 9 mg daily, in particular once daily, based on observed serum phosphate level, in particular serum phosphate level assessed on day 21 of erdafitinib treatment.
145. The method, use or for use according to any one of claims 136 to 144 wherein the erdafitinib dose is uptitrated to 9 mg daily, in particular once daily, if serum phosphate level is <5.5 mg/dL.
146. The method, use or for use according to any one of claims 136 to 144 wherein the erdafitinib dose is uptitrated to 9 mg daily, in particular once daily, if serum phosphate level is <7 mg/dL.
147. The method, use or for use according to any one of claims 136 to 144 wherein the erdafitinib dose is uptitrated to 9 mg daily, in particular once daily, if serum phosphate level is <9 mg/dL, in particular if the serum phosphate level assessed 14 to 21 days after initiating erdafitinib treatment is ranging from and including 7.00 to 8.99 mg/dL while concurrently initiating treatment with a phosphate binder such as for example sevelamer.
148. The method, use or for use according to any one of claims 136 to 147 wherein the erdafitinib dose is uptitrated to 9 mg daily, in particular once daily when there is no drug-related toxicity.
149. The method, use or for use according to any one of claims 136 to 147 wherein the erdafitinib dose is uptitrated to 9 mg daily, in particular once daily when there are no ocular disorders or Grade 2 or greater adverse reactions.
150. The method, use or for use according to any one of claims 134, 136 to 149 wherein the 8 mg dose of erdafitinib is administered or is to be administered as 2 formulations, in particular 2 tablets, each comprising 4 mg of erdafitinib.
151. The method, use or for use according to any one of claims 134, 136 to 149 wherein the 8 mg dose of erdafitinib is administered or is to be administered as 2 formulations, in particular 2 tablets, one comprising 3 mg of erdafitinib and one comprising 5 mg erdafitinib.
151. The method, use or for use according to any one of claims 135 to 149 wherein the 9 mg dose of erdafitinib is administered or is to be administered as 3 formulations, in particular 3 tablets, each comprising 3 mg of erdafitinib.
152. The method, use or for use according to any one of claims 135 to 149 wherein the 9 mg dose of erdafitinib is administered or is to be administered as 2 formulations, in particular 2 tablets, one comprising 4 mg of erdafitinib and one comprising 5 mg erdafitinib.
1. A method of treating locally advanced or metastatic urothelial carcinoma (mUC) harboring one or more fibroblast growth factor receptor (FGFR) genetic alterations, comprising administering to a subject in need thereof, in particular a patient with locally advanced or metastatic urothelial carcinoma, more in particular an adult patient with locally advanced or metastatic urothelial carcinoma, a therapeutically effective amount of erdafitinib, wherein the subject, in particular the patient, more in particular the adult patient, received at least one line of systemic treatment containing an anti-PD-(L)1 agent in the locally advanced or metastatic treatment.
2. A method of treating locally advanced or metastatic urothelial carcinoma (mUC), harboring one or more fibroblast growth factor receptor (FGFR) genetic alterations, comprising administering to a subject in need thereof, in particular a patient with locally advanced or metastatic urothelial carcinoma, more in particular an adult patient with locally advanced or metastatic urothelial carcinoma, a therapeutically effective amount of erdafitinib, wherein the subject, in particular the patient, more in particular the adult patient, progressed on or after ≥1 prior treatment that included an anti-PD-(L)1 agent.
3. A method of treating locally advanced or metastatic urothelial carcinoma (mUC) harboring one or more FGFR genetic alterations, comprising administering to a subject in need thereof, in particular a patient with locally advanced or metastatic urothelial carcinoma, more in particular an adult patient with locally advanced or metastatic urothelial carcinoma, a therapeutically effective amount of erdafitinib, wherein the subject, in particular the patient, more in particular the adult patient, progressed during or following at least one line of prior treatment with an anti-PD-(L)1 agent including within 12 months of neoadjuvant or adjuvant therapy.
4. A method for the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who received at least one line of systemic treatment containing an anti-PD-(L)1 agent in the locally advanced or metastatic treatment setting, comprising administering to the subject, in particular the patient, more in particular the adult patient, a therapeutically effective amount of erdafitinib.
5. A method for the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who progressed on or after ≥1 prior treatment that included an anti-PD-(L)1 agent, comprising administering to the subject, in particular the patient, more in particular the adult patient, a therapeutically effective amount of erdafitinib.
6. A method for the treatment of a subject in need thereof, in particular a patient with locally advanced or metastatic urothelial carcinoma, more in particular an adult patient with locally advanced or metastatic urothelial carcinoma, that has
   one or more FGFR genetic alterations and
   progressed during or following at least one line of prior treatment with an anti-PD-(L)1 agent including within 12 months of neoadjuvant or adjuvant therapy, comprising administering to the subject, in particular the patient, more in particular the adult patient, a therapeutically effective amount of erdafitinib.
7. A method of treating locally advanced or metastatic urothelial carcinoma (mUC) comprising
(a) evaluating a biological sample from a subject in need of the treatment, in particular a patient with locally advanced or metastatic urothelial carcinoma, more in particular an adult patient with locally advanced or metastatic urothelial carcinoma, and wherein the subject, in particular the patient, more in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample.
8. A method for the treatment of a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the subject, in particular the patient, more in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to the subject, in particular the patient, more in particular the adult patient, a therapeutically effective amount of erdafitinib if one or more fibroblast growth factor receptor (FGFR) genetic alterations is present in the sample.
9. A method of improving overall survival in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib.
10. A method of improving overall survival in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient, a therapeutically effective amount of erdafitinib.
11. A method of improving overall survival in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient, a therapeutically effective amount of erdafitinib.
12. A method of improving overall survival in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
13. A method of improving overall survival in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
14. A method of reducing risk of death in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib.
15. A method of reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient, a therapeutically effective amount of erdafitinib.
16. A method of reducing risk of death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient, a therapeutically effective amount of erdafitinib.
17. A method of reducing risk of death in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
18. A method of reducing risk of death in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
19. A method of improving median PFS in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib.
20. A method of improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient, a therapeutically effective amount of erdafitinib.
21. A method of improving median PFS in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient, a therapeutically effective amount of erdafitinib.
22. A method of improving median PFS in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
23. A method of improving median PFS in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
24. A method of reducing risk of progression or death in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib.
25. A method of reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient a therapeutically effective amount of erdafitinib.
26. A method of reducing risk of progression or death in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient a therapeutically effective amount of erdafitinib.
27. A method of reducing risk of progression or death in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
28. A method of reducing risk of progression or death in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
29. A method of improving ORR in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib.
30. A method of improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed on or after ≥1 prior treatment that included an anti-PD-(L)1 agent, comprising administering to said patient, in particular said adult patient a therapeutically effective amount of erdafitinib.
31. A method of improving ORR in a patient with locally advanced or metastatic urothelial carcinoma (mUC) harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient a therapeutically effective amount of erdafitinib.
32. A method of improving ORR in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
33. A method of improving ORR in a patient with locally advanced or metastatic urothelial carcinoma (mUC), in particular an adult patient with locally advanced or metastatic urothelial carcinoma, comprising
(a) evaluating a biological sample from the patient, in particular the adult patient, wherein the patient, in particular the adult patient, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib if one or more fibroblast growth factor receptor (FGFR) genetic alterations is present in the sample.
34. The method according to any one of claims 9 to 13 wherein the improvement of overall survival is a median overall survival of about 12.1 months.
35. The method according to any one of claims 9 to 13 wherein the improvement of overall survival is versus chemotherapy.
36. The method according to any one of claims 9 to 13 wherein the improvement of overall survival is a median overall survival of about 12.1 months versus a median overall survival of about 7.8 months for chemotherapy.
37. The method according to claim 35 or 36 wherein the chemotherapy is docetaxel or vinflunine.
38. The method according to any one of claims 14 to 18 wherein the reduced risk of death is a reduced risk of death by about 36%.
39. The method according to any one of claims 14 to 18 wherein the reduced risk of death is versus chemotherapy.
40. The method according to any one of claims 14 to 18 wherein the reduced risk of death is a reduced risk of death by about 36% versus chemotherapy.
41. The method according to claim 39 or 40 wherein the chemotherapy is docetaxel or vinflunine.
42. The method according to any one of claims 19 to 23 wherein the improvement of PFS is a median PFS of about 5.6 months.
43. The method according to any one of claims 19 to 23 wherein the improvement of PFS is versus chemotherapy.
44. The method according to any one of claims 19 to 23 wherein the improvement of PFS is a median PFS of about 5.6 months versus a median PFS of about 2.7 months for chemotherapy.
45. The method according to claim 43 or 44 wherein the chemotherapy is docetaxel or vinflunine.
46. The method according to any one of claims 24 to 28 wherein the reduced risk of progression or death is a reduced risk of progression or death by about 42%.
47. The method according to any one of claims 24 to 28 wherein the reduced risk of progression or death is versus chemotherapy.
48. The method according to any one of claims 24 to 28 wherein the reduced risk of progression or death is a reduced risk of progression or death of about 42% versus chemotherapy.
49. The method according to claim 47 to 48 wherein the chemotherapy is docetaxel or vinflunine.
50. The method according to any one of claims 29 to 33 wherein the improvement of ORR is a ORR of about 45.6%.
51. The method according to any one of claims 29 to 33 wherein the improvement of ORR is versus chemotherapy.
52. The method according to any one of claims 29 to 33 wherein the improvement of ORR is a ORR of about 45.6% versus a ORR of 11.5% for chemotherapy.
53. The method according to claim 51 or 52 wherein the chemotherapy is docetaxel or vinflunine.
54. The method according to any one of the preceding claims wherein the one or more FGFR genetic alterations are selected from a FGFR fusion and/or mutation.
55. The method according to any one of the preceding claims wherein the one or more FGFR genetic alterations are selected from a FGFR3 mutation and/or a FGFR2 fusion and/or a FGFR 3 fusions.
56. The method according to any one of the preceding claims wherein the one or more FGFR genetic alterations are selected from FGFR3-TACC3, in particular FGFR3-TACC3 v1 and FGFR3-TACC3 v3; FGFR3-BAIAP2L1; FGFR2:BICC1; FGFR2:CASP7; FGFR3 R248C; FGFR3 S249C; FGFR3 G370C; and FGFR3 Y373C.
57. The method according to any one of claims 1 to 53 wherein the one or more FGFR genetic alterations are selected from a FGFR3 mutation and/or a FGFR3 fusion.
58. The method according to claim 57 wherein the one or more FGFR genetic alterations are selected from FGFR3-TACC3, in particular FGFR3-TACC3 v1 and FGFR3-TACC3 v3; FGFR3-BAIAP2L1; FGFR3 R248C; FGFR3 S249C; FGFR3 G370C; and FGFR3 Y373C.
59. The method according to any one of claims 1 to 53 wherein the one or more FGFR genetic alterations comprise or consists of FGFR3 S249C.
60. The method according to any one of claims 1 to 53 wherein the one or more FGFR genetic alterations comprise or consists of FGFR3 Y373C.
61. The method according to any one of claims 1 to 53 wherein the one or more FGFR genetic alterations comprise or consists of FGFR3 G370C.
62. The method according to any one of claims 1 to 53 wherein the one or more FGFR genetic alterations comprise or consists of FGFR3 R248C.
63. The method according to any one of claims 1 to 53 wherein the one or more FGFR genetic alterations comprise or consists of FGFR3-TACC3 v1.
64. The method according to any one of claims 1 to 53 wherein the one or more FGFR genetic alterations comprise or consists of FGFR3-TACC3 v3.
65. The method according to any one of claims 1 to 53 wherein the one or more FGFR genetic alterations comprise or consists of FGFR3-BAIAP2L1.
66. The method according to any one of claims 1 to 53 wherein the one or more FGFR genetic alterations comprise or consists of FGFR2:BICC1.
67. The method according to any one of claims 1 to 53 wherein the one or more FGFR genetic alterations comprise or consists of FGFR2:CASP7.
68. The method according to any one of the preceding claims wherein the therapeutically effective amount of erdafitinib is 8 mg daily, in particular 8 mg once daily.
69. The method according to any one of claims 1 to 67 wherein the therapeutically effective amount of erdafitinib is 9 mg daily, in particular 9 mg once daily.
70. The method according to any one of claims 1 to 67 wherein the starting dose of erdafitinib is 8 mg daily, in particular once daily, with potential up-titration to 9 mg daily, in particular once daily, based on observed serum phosphate level, in particular serum phosphate level assessed on any of day 14 to 21 after initiating erdafitinib treatment.
71. The method according to any one of claims 1 to 67 wherein the starting dose of erdafitinib is 8 mg daily, in particular once daily, with potential up-titration to 9 mg daily, in particular once daily, based on observed serum phosphate level, in particular serum phosphate level assessed on day 14 of erdafitinib treatment.
72. The method according to any one of claims 1 to 67 wherein the starting dose of erdafitinib is 8 mg daily, in particular once daily, with potential up-titration to 9 mg daily, in particular once daily, based on observed serum phosphate level, in particular serum phosphate level assessed on day 15 of erdafitinib treatment.
73. The method according to any one of claims 1 to 67 wherein the starting dose of erdafitinib is 8 mg daily, in particular once daily, with potential up-titration to 9 mg daily, in particular once daily, based on observed serum phosphate level, in particular serum phosphate level assessed on day 16 of erdafitinib treatment.
74. The method according to any one of claims 1 to 67 wherein the starting dose of erdafitinib is 8 mg daily, in particular once daily, with potential up-titration to 9 mg daily, in particular once daily, based on observed serum phosphate level, in particular serum phosphate level assessed on day 17 of erdafitinib treatment
75. The method according to any one of claims 1 to 67 wherein the starting dose of erdafitinib is 8 mg daily, in particular once daily, with potential up-titration to 9 mg daily, in particular once daily, based on observed serum phosphate level, in particular serum phosphate level assessed on day 18 of erdafitinib treatment.
76. The method according to any one of claims 1 to 67 wherein the starting dose of erdafitinib is 8 mg daily, in particular once daily, with potential up-titration to 9 mg daily, in particular once daily, based on observed serum phosphate level, in particular serum phosphate level assessed on day 19 of erdafitinib treatment.
77. The method according to any one of claims 1 to 67 wherein the starting dose of erdafitinib is 8 mg daily, in particular once daily, with potential up-titration to 9 mg daily, in particular once daily, based on observed serum phosphate level, in particular serum phosphate level assessed on day 20 of erdafitinib treatment.
78. The method according to any one of claims 1 to 67 wherein the starting dose of erdafitinib is 8 mg daily, in particular once daily, with potential up-titration to 9 mg daily, in particular once daily, based on observed serum phosphate level, in particular serum phosphate level assessed on day 21 of erdafitinib treatment.
79. The method according to any one of claims 70 to 78 wherein the erdafitinib dose is uptitrated to 9 mg daily, in particular once daily, if serum phosphate level is <5.5 mg/dL.
80. The method according to any one of claims 70 to 78 wherein the erdafitinib dose is uptitrated to 9 mg daily, in particular once daily, if serum phosphate level is <7 mg/dL.
81. The method according to any one of claims 70 to 78 wherein the erdafitinib dose is uptitrated to 9 mg daily, in particular once daily, if serum phosphate level is <9 mg/dL, in particular if the serum phosphate level assessed 14 to 21 days after initiating erdafitinib treatment is ranging from and including 7.00 to 8.99 mg/dL while concurrently initiating treatment with a phosphate binder such as for example sevelamer.
82. The method according to any one of claims 70 to 81 wherein the erdafitinib dose is uptitrated to 9 mg daily, in particular once daily when there is no drug-related toxicity.
83. The method according to any one of claims 70 to 81 wherein the erdafitinib dose is uptitrated to 9 mg daily, in particular once daily when there are no ocular disorders or Grade 2 or greater adverse reactions.
84. The method according to any one of claims 68, 70 to 83 wherein the 8 mg dose of erdafitinib is administered or is to be administered as 2 formulations, in particular 2 tablets, each comprising 4 mg of erdafitinib.
85. The method according to any one of claims 68, 70 to 83 wherein the 8 mg dose of erdafitinib is administered or is to be administered as 2 formulations, in particular 2 tablets, one comprising 3 mg of erdafitinib and one comprising 5 mg erdafitinib.
86. The method according to any one of claims 69 to 83 wherein the 9 mg dose of erdafitinib is administered or is to be administered as 3 formulations, in particular 3 tablets, each comprising 3 mg of erdafitinib.
87. The method according to any one of claims 69 to 83 wherein the 9 mg dose of erdafitinib is administered or is to be administered as 2 formulations, in particular 2 tablets, one comprising 4 mg of erdafitinib and one comprising 5 mg erdafitinib.

1. A method of treating locally advanced or metastatic urothelial carcinoma harboring one or more fibroblast growth factor receptor (FGFR) genetic alterations, comprising administering to a subject in need thereof, in particular a patient with locally advanced or metastatic urothelial carcinoma, more in particular an adult patient with locally advanced or metastatic urothelial carcinoma, a therapeutically effective amount of erdafitinib or a pharmaceutically acceptable salt thereof, wherein the subject, in particular the patient, more in particular the adult patient, received at least one line of systemic treatment containing an anti-PD-(L)1 agent in the locally advanced or metastatic treatment.

2. A method of treating locally advanced or metastatic urothelial carcinoma, harboring one or more fibroblast growth factor receptor (FGFR) genetic alterations, comprising administering to a subject in need thereof, in particular a patient with locally advanced or metastatic urothelial carcinoma, more in particular an adult patient with locally advanced or metastatic urothelial carcinoma, a therapeutically effective amount of erdafitinib or a pharmaceutically acceptable salt thereof, wherein the urothelial carcinoma, in particular the locally advanced or metastatic urothelial carcinoma, progressed on or after ≥1 prior treatment that included an anti-PD-(L)1 agent.

3. A method of treating locally advanced or metastatic urothelial carcinoma harboring one or more FGFR genetic alterations, comprising administering to a subject in need thereof, in particular a patient with locally advanced or metastatic urothelial carcinoma, more in particular an adult patient with locally advanced or metastatic urothelial carcinoma, a therapeutically effective amount of erdafitinib or a pharmaceutically acceptable salt thereof, wherein the urothelial carcinoma, in particular the locally advanced or metastatic urothelial carcinoma, progressed during or following at least one line of prior treatment with an anti-PD-(L)1 agent including within 12 months of neoadjuvant or adjuvant therapy.

4. A method for the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and who received at least one line of systemic treatment containing an anti-PD-(L)1 agent in the locally advanced or metastatic treatment setting, comprising administering to the subject, in particular the patient, more in particular the adult patient, a therapeutically effective amount of erdafitinib or a pharmaceutically acceptable salt thereof.

5. A method for the treatment of a subject in need thereof, in particular a patient with locally advanced or metastatic urothelial carcinoma, more in particular an adult patient with locally advanced or metastatic urothelial carcinoma, whose tumor has one or more fibroblast growth factor receptor (FGFR) genetic alterations, and whose urothelial carcinoma, in particular locally advanced or metastatic urothelial carcinoma, progressed on or after ≥1 prior treatment that included an anti-PD-(L)1 agent, comprising administering to the subject, in particular the patient, more in particular the adult patient, a therapeutically effective amount of erdafitinib or a pharmaceutically acceptable salt thereof.

6. A method for the treatment of a subject in need thereof, in particular a patient with locally advanced or metastatic urothelial carcinoma, more in particular an adult patient with locally advanced or metastatic urothelial carcinoma,
that has one or more FGFR genetic alterations and
whose urothelial carcinoma, in particular locally advanced or metastatic urothelial carcinoma, progressed during or following at least one line of prior treatment with an anti-PD-(L)1 agent including within 12 months of neoadjuvant or adjuvant therapy, comprising administering to the subject, in particular the patient, more in particular the adult patient, a therapeutically effective amount of erdafitinib or a pharmaceutically acceptable salt thereof.

7. A method of treating locally advanced or metastatic urothelial carcinoma comprising
(a) evaluating a biological sample from a subject in need of the treatment, in particular a patient with locally advanced or metastatic urothelial carcinoma, more in particular an adult patient with locally advanced or metastatic urothelial carcinoma, and wherein the subject, in particular the patient, more in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to the subject, in particular the patient, more in particular the adult patient, a therapeutically effective amount of erdafitinib or a pharmaceutically acceptable salt thereof if one or more fibroblast growth factor receptor (FGFR) genetic alterations is present in the sample.

8. A method for the treatment of a subject in need thereof, in particular a patient with locally advanced or metastatic urothelial carcinoma, more in particular an adult patient with locally advanced or metastatic urothelial carcinoma, comprising
(a) evaluating a biological sample from the subject, in particular the patient, more in particular the adult patient, and wherein the urothelial carcinoma, in particular the locally advanced or metastatic urothelial carcinoma, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to the subject, in particular the patient, more in particular the adult patient, a therapeutically effective amount of erdafitinib or a pharmaceutically acceptable salt thereof if one or more fibroblast growth factor receptor (FGFR) genetic alterations is present in the sample.

9. A method of improving overall survival in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib or a pharmaceutically acceptable salt thereof.

10. A method of improving overall survival in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the urothelial carcinoma, in particular the locally advanced or metastatic urothelial carcinoma, progressed on or after ≥1 prior treatment that included an anti-PD-(L)1 agent, comprising administering to said patient, in particular said adult patient, a therapeutically effective amount of erdafitinib or a pharmaceutically acceptable salt thereof.

11. A method of improving overall survival in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the urothelial carcinoma, in particular the locally advanced or metastatic urothelial carcinoma, progressed during or following at least one line of prior treatment with an anti-PD-(L)1 agent including within 12 months of neoadjuvant or adjuvant therapy, comprising administering to said patient, in particular said adult patient, a therapeutically effective amount of erdafitinib or a pharmaceutically acceptable salt thereof.

12. A method of improving overall survival in a patient with locally advanced or metastatic urothelial carcinoma, in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib or a pharmaceutically acceptable salt thereof if one or more fibroblast growth factor receptor (FGFR) genetic alterations is present in the sample.

13. A method of improving overall survival in a patient with locally advanced or metastatic urothelial carcinoma, in particular an adult patient with locally advanced or metastatic urothelial carcinoma, comprising
(a) evaluating a biological sample from the patient, in particular the adult patient, wherein the urothelial carcinoma, in particular the locally advanced or metastatic urothelial carcinoma, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib or a pharmaceutically acceptable salt thereof if one or more fibroblast growth factor receptor (FGFR) genetic alterations is present in the sample.

14. A method of reducing risk of death in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib or a pharmaceutically acceptable salt thereof.

15. A method of reducing risk of death in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the urothelial carcinoma, in particular the locally advanced or metastatic urothelial carcinoma, progressed on or after ≥1 prior treatment that included an anti-PD-(L)1 agent, comprising administering to said patient, in particular said adult patient, a therapeutically effective amount of erdafitinib or a pharmaceutically acceptable salt thereof.

16. A method of reducing risk of death in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the urothelial carcinoma, in particular the locally advanced or metastatic urothelial carcinoma, progressed during or following at least one line of prior treatment with an anti-PD-(L)1 agent including within 12 months of neoadjuvant or adjuvant therapy, comprising administering to said patient, in particular said adult patient, a therapeutically effective amount of erdafitinib or a pharmaceutically acceptable salt thereof.

17. A method of reducing risk of death in a patient with locally advanced or metastatic urothelial carcinoma, in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib or a pharmaceutically acceptable salt thereof if one or more fibroblast growth factor receptor (FGFR) genetic alterations is present in the sample.

18. A method of reducing risk of death in a patient with locally advanced or metastatic urothelial carcinoma, in particular an adult patient with locally advanced or metastatic urothelial carcinoma, comprising
(a) evaluating a biological sample from the patient, in particular the adult patient, wherein the urothelial carcinoma, in particular the locally advanced or metastatic urothelial carcinoma, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib or a pharmaceutically acceptable salt thereof if one or more fibroblast growth factor receptor (FGFR) genetic alterations is present in the sample.

19. A method of improving median PFS in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib or a pharmaceutically acceptable salt thereof.

20. A method of improving median PFS in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the urothelial carcinoma, in particular the locally advanced or metastatic urothelial carcinoma, progressed on or after ≥1 prior treatment that included an anti-PD-(L)1 agent, comprising administering to said patient, in particular said adult patient, a therapeutically effective amount of erdafitinib or a pharmaceutically acceptable salt thereof.

21. A method of improving median PFS in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the urothelial carcinoma, in particular the locally advanced or metastatic urothelial carcinoma, progressed during or following at least one line of prior treatment with an anti-PD-(L)1 agent including within 12 months of neoadjuvant or adjuvant therapy, comprising administering to said patient, in particular said adult patient, a therapeutically effective amount of erdafitinib or a pharmaceutically acceptable salt thereof.

22. A method of improving median PFS in a patient with locally advanced or metastatic urothelial carcinoma, in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib or a pharmaceutically acceptable salt thereof if one or more fibroblast growth factor receptor (FGFR) genetic alterations is present in the sample.

23. A method of improving median PFS in a patient with locally advanced or metastatic urothelial carcinoma, in particular an adult patient with locally advanced or metastatic urothelial carcinoma, comprising
(a) evaluating a biological sample from the patient, in particular the adult patient, wherein the urothelial carcinoma, in particular the locally advanced or metastatic urothelial carcinoma, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib or a pharmaceutically acceptable salt thereof if one or more fibroblast growth factor receptor (FGFR) genetic alterations is present in the sample.

24. A method of reducing risk of progression or death in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib or a pharmaceutically acceptable salt thereof.

25. A method of reducing risk of progression or death in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the urothelial carcinoma, in particular the locally advanced or metastatic urothelial carcinoma, progressed on or after ≥1 prior treatment that included an anti-PD-(L)1 agent, comprising administering to said patient, in particular said adult patient, a therapeutically effective amount of erdafitinib or a pharmaceutically acceptable salt thereof.

26. A method of reducing risk of progression or death in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the urothelial carcinoma, in particular the locally advanced or metastatic urothelial carcinoma, progressed during or following at least one line of prior treatment with an anti-PD-(L)1 agent including within 12 months of neoadjuvant or adjuvant therapy, comprising administering to said patient, in particular said adult patient, a therapeutically effective amount of erdafitinib or a pharmaceutically acceptable salt thereof.

27. A method of reducing risk of progression or death in a patient with locally advanced or metastatic urothelial carcinoma, in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib or a pharmaceutically acceptable salt thereof if one or more fibroblast growth factor receptor (FGFR) genetic alterations is present in the sample.

28. A method of reducing risk of progression or death in a patient with locally advanced or metastatic urothelial carcinoma, in particular an adult patient with locally advanced or metastatic urothelial carcinoma, comprising
(a) evaluating a biological sample from the patient, in particular the adult patient, wherein the urothelial carcinoma, in particular the locally advanced or metastatic urothelial carcinoma, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib or a pharmaceutically acceptable salt thereof if one or more fibroblast growth factor receptor (FGFR) genetic alterations is present in the sample.

29. A method of improving ORR in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, received at least one line of systemic treatment containing an anti-PD-(L)1 agent in the locally advanced or metastatic treatment, comprising administering to said patient, in particular said adult patient, a therapeutically effective amount of erdafitinib or a pharmaceutically acceptable salt thereof.

30. A method of improving ORR in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the urothelial carcinoma, in particular the locally advanced or metastatic urothelial carcinoma, progressed on or after ≥1 prior treatment that included an anti-PD-(L)1 agent, comprising administering to said patient, in particular said adult patient, a therapeutically effective amount of erdafitinib or a pharmaceutically acceptable salt thereof.

31. A method of improving ORR in a patient with locally advanced or metastatic urothelial carcinoma harboring one or more fibroblast growth factor receptor (FGFR) genetic alterations, in particular an adult patient with locally advanced or metastatic urothelial carcinoma harboring one or more fibroblast growth factor receptor (FGFR) genetic alterations, wherein the patient, in particular the adult patient, progressed during or following at least one line of prior treatment with an anti-PD-(L)1 agent including within 12 months of neoadjuvant or adjuvant therapy, comprising administering to said patient, in particular said adult patient, a therapeutically effective amount of erdafitinib or a pharmaceutically acceptable salt thereof.

32. A method of improving ORR in a patient with locally advanced or metastatic urothelial carcinoma, in particular an adult patient with locally advanced or metastatic urothelial carcinoma, comprising
(a) evaluating a biological sample from the patient, in particular the adult patient, wherein the patient, in particular the adult patient, received at least one line of systemic treatment containing an anti-PD-(L)1 agent in the locally advanced or metastatic treatment, for the presence of one or more fibroblast growth factor receptor (FGFR) genetic alterations; and
(b) administering to said patient, in particular said adult patient, a therapeutically effective amount of erdafitinib or a pharmaceutically acceptable salt thereof if one or more fibroblast growth factor receptor (FGFR) genetic alterations is present in the sample.

33. A method of improving ORR in a patient with locally advanced or metastatic urothelial carcinoma, in particular an adult patient with locally advanced or metastatic urothelial carcinoma, comprising
(a) evaluating a biological sample from the patient, in particular the adult patient, wherein the urothelial carcinoma, in particular the locally advanced or metastatic urothelial carcinoma, progressed during or following at least one line of prior treatment with an anti-PD-(L)1 agent including within 12 months of neoadjuvant or adjuvant therapy, for the presence of one or more fibroblast growth factor receptor (FGFR) genetic alterations; and
(b) administering to said patient, in particular said adult patient, a therapeutically effective amount of erdafitinib or a pharmaceutically acceptable salt thereof if one or more fibroblast growth factor receptor (FGFR) genetic alterations is present in the sample.

34. The method according to any one of claims 9 to 13 wherein the improvement of overall survival is a median overall survival of about 12.1 months.

35. The method according to any one of claims 9 to 13 wherein the improvement of overall survival is versus chemotherapy.

36. The method according to any one of claims 9 to 13 wherein the improvement of overall survival is a median overall survival of about 12.1 months versus a median overall survival of about 7.8 months for chemotherapy.

37. The method according to claim 35 or 36 wherein the chemotherapy is docetaxel or vinflunine.

38. The method according to any one of claims 14 to 18 wherein the reduced risk of death is a reduced risk of death by about 36%.

39. The method according to any one of claims 14 to 18 wherein the reduced risk of death is versus chemotherapy.

40. The method according to any one of claims 14 to 18 wherein the reduced risk of death is a reduced risk of death by about 36% versus chemotherapy.

41. The method according to claim 39 or 40 wherein the chemotherapy is docetaxel or vinflunine.

42. The method according to any one of claims 19 to 23 wherein the improvement of PFS is a median PFS of about 5.6 months.

43. The method according to any one of claims 19 to 23 wherein the improvement of PFS is versus chemotherapy.

44. The method according to any one of claims 19 to 23 wherein the improvement of PFS is a median PFS of about 5.6 months versus a median PFS of about 2.7 months for chemotherapy.

45. The method according to claim 43 or 44 wherein the chemotherapy is docetaxel or vinflunine.

46. The method according to any one of claims 24 to 28 wherein the reduced risk of progression or death is a reduced risk of progression or death by about 42%.

47. The method according to any one of claims 24 to 28 wherein the reduced risk of progression or death is versus chemotherapy.

48. The method according to any one of claims 24 to 28 wherein the reduced risk of progression or death is a reduced risk of progression or death of about 42% versus chemotherapy.

49. The method according to claim 47 to 48 wherein the chemotherapy is docetaxel or vinflunine.

50. The method according to any one of claims 29 to 33 wherein the improvement of ORR is a confirmed ORR of about 35.3%.

51. The method according to any one of claims 29 to 33 wherein the improvement of ORR is versus chemotherapy.

52. The method according to any one of claims 29 to 33 wherein the improvement of ORR is a confirmed ORR of about 35.3% versus a confirmed ORR of 8.5% for chemotherapy.

53. The method according to claim 51 or 52 wherein the chemotherapy is docetaxel or vinflunine.

54. The method according to any one of the preceding claims wherein the one or more FGFR genetic alterations are selected from a FGFR fusion and/or mutation.

55. The method according to any one of the preceding claims wherein the one or more FGFR genetic alterations are selected from a FGFR3 mutation and/or a FGFR2 fusion and/or a FGFR3 fusion.

56. The method according to any one of the preceding claims wherein the one or more FGFR genetic alterations are selected from FGFR3-TACC3, in particular FGFR3-TACC3 v1 and FGFR3-TACC3 v3; FGFR3-BAIAP2L1; FGFR2:BICC1; FGFR2:CASP7; FGFR3 R248C; FGFR3 S249C; FGFR3 G370C; and FGFR3 Y373C. I

57. The method according to any one of claims 1 to 53 wherein the one or more FGFR genetic alterations are selected from a FGFR3 mutation and/or a FGFR3 fusion.

58. The method according to claim 57 wherein the one or more FGFR genetic alterations are selected from FGFR3-TACC3, in particular FGFR3-TACC3 v1 and FGFR3-TACC3 v3; FGFR3-BAIAP2L1; FGFR3 R248C; FGFR3 S249C; FGFR3 G370C; and FGFR3 Y373C.

59. The method according to any one of claims 1 to 53 wherein the one or more FGFR genetic alterations comprise or consists of FGFR3 S249C.

60. The method according to any one of claims 1 to 53 wherein the one or more FGFR genetic alterations comprise or consists of FGFR3 Y373C.

61. The method according to any one of claims 1 to 53 wherein the one or more FGFR genetic alterations comprise or consists of FGFR3 G370C.

62. The method according to any one of claims 1 to 53 wherein the one or more FGFR genetic alterations comprise or consists of FGFR3 R248C.

63. The method according to any one of claims 1 to 53 wherein the one or more FGFR genetic alterations comprise or consists of FGFR3-TACC3 v1.

64. The method according to any one of claims 1 to 53 wherein the one or more FGFR genetic alterations comprise or consists of FGFR3-TACC3 v3.

65. The method according to any one of claims 1 to 53 wherein the one or more FGFR genetic alterations comprise or consists of FGFR3-BAIAP2L1.

66. The method according to any one of claims 1 to 53 wherein the one or more FGFR genetic alterations comprise or consists of FGFR2:BICC1.

67. The method according to any one of claims 1 to 53 wherein the one or more FGFR genetic alterations comprise or consists of FGFR2:CASP7.

68. The method according to any one of the preceding claims wherein the therapeutically effective amount of erdafitinib or a pharmaceutically acceptable salt thereof is 8 mg base equivalent daily, in particular 8 mg base equivalent once daily.

69. The method according to any one of claims 1 to 67 wherein the therapeutically effective amount of erdafitinib or a pharmaceutically acceptable salt thereof is 9 mg base equivalent daily, in particular 9 mg base equivalent once daily.

70. The method according to any one of claims 1 to 67 wherein the starting dose of erdafitinib or a pharmaceutically acceptable salt thereof is 8 mg base equivalent daily, in particular once daily, with potential up-titration to 9 mg base equivalent daily, in particular once daily, based on observed serum phosphate level, in particular serum phosphate level assessed on any of day 14 to 21 after initiating erdafitinib treatment.

71. The method according to any one of claims 1 to 67 wherein the starting dose of erdafitinib or a pharmaceutically acceptable salt thereof is 8 mg base equivalent daily, in particular once daily, with potential up-titration to 9 mg base equivalent daily, in particular once daily, based on observed serum phosphate level assessed on day 14 of erdafitinib treatment.

72. The method according to any one of claims 1 to 67 wherein the starting dose of erdafitinib or a pharmaceutically acceptable salt thereof is 8 mg base equivalent daily, in particular once daily, with potential up-titration to 9 mg base equivalent daily, in particular once daily, based on observed serum phosphate level assessed on day 15 of erdafitinib treatment.

73. The method according to any one of claims 1 to 67 wherein the starting dose of erdafitinib or a pharmaceutically acceptable salt thereof is 8 mg base equivalent daily, in particular once daily, with potential up-titration to 9 mg base equivalent daily, in particular once daily, based on observed serum phosphate level assessed on day 16 of erdafitinib treatment.

74. The method according to any one of claims 1 to 67 wherein the starting dose of erdafitinib or a pharmaceutically acceptable salt thereof is 8 mg base equivalent daily, in particular once daily, with potential up-titration to 9 mg base equivalent daily, in particular once daily, based on observed serum phosphate level assessed on day 17 of erdafitinib treatment

75. The method according to any one of claims 1 to 67 wherein the starting dose of erdafitinib or a pharmaceutically acceptable salt thereof is 8 mg base equivalent daily, in particular once daily, with potential up-titration to 9 mg base equivalent daily, in particular once daily, based on observed serum phosphate level assessed on day 18 of erdafitinib treatment.

76. The method according to any one of claims 1 to 67 wherein the starting dose of erdafitinib or a pharmaceutically acceptable salt thereof is 8 mg base equivalent daily, in particular once daily, with potential up-titration to 9 mg base equivalent daily, in particular once daily, based on observed serum phosphate level assessed on day 19 of erdafitinib treatment.

77. The method according to any one of claims 1 to 67 wherein the starting dose of erdafitinib or a pharmaceutically acceptable salt thereof is 8 mg base equivalent daily, in particular once daily, with potential up-titration to 9 mg base equivalent daily, in particular once daily, based on observed serum phosphate level assessed on day 20 of erdafitinib treatment.

78. The method according to any one of claims 1 to 67 wherein the starting dose of erdafitinib or a pharmaceutically acceptable salt thereof is 8 mg base equivalent daily, in particular once daily, with potential up-titration to 9 mg base equivalent daily, in particular once daily, based on observed serum phosphate level assessed on day 21 of erdafitinib treatment.

79. The method according to any one of claims 70 to 78 wherein the erdafitinib dose is uptitrated to 9 mg base equivalent daily, in particular once daily, if serum phosphate level is <5.5 mg/dL.

80. The method according to any one of claims 70 to 78 wherein the erdafitinib dose is uptitrated to 9 mg base equivalent daily, in particular once daily, if serum phosphate level is <7 mg/dL.

81. The method according to any one of claims 70 to 78 wherein the erdafitinib dose is uptitrated to 9 mg base equivalent daily, in particular once daily, if serum phosphate level is <9 mg/dL.

82. The method according to any one of claims 70 to 78 wherein the erdafitinib dose is uptitrated to 9 mg base equivalent daily, in particular once daily, if serum phosphate level is <9 mg/dL, in particular if the serum phosphate level assessed 14 to 21 days after initiating erdafitinib treatment is ranging from and including 7.00 to 8.99 mg/dL while concurrently initiating treatment with a phosphate binder such as for example sevelamer.

83. The method according to any one of claims 70 to 82 wherein the erdafitinib dose is uptitrated to 9 mg base equivalent daily, in particular once daily, when there is no drug-related toxicity.

84. The method according to any one of claims 70 to 82 wherein the erdafitinib dose is uptitrated to 9 mg base equivalent daily, in particular once daily, when there are no ocular disorders or Grade 2 or greater adverse reactions.

85. The method according to any one of claims 68, 70 to 84 wherein the erdafitinib dose of 8 mg base equivalent is administered or is to be administered as 2 formulations, in particular 2 tablets, each comprising 4 mg of erdafitinib base equivalent.

86. The method according to any one of claims 68, 70 to 84 wherein the erdafitinib dose of 8 mg base equivalent is administered or is to be administered as 2 formulations, in particular 2 tablets, one comprising 3 mg of erdafitinib base equivalent and one comprising 5 mg erdafitinib base equivalent.

87. The method according to any one of claims 69 to 84 wherein the erdafitinib dose of 9 mg base equivalent is administered or is to be administered as 3 formulations, in particular 3 tablets, each comprising 3 mg of erdafitinib base equivalent.

88. The method according to any one of claims 69 to 84 wherein the erdafitinib dose of 9 mg base equivalent is administered or is to be administered as 2 formulations, in particular 2 tablets, one comprising 4 mg of erdafitinib base equivalent and one comprising 5 mg erdafitinib base equivalent.

89. The method according to any one of claims 1 to 88 wherein erdafitinib or a pharmaceutically acceptable salt thereof is erdafitinib.


SEQUENCE LISTING (10 sequences)

SEQ ID NO: 1 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - DNA, 2850 bp, Homo sapiens
  source 1..2850: mol_type = other DNA
  variation 1..2274: note = FGFR3
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 3 - DNA, 2955 bp, Homo sapiens
  source 1..2955: mol_type = other DNA
  variation 1..2274: note = FGFR3
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 4 - DNA, 3765 bp, Homo sapiens
  source 1..3765: mol_type = other DNA
  variation 1..2280: note = FGFR3
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagtcctggatcagtgagagtgtggaggccgacgtgcgcctccgcctggccaatgtgtcggagcgggacgggggcgagtacctctgtcgagccaccaatttcataggcgtggccgagaaggccttttggctgagcgttcacgggccccgagcagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacaatgttatggaacagttcaatcctgggctgcgaaatttaataaacctggggaaaaattatgagaaagctgtaaacgctatgatcctggcaggaaaagcctactacgatggagtggccaagatcggtgagattgccactgggtcccccgtgtcaactgaactgggacatgtcctcatagagatttcaagtacccacaagaaactcaacgagagtcttgatgaaaattttaaaaaattccacaaagagattatccatgagctggagaagaagatagaacttgacgtgaaatatatgaacgcaactctaaaaagataccaaacagaacacaagaataaattagagtctttggagaaatcccaagctgagttgaagaagatcagaaggaaaagccaaggaagccgaaacgcactcaaatatgaacacaaagaaattgagtatgtggagaccgttacttctcgtcagagtgaaatccagaaattcattgcagatggttgcaaagaggctctgcttgaagagaagaggcgcttctgctttctggttgataagcactgtggctttgcaaaccacatacattattatcacttacagtctgcagaactactgaattccaagctgcctcggtggcaggagacctgtgttgatgccatcaaagtgccagagaaaatcatgaatatgatcgaagaaataaagaccccagcctctacccccgtgtctggaactcctcaggcttcacccatgatcgagagaagcaatgtggttaggaaagattacgacaccctttctaaatgctcaccaaagatgccccccgctccttcaggcagagcatataccagtcccttgatcgatatgtttaataacccagccacggctgccccgaattcacaaagggtaaataattcaacaggtacttccgaagatcccagtttacagcgatcagtttcggttgcaacgggactgaacatgatgaagaagcagaaagtgaagaccatcttcccgcacactgcgggctccaacaagaccttactcagctttgcacagggagatgtcatcacgctgctcatccccgaggagaaggatggctggctctatggagaacacgacgtgtccaaggcgaggggttggttcccgtcgtcgtacacgaagttgctggaagaaaatgagacagaagcagtgaccgtgcccacgccaagccccacaccagtgagaagcatcagcaccgtgaacttgtctgagaatagcagtgttgtcatccccccacccgactacttggaatgcttgtccatgggggcagctgccgacaggagagcagattcggccaggacgacatccacctttaaggccccagcgtccaagcccgagaccgcggctcctaacgatgccaacgggactgcaaagccgccttttctcagcggagaaaacccctttgccactgtgaaactccgcccgactgtgacgaatgatcgctcggcacccatcattcgatga

SEQ ID NO: 5 - DNA, 4989 bp, Homo sapiens
  source 1..4989: mol_type = other DNA
  variation 1..2301: note = FGFR2
atggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgagatcatggaggaaacaaatacgcagattgcttggccatcaaaactgaagatcggagccaaatccaagaaagatccccatattaaggtttctggaaagaaagaagatgttaaagaagccaaggaaatgatcatgtctgtcttagacacaaaaagcaatcgagtcacactgaagatggatgtttcacatacagaacattcacatgtaatcggcaaaggtggcaacaatattaaaaaagtgatggaagaaaccggatgccatatccactttccagattccaacaggaataaccaagcagaaaaaagcaaccaggtatctatagcgggacaaccagcaggagtagaatctgcccgagttagaattcgggagctgcttcctttggtgctgatgtttgagctaccaattgctggaattcttcaaccggttcctgatcctaattccccctctattcagcatatatcacaaacgtacaatatttcagtatcatttaaacagcgttcccgaatgtatggtgctactgtcatagtacgagggtctcagaataacactagtgctgtgaaggaaggaactgccatgctgttagaacatcttgctgggagcttagcatcagctattcctgtgagcacacaactagatattgcagctcaacatcatctctttatgatgggtcgaaatgggagcaacatcaaacatatcatgcagagaacaggtgctcagatccactttcctgatcccagtaatccacaaaagaaatctaccgtctacctccagggcaccattgagtctgtctgtcttgcaaggcaatatctcatgggttgtcttcctcttgtgttgatgtttgatatgaaggaagaaattgaagtagatccacaattcattgcgcagttgatggaacagcttgatgtcttcatcagtattaaaccaaagcccaaacagccaagcaagtctgtgattgtgaaaagtgttgagcgaaatgccttaaatatgtatgaagcaaggaaatgtctcctcggacttgaaagcagtggggttaccatagcaaccagtccatccccagcatcctgccctgccggcctggcatgtcccagcctggatatcttagcttcagcaggccttggactcactggactaggtcttttgggacccaccaccttatctctgaacacttcaacaaccccaaactcactcttgaatgctcttaatagctcagtcagtcctttgcaaagtccaagttctggtacacccagccccacattatgggcacccccacttgctaatacttcaagtgccacaggtttttctgctataccacaccttatgattccatctactgcccaagccacattaactaatattttgttgtctggagtgcccacctatgggcacacagctccatctccccctcctggcttgactcctgttgatgtccatatcaacagtatgcagaccgaaggcaaaaaaatctctgctgctttaaatggacatgcacagtctccagatataaaatatggtgcaatatccacttcatcacttggagaaaaagtgctgagtgcaaatcacggggatccgtccatccagacaagtgggtctgagcagacatctcccaaatcaagccccactgaaggttgtaatgatgcttttgttgaagtaggcatgcctcgaagtccttcccattctgggaatgctggtgacttgaaacagatgatgtgtccctccaaggtttcctgtgccaaaaggcagacagtggaactattgcaaggcacgaaaaactcacacttacacagcactgacaggttgctctcagaccctgaactgagtgctaccgaaagccctttggctgacaagaaggctccagggagtgagcgcgctgcagagagggcagcagctgcccagcaaaactccgaaagggcccaccttgctccacggtcatcatatgtcaacatgcaggcatttgactatgaacagaagaagctattagccaccaaagctatgttaaagaaaccagtggtgacggaggtcagaacgcccacaaatacctggagtggcctgggtttttctaaatccatgccagctgaaactatcaaggagttgagaagggccaatcatgtgtcctataagcccacaatgacaaccacttatgagggctcatccatgtccctttcacggtccaacagtcgtgagcacttgggaggtggaagcgaatctgataactggagagaccgaaatggaattggacctggaagtcatagtgaatttgcagcttctattggcagccctaagcgtaaacaaaacaaatcaacggaacactatctcagcagtagcaattacatggactgcatttcctcgctgacaggaagcaatggctgtaacttaaatagctctttcaaaggttctgacctccctgagctcttcagcaaactgggcctgggcaaatacacagatgttttccagcaacaagagatcgatcttcagacattcctcactctcacagatcaggatctgaaggagctgggaataactacttttggtgccaggaggaaaatgctgcttgcaatttcagaactaaataaaaaccgaagaaagctttttgaatcgccaaatgcacgcacctctttcctggaaggtggagcgagtggaaggctaccccgtcagtatcactcagacattgctagtgtcagtggccgctggtag

SEQ ID NO: 6 - DNA, 3213 bp, Homo sapiens
  source 1..3213: mol_type = other DNA
  variation 1..2301: note = FGFR2
atggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgagatggcagatgatcagggctgtattgaagagcagggggttgaggattcagcaaatgaagattcagtggatgctaagccagaccggtcctcgtttgtaccgtccctcttcagtaagaagaagaaaaatgtcaccatgcgatccatcaagaccacccgggaccgagtgcctacatatcagtacaacatgaattttgaaaagctgggcaaatgcatcataataaacaacaagaactttgataaagtgacaggtatgggcgttcgaaacggaacagacaaagatgccgaggcgctcttcaagtgcttccgaagcctgggttttgacgtgattgtctataatgactgctcttgtgccaagatgcaagatctgcttaaaaaagcttctgaagaggaccatacaaatgccgcctgcttcgcctgcatcctcttaagccatggagaagaaaatgtaatttatgggaaagatggtgtcacaccaataaaggatttgacagcccactttaggggggatagatgcaaaacccttttagagaaacccaaactcttcttcattcaggcttgccgagggaccgagcttgatgatggcatccaggccgactcggggcccatcaatgacacagatgctaatcctcgatacaagatcccagtggaagctgacttcctcttcgcctattccacggttccaggctattactcgtggaggagcccaggaagaggctcctggtttgtgcaagccctctgctccatcctggaggagcacggaaaagacctggaaatcatgcagatcctcaccagggtgaatgacagagttgccaggcactttgagtctcagtctgatgacccacacttccatgagaagaagcagatcccctgtgtggtctccatgctcaccaaggaactctacttcagtcaatag

SEQ ID NO: 7 - DNA, 268 bp, Homo sapiens
  source 1..268: mol_type = other DNA
  variation 82: note = single nucleotide polymorphism
tcggaccgcggcaactacacctgcgtcgtggagaacaagtttggcagcatccggcagacgtacacgctggacgtgctggagtgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctca

SEQ ID NO: 8 - DNA, 378 bp, Homo sapiens
  source 1..378: mol_type = other DNA
  variation 174: note = single nucleotide polymorphism
gaccgcggcaactacacctgcgtcgtggagaacaagtttggcagcatccggcagacgtacacgctggacgtgctgggtgagggccctggggcggcgcgggggtgggggcggcagtggcggtggtggtgagggagggggtggcccctgagcgtcatctgcccccacagagcgctgcccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaaggtgggccaccgtgtgcacgt

SEQ ID NO: 9 - DNA, 234 bp, Homo sapiens
  source 1..234: mol_type = other DNA
  variation 88: note = single nucleotide polymorphism
gcgggcaattctattgggttttctcatcactctgcgtggctggtggtgctgccagccgaggaggagctggtggaggctgacgaggcgtgcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccg

SEQ ID NO: 10 - DNA, 301 bp, Homo sapiens
  source 1..301: mol_type = other DNA
  variation 158: note = single nucleotide polymorphism
ctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtgtgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagc